(19) United States
(12) Patent Application Publication
(10) Pub. No.: US 2015/0057179 A1
(43) Pub. Date: Feb. 26, 2015

(54) Methods and Compositions for hte Diagnosis and Treatment of Thyroid Cancer

(71) Applicants: PAUL WALFISH, TORONTO (CA); RANJU RALHAN, THORNHILL (CA)

(72) Inventors: PAUL WALFISH, TORONTO (CA); RANJU RALHAN, THORNHILL (CA); K.W. MICHAEL SIU, TORONTO (CA)

(73) Assignees: PAUL WALFISH, TORONTO (CA); RANJU RALHAN, THORNHILL (CA)

(21) Appl. No.: 14/476,222

(22) Filed: Sep. 3, 2014

Related U.S. Application Data
(63) Continuation of application No. 13/102,638, filed on May 6, 2011, now abandoned.
(60) Provisional application No. 61/332,381, filed on May 7, 2010.

(51) Int. Cl. G01N 33/574

(52) U.S. Cl. CPC G01N 33/57407; G01N 2333/70503
USPC 506/9; 436/501; 435/6.12; 435/7.23

(57) ABSTRACT

Methods for detecting thyroid cancer or thyroid cancer status in a subject are described comprising measuring novel markers or polynucleotides encoding the markers in a sample from the subject. The invention also provides localization or imaging methods for thyroid cancer, and kits for carrying out the methods of the invention. The invention also contemplates therapeutic applications for thyroid cancer employing the novel markers, polynucleotides encoding the markers, and/or binding agents for the markers.

### CROSS REFERENCE TO RELATED APPLICATION

This application is a Continuation of U.S. application Ser. No. 13/102,638, filed on May 6, 2011, which claims the benefit under 35 USC §119(e) of U.S. Provisional Patent Application No. 61/332,381, filed May 7, 2010. Both such prior applications are incorporated herein in their entirety.

### FIELD OF THE INVENTION

The invention relates to compositions, kits, and methods for detecting, screening for, diagnosing, monitoring, and characterizing thyroid cancer.

### BACKGROUND OF THE INVENTION

Thyroid cancers are the most common malignancy of the endocrine system. [1] There is currently a lack of molecular markers to predict the aggressiveness of thyroid cancers. Currently fine-needle aspiration (FNA) is the most accurate preoperative technique for diagnosis of thyroid nodules. However, even when using ultrasound-guided FNA, inconclusive biopsy results are quite common (10-20% of all cases). [2] These patients normally undergo subsequent surgery to remove their thyroid gland—an invasive procedure that is usually unnecessary as the majority of the suspected lesions turn out to be benign (>80%). [3] Patients with inconclusive results and malignant tumors are also at risk for not undergoing adequate treatment as many of them undergo an initial thyroid lobectomy that must be followed up with another surgery to complete the thyroidectomy following diagnosis. [3] Additionally, an issue also arises due to the fact that while most papillary thyroid cancers are non-aggressive with limited to no metastasis, a small percentage are in-fact aggressive and may produce distant metastasis leading to higher mortality. [1] This establishes an urgent need for identifying biomarkers to distinguish benign thyroid nodules from malignant and aggressive carcinomas.

### SUMMARY OF THE INVENTION

The present invention relates to markers of thyroid cancer. In particular, polypeptides and domains thereof disclosed in Table 1 (collectively referred to herein as “Polypeptide Thyroid Cancer Markers”), and polynucleotides encoding such polypeptides and domains thereof (collectively referred to herein as “Polynucleotide Thyroid Cancer Markers”) constitute biomarkers for thyroid cancer. Polypeptide Thyroid Cancer Markers and Polynucleotide Thyroid Cancer Markers, and portions or fragments thereof, are sometimes collectively referred to herein as “Thyroid Cancer Markers”.

Thus, Thyroid Cancer Markers and agents that interact with the Thyroid Cancer Markers, may be used in detecting, screening for, diagnosing, characterizing, and monitoring thyroid cancer (i.e., monitoring progression of the cancer or the effectiveness of a therapeutic treatment) or for assisting same, in the identification of subjects with a predisposition to thyroid cancer, and in determining patient survival. In aspects of the invention, the Thyroid Cancer Markers are used in characterizing the aggressiveness of a thyroid cancer. In some aspects of the invention, the Thyroid Cancer Markers are used to determine metastatic potential or patient survival.

A method of the invention wherein Thyroid Cancer Marker(s) are assayed can have enhanced sensitivity and/or specificity relative to a method assaying other markers. The enhanced clinical sensitivity may be about a 5-10% increase, in particular 6-9% increase, more particularly 8% increase in sensitivity. In an embodiment, a method of the invention where one or more Thyroid Cancer Marker(s) detected in tumor samples provides a thyroid cancer clinical sensitivity of at least about 80 to 99%, in particular 90 to 95%, more particularly 91%, 92%, 93%, or 94% thyroid cancer clinical sensitivity. In embodiments of the invention the clinical sensitivity of a method of the invention can be greater than about 80 to 90%, more particularly greater than about 80 to 85%, most particularly greater than about 83%, 84%, or 85%. Clinical sensitivity and specificity may be determined using methods known to persons skilled in the art.

In accordance with methods of the invention, a Thyroid Cancer Marker in a sample can be assessed by detecting the presence in the sample of (a) a polypeptide or polypeptide fragment corresponding to the marker; (b) a transcribed nucleic acid or fragment thereof having at least a portion with which the marker is substantially identical; and/or (c) a transcribed nucleic acid or fragment thereof, wherein the nucleic acid hybridizes with the marker.

One aspect of the invention provides a method for detecting or characterizing a thyroid cancer in a patient comprising determining the status of Thyroid Cancer Markers in a sample obtained from the patient, wherein an abnormal status in the sample indicates the presence of the condition. Another aspect of the invention provides a method of screening for thyroid cancer in a patient comprising identifying a patient at risk of having thyroid cancer or in need of screening and determining the status of Thyroid Cancer Markers in a sample obtained from the patient, wherein an abnormal status of the markers indicates the presence of thyroid cancer. In some embodiments, the patient is at risk of developing a specific type of thyroid cancer and the abnormal status indicates the presence of the specific type of thyroid cancer.

Another aspect provides a diagnostic method comprising identifying a patient who is a candidate for treatment for thyroid cancer and determining the status of Thyroid Cancer Markers in a sample obtained from the patient, wherein an abnormal status of the markers in the sample indicates that treatment is desirable or necessary.

In aspects of the invention, the abnormal status can be an elevated status, low status or negative status. In an embodiment of the invention for detecting or diagnosing thyroid cancer or a type of thyroid cancer the abnormal status is an elevated status.

In an aspect of the invention, a method is provided for detecting Thyroid Cancer Markers associated with thyroid cancer in a patient comprising or consisting essentially of: (a) obtaining a sample from a patient; b) detecting or identifying in the sample one or more Thyroid Cancer Markers, in particular a Thyroid Cancer Marker set out in Table 1; and (c) comparing the detected amount with an amount detected for a standard.

In an aspect, the invention provides a method for diagnosing or screening for thyroid cancer in a subject, the method comprising: (a) contacting a sample from a subject with reagents capable of measuring levels of target Thyroid Cancer Markers, in particular Thyroid Cancer Markers set out in Table 1; and (b) providing a diagnosis of thyroid cancer in said subject based on a significant difference in the level of the Thyroid Cancer Markers in the sample from the subject over a control level obtained from similar samples taken from subjects who do not have thyroid cancer or from the subject at a different time.

In an embodiment of the invention, a method is provided for detecting one or more of the Thyroid Cancer Markers in a patient comprising or consisting essentially of: (a) obtaining a sample from a patient; (b) detecting or identifying in the sample one or more of the Thyroid Cancer Markers, in particular Thyroid Cancer Markers set out in Table 1; and (c) comparing the detected amounts with amounts detected for a standard.

In embodiments of the invention, the Thyroid Cancer Markers are chosen from tyrosine-protein kinase receptor UFO (AXL) [SEQ ID NO. 1], activated leukocyte cell adhesion molecule (ALCAM)/CD166 [SEQ ID NO. 2], and prothymosin alpha (PTMA) [SEQ ID NO. 3], and optionally galectin-3 [SEQ ID NO. 4]. In embodiments of the invention, the Thyroid Cancer Markers comprise or are selected from the group consisting of or consisting essentially of tyrosine-protein kinase receptor UFO (AXL) and activated leukocyte cell adhesion molecule (ALCAM)/CD166.

In embodiments of the invention, the Thyroid Cancer Markers comprise or are selected from the group consisting of or consisting essentially of tyrosine-protein kinase receptor UFO (AXL), activated leukocyte cell adhesion molecule (ALCAM)/CD166, and prothymosin alpha (PTMA).

In embodiments of the invention, the Thyroid Cancer Marker is biotinidase [SEQ ID NO. 5]. In embodiments of the invention, the Thyroid Cancer Markers comprises clusterin [SEQ ID NO. 6], activated leukocyte cell adhesion molecule (ALCAM)/CD166, amyloid precursor protein like protein 2 (APLP2) [SEQ ID NO.7 ], tyrosine-protein kinase receptor UFO (AXL), nucleolin [SEQ ID NO. 8], PTMA, amyloid precursor protein (APP) [SEQ ID NO. 9], 14-3-3 zeta [SEQ ID NO. 10], SET [SEQ ID NO.11], PKM2 [SEQ ID NO. 12] or hnRNPK [SEQ ID NO. 13].

In embodiments of the invention, the Thyroid Cancer Markers comprise or are selected from the group consisting of or consisting essentially of tyrosine-protein kinase receptor UFO (AXL), activated leukocyte cell adhesion molecule (ALCAM)/CD166, prothymosin-alpha (PTMA), nucleolin, biotinidase, amyloid precursor protein (APP), APLP2 and calsyntenin-1 [SEQ ID NO. 14].

In embodiments of the invention, the Thyroid Cancer Markers measured additionally comprise or are selected from the group consisting of or consisting essentially of essentially of clusterin, Dickkopf-related protein 3 (DKK-3) [SEQ ID NO. 15], nidogen-1 [SEQ ID NO. 16], gelsolin [SEQ ID NO. 17], and nucleobindin [SEQ ID NO. 18].

In embodiments of the invention, the Thyroid Cancer Markers measured additionally comprise or are selected from the group consisting essentially of CYR61 [SEQ ID NO. 19], E-cadherin [SEQ ID NO. 20] and prothymosin-alpha.

In embodiments of the invention, the Thyroid Cancer Markers measured additionally comprise or are selected from the group consisting essentially of □-Enolase [SEQ ID NO. 21], and dystroglycan 1 [SEQ ID NO. 22].

In embodiments of the invention, the Thyroid Cancer Markers measured additionally comprised or selected from the group consisting essentially of clusterin, Dickkopf-related protein 3 (DKK-3), nidogen-1, gelsolin, nucleobindin, melanoma-associated antigen [SEQ ID NO. 23], osteopontin [SEQ ID NO. 24] and plasminogen activator urokinase [SEQ ID NO. 25].

In embodiments of the invention, the Thyroid Cancer Markers measured additionally comprise or are selected from the group consisting essentially of clusterin, Dickkopf-related protein 3 (DKK-3), nidogen-1, gelsolin, nucleobindin, melanoma-associated antigen, osteopontin, plasminogen activator urokinase, nucleotin, CYR61, E-cadherin and prothymosin-alpha.

In embodiments of this aspect of the invention, the Thyroid Cancer Markers measured comprise two, three, four, five, six, seven, eight, nine, ten or more markers set out in Table 1.

The invention further provides a non-invasive non-surgical method for detection or diagnosis of thyroid cancer in a subject comprising: obtaining a sample (e.g., fluid sample) from the subject; subjecting the sample to a procedure to detect Thyroid Cancer Marker(s); detecting or diagnosing thyroid cancer by comparing the levels of Thyroid Cancer Marker(s) to the levels of Thyroid Cancer Marker(s) obtained from a control subject with no thyroid cancer or a lower grade of thyroid cancer. In embodiments of this method of the invention, the Thyroid Cancer Marker(s) are one or more of the markers set out in Table 1.

The invention contemplates a method for determining the aggressiveness or stage of thyroid cancer comprising producing a profile of levels of Thyroid Cancer Markers, and other markers associated with thyroid cancer, in cells from a patient, and comparing the profile with a reference to identify a profile for the test cells indicative of aggressiveness or stage of disease. In an aspect, the markers are Polypeptide Thyroid Cancer Markers and the profile is generated using a mass spectrometer.

In particular aspects, methods of the invention are used to diagnose the stage of thyroid cancer in a subject or characterizing thyroid cancer in a subject. In an embodiment, the method comprises comparing: (a) levels of one or more Thyroid Cancer Markers set out in Table 1, in particular the follicular thyroid cancer markers, papillary thyroid cancer markers or aggressive/metastatic thyroid cancer markers set out in Table 1, from a sample from the patient; and (b) levels of the Thyroid Cancer Markers in control samples of the same type obtained from patients without thyroid cancer or control patients with a different stage of thyroid cancer (e.g., low grade thyroid cancer) or from another sample from the subject, wherein altered levels of Thyroid Cancer Markers, relative to the corresponding levels in the control samples is an indication that the patient is afflicted with a more aggressive or metastatic thyroid cancer.

In embodiments, follicular thyroid cancer is diagnosed and the Thyroid Cancer Markers are one or more of the Thyroid Cancer Markers for follicular thyroid cancer set out in Table 1.

In aspects of the invention, a method is provided for diagnosing follicular thyroid cancer in a patient comprising or consisting essentially of: (a) detecting or identifying in the sample one, two or three of the Thyroid Cancer Markers for follicular thyroid cancer set out in Table 1, and optionally one or more additional Thyroid Cancer Marker set out in Table 1 or 2; and (b) comparing the detected amount with an amount detected for a standard, wherein a significant difference in the Thyroid Cancer Markers is indicative of follicular thyroid cancer. In embodiments of the invention relating to follicular thyroid cancer, the Thyroid Cancer Markers comprise or are selected from the group consisting essentially of calmodulin [SEQ ID NO. 26], CD44 antigen [SEQ ID NO. 27], fibronectin [SEQ ID NO. 28], ubiquitin A-52 residue ribosomal protein fusion product [SEQ ID NO. 29], and basement membrane specific heparin sulfate core protein [SEQ ID NO. 30].

In embodiments, papillary thyroid cancer is diagnosed and the Thyroid Cancer Markers are one or more of the Thyroid Cancer Markers for papillary thyroid cancer set out in Table 1. In a particular embodiment of the invention, a method is provided for diagnosing or detecting papillary thyroid cancer in a patient comprising or consisting essentially of: (a) detecting or identifying in the sample one or more Thyroid Cancer Markers for papillary thyroid cancer set out in Table 1, and optionally one or more additional Thyroid Cancer Marker set out in Table 1 and 2; and (b) comparing the detected amount with an amount detected for a standard, wherein significant difference in the amount of the Thyroid Cancer Markers is indicative of papillary thyroid cancer. In embodiments of the invention relating to papillary thyroid cancer, the Thyroid Cancer Markers comprise versican [SEQ ID NO. 31], nucleolin and/or prothymosin-alpha. In embodiments of the invention relating to papillary thyroid cancer, the Thyroid Cancer Markers comprise versican, nucleolin and/or prothymosin-alpha, and optionally CYR61 and/or E-cadherin.

In embodiments of the invention relating to papillary thyroid cancer, the Thyroid Cancer Markers comprise one or more of, or are selected from the group consisting of or consisting essentially of gamma-glutamyl hydrolase [SEQ ID NO. 32], lysyl oxidase-like 2 [SEQ ID NO. 33], biotinidase, and nidogen-1. The Thyroid Cancer Markers for detecting papillary thyroid cancer may additionally comprise cysteine-rich angiogenic inducer, 61 (CYR61) and/or E-cadherin. In embodiments of the invention relating to papillary thyroid cancer, the Thyroid Cancer Markers comprise two, three, four, five, six, seven, eight, nine, ten or all the for papillary thyroid cancer markers set out in Table 1.

In embodiments, aggressive thyroid cancer, in particular ATC, is diagnosed and the Thyroid Cancer Markers are one or more of the Thyroid Cancer Markers for aggressive/metastatic thyroid cancer set out in Table 1. In a particular aspect of the invention, a method is provided for detecting Thyroid Cancer Markers associated with aggressive or metastatic thyroid cancer, in a patient comprising or consisting essentially of: (a) obtaining a sample from a patient; (b) detecting in the sample one or more Thyroid Cancer Markers for aggressive/metastatic thyroid cancer set out in Table 1 and optionally one or more additional Thyroid Cancer Marker set out in Tables 1 and 2; and (c) comparing the detected amount with an amount detected for a standard or cut-off value. In embodiments of the invention relating to aggressive or metastatic thyroid cancer, the Thyroid Cancer Markers comprise two, three, four or all the aggressive/metastatic thyroid cancer markers set out in Table 1. In embodiments of the invention relating to aggressive or metastatic thyroid cancer, the Thyroid Cancer Markers comprise prothymosin-alpha. In embodiments of the invention relating to aggressive or metastatic thyroid cancer, the Thyroid Cancer Markers comprise one or more or all of, or are selected from the group consisting essentially of activated leukocyte cell adhesion molecule (ALCAM)/CD166, tyrosine-protein kinase receptor UFO (AXL), amyloid precursor protein like protein 2 (APLP2), cadherin-2, prothymosin-alpha, clusterin, syndecan-4 [SEQ ID NO. 34], E-cadherin, gelsolin, hnRNP A2/B1 [SEQ ID NO. 35], nucleolin, □-MCFD2 [SEQ ID NO. 36], □-NPC2 [SEQ ID NO. 37] and SET protein.

The invention provides a method of assessing whether a patient is afflicted with thyroid cancer, the method comprising comparing: (a) levels of one or more Thyroid Cancer Markers set out in Table 1 from the patient; and (b) standard levels of Thyroid Cancer Markers in samples of the same type obtained from control patients not afflicted with thyroid cancer or with a lower grade of thyroid cancer, wherein altered levels of Thyroid Cancer Markers relative to the corresponding standard levels of Thyroid Cancer Markers is an indication that the patient is afflicted with thyroid cancer. In an embodiment of a method of the invention for assessing whether a patient is afflicted with follicular thyroid cancer (FTC), levels of one or more Thyroid Cancer Markers for follicular thyroid cancer set out in Table 1, in particular basement membrane specific heparin sulfate core protein, in a sample from the patient are compared to a standard. In an embodiment of a method of the invention for assessing whether a patient is afflicted with follicular thyroid cancer (FTC), levels of one or more Thyroid Cancer Markers for follicular thyroid cancer set out in Table 1 and optionally one or more additional Thyroid Cancer Marker set out in Tables 1 and 2, in particular calmodulin, CD44 antigen, fibronectin, ubiquitin A-52 residue ribosomal protein fusion product, and basement membrane specific heparin sulfate core protein, in a sample from the patient are compared to a standard.

In an embodiment of a method of the invention for assessing whether a patient is afflicted with follicular thyroid cancer higher levels of one or more Thyroid Cancer Markers for follicular thyroid cancer set out in Table 1 and optionally one or more additional Thyroid Cancer Marker set out in Tables 1 and 2, in particular calmodulin, CD44 antigen, fibronectin, ubiquitin A-52 residue ribosomal protein fusion product, or basement membrane specific heparin sulfate core protein, in a sample relative to a standard or corresponding normal levels, is an indication that the patient is afflicted with follicular thyroid cancer.

In an embodiment of a method of the invention for assessing whether a patient is afflicted with papillary thyroid cancers (PTC), levels of one or more Thyroid Cancer Markers for papillary thyroid cancer set out in Table 1 in a sample from the patient are compared to a standard. In an embodiment of a method of the invention for assessing whether a patient is afflicted with papillary thyroid cancers (PTC), levels of nucleolin and/or prothymosin-alpha, and optionally CYR61 and/or E-cadherin, in a sample from the patient are compared to a standard.

In an aspect of a method of the invention for assessing whether a patient is afflicted with papillary thyroid cancer higher levels of one or more Thyroid Cancer Markers for papillary thyroid cancer set out in Table 1 in a sample relative to a standard or corresponding normal levels, is an indication that the patient is afflicted with follicular thyroid cancer. In an aspect of a method of the invention for assessing whether a patient is afflicted with papillary thyroid cancer higher levels of one or both of nucleolin and/or prothymosin-alpha, and optionally CYR61 and/or E-cadherin, in a sample relative to a standard or corresponding normal levels, is an indication that the patient is afflicted with follicular thyroid cancer.

In an embodiment of a method of the invention for assessing whether a patient is afflicted with aggressive or metastatic thyroid cancer, levels of one or more Thyroid Cancer Markers for aggressive or metastatic thyroid cancer set out in Table 1 in a sample from the patient are compared to a standard. In an aspect of a method of the invention for assessing whether a patient is afflicted with aggressive or metastatic thyroid cancer higher levels of one or more Thyroid Cancer Markers aggressive or metastatic thyroid cancer set out in Table 1 in a sample relative to a standard or corresponding normal levels or levels from a patient with a lower grade of thyroid cancer, is an indication that the patient is afflicted with aggressive or metastatic thyroid cancer.

In an embodiment of a method of the invention for assessing whether a patient is afflicted with anaplastic thyroid cancer, levels of one or more Thyroid Cancer Markers for aggressive or metastatic thyroid cancer set out in Table 1 and particularly marked *** in a sample from the patient are compared to a standard, and significantly different levels of the Thyroid Cancer Markers compared to a standard are indicative of anaplastic thyroid cancer.

In an aspect of a method of the invention for assessing whether a patient is afflicted with aggressive or metastatic thyroid cancer or anaplastic thyroid cancer levels of prothymosin-alpha in a sample from the patient are compared to a standard.

In aspects of the invention, aggressive thyroid cancer, in particular ATC, is detected, diagnosed or characterized by determination of increased levels of one or more Thyroid Cancer Marker(s) aggressive or metastatic thyroid cancer set out in Table 1 and Marked *** when compared to such levels obtained from a control.

In an aspect, the invention provides a method for monitoring the progression of thyroid cancer in a patient the method comprising: (a) detecting one or more Thyroid Cancer Marker(s) set out in Table 1 and optionally Table 2 in a patient sample (e.g. biopsy sample) at a first time point; (b) repeating step (a) at a subsequent point in time; and (c) comparing the levels detected in (a) and (b), and thereby monitoring the progression of thyroid cancer in the patient.

The invention provides a method for classifying a patient having thyroid cancer, the method comprising measuring one or more Thyroid Cancer Marker(s) set out in Table 1 and optionally Table 2 in a fluid sample, in particular serum sample, from the patient and correlating the values measured to values measured for the Thyroid Cancer Markers from thyroid cancer patients stratified in classification groups. The method can be used to predict patient survival, wherein the Thyroid Cancer Marker(s) are predictive of survival and wherein the classification groups comprise groups of known overall survival. In aspects of this method of the invention, the Thyroid Cancer Marker(s) are selected from the follicular thyroid cancer markers, papillary thyroid cancer markers or aggressive/metastatic thyroid cancer markers in Table 1. In various embodiments the values measured can be normalized to provide more accurate quantification and to correct for experimental variations.

In aspects of the invention, Polynucleotide Thyroid Cancer Markers are detected and levels of Polynucleotide Thyroid Cancer Markers in a sample from a patient are compared with Polynucleotide Thyroid Cancer Marker levels from samples of patients without thyroid cancer, with a lower grade of thyroid cancer, or from levels from samples of the same patient. A method of the invention may employ one or more polynucleotides, oligonucleotides, or nucleic acids capable of hybridizing to Polynucleotide Thyroid Cancer Markers. The present invention relates to a method for diagnosing and characterizing thyroid cancer, more particularly the stage of thyroid cancer, in a sample from a subject comprising isolating nucleic acids, preferably mRNA, from the sample, and detecting Polynucleotide Thyroid Cancer Markers in the sample.

The invention also provides methods for determining the presence or absence of thyroid cancer or the aggressiveness or metastatic potential of a thyroid cancer in a subject in the subject comprising detecting in the sample a level of nucleic acids that hybridize to one or more Polynucleotide Thyroid Cancer Marker(s) encoding polypeptides set out in Table 1, and optionally Table 2, and comparing the level(s) with a predetermined standard or cut-off value, and therefrom determining the presence or absence of thyroid cancer or the aggressiveness or metastatic potential of a thyroid cancer in the subject in the subject. In an embodiment a method is provided for determining the aggressiveness or metastatic potential of thyroid cancer in a subject comprising (a) contacting a sample taken from the subject with oligonucleotides that hybridize to one or more polynucleotides encoding the Thyroid Cancer Markers for follicular thyroid cancer markers, papillary thyroid cancer markers and/or aggressive/metastatic thyroid cancer markers set out in Table 1; and (b) detecting in the sample a level of nucleic acids that hybridize to the oligonucleotides relative to a predetermined standard or cut-off value, and therefrom determining the aggressiveness or metastatic potential of the cancer in the subject.

In an aspect, the invention provides a method of assessing the aggressiveness or metastatic potential of a thyroid cancer in a patient, the method comprising comparing: (a) levels of one or more Polynucleotide Thyroid Cancer Marker(s) set out in Table 1, in particular follicular thyroid cancer markers, papillary thyroid cancer markers or aggressive/metastatic thyroid cancer markers set out in Table 1, in a sample from the patient; and (b) control levels of the Polynucleotide Thyroid Cancer Marker(s) in samples of the same type obtained from control patients not afflicted with thyroid cancer or a lower grade of thyroid cancer, wherein altered levels of Polynucleotide Thyroid Cancer Marker(s) relative to the corresponding control levels of the Polynucleotide Thyroid Cancer Marker(s) is an indication of the aggressiveness or metastatic potential of the thyroid cancer.

In a particular method of the invention for assessing whether a patient is afflicted with an aggressive or metastatic thyroid cancer higher levels of prothymosin-alpha or nucleolin, in a sample relative to the corresponding control levels is an indication that the patient is afflicted with an aggressive or metastatic thyroid cancer.

Within certain embodiments, the amount of nucleic acid that is mRNA is detected via amplification reactions such as polymerase chain reaction (PCR) using, for example, at least one oligonucleotide primer that hybridizes to a Polynucleotide Thyroid Cancer Marker(s) or a complement of such polynucleotide. Within other embodiments, the amount of mRNA is detected using a hybridization technique, employing an oligonucleotide probe that hybridizes to a Polynucleotide Thyroid Cancer Marker(s), or a complement thereof.

When using mRNA detection, the method may be carried out by combining isolated mRNA with reagents to convert to cDNA according to standard methods; treating the converted cDNA with amplification reaction reagents along with an appropriate mixture of primers to produce amplification products; and analyzing the amplification products to detect the presence of Polynucleotide Thyroid Cancer Marker(s) in the sample. For mRNA the analyzing step may be accomplished using RT-PCR analysis to detect the presence of Polynucleotide Thyroid Cancer Marker(s). The analysis step may be accomplished by quantitatively detecting the presence of Polynucleotide Thyroid Cancer Marker(s) in the amplification product, and comparing the quantity of Polynucleotide Thyroid Cancer Marker(s), detected against a panel of expected values for known presence or absence in normal and malignant samples derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a Polynucleotide Thyroid Cancer Marker(s) to produce amplification products; (d) analyzing the amplification products to detect an amount of mRNA Polynucleotide Thyroid Cancer Marker(s); and (e) comparing the amount of mRNA to an amount detected against a panel of expected values for normal and malignant samples (e.g., samples from patients with a different stage of thyroid cancer) derived using similar nucleic acid primers.

In particular embodiments of the invention, the methods described herein utilize the Polynucleotide Thyroid Cancer Markers placed on a microarray so that the expression status of each of the markers is assessed simultaneously. In a particular aspect, the invention provides a microarray comprising a defined set of genes whose expression is significantly altered by a thyroid cancer or procedure. The invention further relates to the use of the microarray as a prognostic tool to predict thyroid cancer or status of a thyroid cancer. In an embodiment, the invention provides for oligonucleotide arrays comprising marker sets described herein. The microarrays provided by the present invention may comprise probes to markers able to distinguish thyroid cancer. In particular, the invention provides oligonucleotide arrays comprising probes to a subset or subsets of at least 5 or 10 gene markers up to a full set of markers which distinguish thyroid cancer.

Protein based methods can also be used for determining the presence or absence of thyroid cancer or the aggressiveness or metastatic potential of a thyroid cancer in a sample from a subject. Thyroid Cancer Markers may be detected using a binding agent for Thyroid Cancer Markers, preferably antibodies specifically reactive with Thyroid Cancer Markers, or parts thereof.

The invention provides a method of assessing whether a patient is afflicted with thyroid cancer which comprises comparing: (a) levels of one or more Polypeptide Thyroid Cancer Markers set out in Table 1, and optionally Table 2 in a sample from the patient; and (b) control levels of the Polypeptide Thyroid Cancer Markers in a non-cancer sample or sample from a patient with a lower grade of thyroid cancer or from a sample from the patient taken at another time, wherein significantly different levels of Polypeptide Thyroid Cancer Markers in the sample from the patient compared with the control levels is an indication that the patient is afflicted with thyroid cancer.

In another aspect the invention provides methods for determining the presence or absence of thyroid cancer or the aggressiveness or metastatic potential of a thyroid cancer or classifying thyroid cancer in a patient comprising the steps of (a) contacting a biological sample obtained from a patient with a binding agent that specifically binds to one or more Polypeptide Thyroid Cancer Marker(s) set out in Table 1 and optionally Table 2; and (b) detecting in the sample an amount of the Polypeptide Thyroid Cancer Marker(s) that binds to the binding agent(s), relative to a predetermined standard or cut-off value, and therefrom determining the presence or absence of thyroid cancer, the aggressiveness or metastatic potential of thyroid cancer or the stage of thyroid cancer in the patient.

In an embodiment, the invention relates to a method for detecting, diagnosing, staging and monitoring thyroid cancer in a subject by quantitating one or more Polypeptide Thyroid Cancer Marker(s) in a biological sample from the subject comprising (a) reacting the biological sample with an antibody specific for one or more Polypeptide Thyroid Cancer Marker(s) set out in Table 1, and optionally Table 2, which is directly or indirectly labeled with a detectable substance; and (b) detecting the detectable substance.

In another embodiment the invention provides a method of using antibodies to detect expression of Polypeptide Thyroid Cancer Marker(s) in a sample, the method comprising: (a) combining antibodies specific for Polypeptide Thyroid Cancer Marker(s) with a sample under conditions which allow the formation of antibody:protein complexes; and (b) detecting complex formation, wherein complex formation indicates expression of Polypeptide Thyroid Cancer Marker(s)in the sample. Expression may be compared with standards and is diagnostic of thyroid cancer, stage of thyroid cancer, or the aggressiveness or metastatic potential of the thyroid cancer.

Polypeptide Thyroid Cancer Markers can be determined by constructing an antibody microarray in which binding sites comprise immobilized, preferably monoclonal, antibodies specific to a substantial fraction of marker-derived thyroid cancer proteins of interest.

In an aspect, the invention provides a method for monitoring the progression of thyroid cancer in a patient, the method comprising: (a) detecting one or more Polypeptide Thyroid Cancer Marker(s) set out in Table 1, and optionally Table 2, in a patient sample at a first time point; and (b) repeating step (a) at a subsequent point in time; and (c) comparing the levels detected in (a) and (b), and thereby monitoring the progression of thyroid cancer in the patient.

The invention further relates to a method of assessing the efficacy of a therapy for thyroid cancer in a patient. This method comprises comparing: (a) levels of one or more Thyroid Cancer Markers set out in Table 1, and optionally Table 2, in a first sample obtained from the patient prior to providing at least a portion of the therapy to the patient; and (b) levels of the Thyroid Cancer Markers in a second sample obtained from the patient following therapy. Significantly different levels of Thyroid Cancer Markers in the second sample, relative to the first sample, can be an indication that the therapy is efficacious for inhibiting thyroid cancer. In an embodiment, the method is used to assess the efficacy of a therapy for inhibiting thyroid cancer and significantly different levels of one or more Thyroid Cancer Markers for follicular thyroid cancer, papillary thyroid cancer and/or aggressive/metastatic thyroid cancer in Table 1, in the second sample relative to the first sample, is an indication that the therapy is efficacious for inhibiting the cancer or metastasis. The therapy may be any therapy for treating thyroid cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden, aggressiveness or metastatic potential of the tumor.

The invention provides marker sets for diagnosing or characterizing thyroid cancer and uses thereof. A marker set may comprise a plurality of polypeptides and/or polynucleotides encoding such polypeptides comprising or consisting of at least 2, 3, 4, 5, 6, 7, 8, 9, 10, 11, 12, 13, 14, or 15 of the markers of Table 1. In specific aspects, the markers consist of at least 2, 3, 4, 5, 6, 7, 8, 9, or 10 polypeptides of Table 1. In an aspect the protein marker sets comprise or consist of protein clusters, or proteins in pathways comprising markers of Table 1. In embodiments of the invention, a marker is provided which is selected from the group consisting of the polypeptides set forth in Table 1 which polypeptides are up-regulated biomarkers in thyroid cancer, in particular follicular thyroid cancer, papillary cancer or metastatic/aggressive thyroid cancer.

The invention also provides a diagnostic composition comprising Thyroid Cancer Markers or agents that interact with Thyroid Cancer Markers. In particular, the invention provides a diagnostic composition comprising Polypeptide Thyroid Cancer Markers, or agents that bind to such markers, or hybridize to or amplify Polynucleotide Thyroid Cancer Markers. In an embodiment, the composition comprises a probe that specifically hybridizes to a Polynucleotide Thyroid Cancer Marker or a fragment thereof, and a probe that specifically hybridizes to a Polynucleotide Thyroid Cancer Marker or a fragment thereof. In another embodiment a composition is provided comprising a specific primer(s) pair capable of amplifying a Polynucleotide Thyroid Cancer Marker using polymerase chain reaction methodologies. In a still further embodiment, the composition comprises a binding agent(s) (e.g. antibody) that binds to a Polypeptide Thyroid Cancer Marker or a fragment thereof. Probes, primers, and binding agents can be labeled with a detectable substance. In an embodiment, a diagnostic composition of the invention comprises one or more antibodies specific for a Thyroid Cancer Marker in Table 1. In an embodiment, a diagnostic composition of the invention comprises one or more primers that amplify polynucleotides encoding a Thyroid Cancer Marker in Table 1.

In another aspect, the invention relates to use of an agent that interacts with a Thyroid Cancer Marker in the manufacture of a composition for diagnosing thyroid cancer, in particular the aggressiveness or metastatic potential of a thyroid cancer.

The methods of the invention may also comprise detecting additional markers associated with thyroid cancer, for example the markers listed in Table 2, more particularly galectin-3, thyroglobulin, and E-cadherin. Further, the amount of Thyroid Cancer Markers may be mathematically combined with other markers of thyroid cancer. In an embodiment the invention provides a method for detecting or diagnosing thyroid cancer in a subject comprising: (a) determining the amount of one or more Thyroid Cancer Markers in Table 1 in a sample from the subject; (b) determining the amount of other markers associated with thyroid cancer in particular markers comprising or selected from the markers listed in Table 2 or from the group consisting of or consisting essentially of galectin-3, thyroglobulin and E-cadherin, in the sample; (c) mathematically combining the results of step (a) and step (b) to provide a mathematical combination; and (d) comparing or correlating the mathematical combination to the presence of thyroid cancer, stage of thyroid cancer or aggressiveness or metastatic potential of thyroid cancer.

In a particular embodiment the invention provides a method for diagnosing the aggressiveness of thyroid cancer in a subject comprising: (a) determining the amount of one or more Thyroid Cancer Marker(s) for aggressive or metastatic thyroid cancer set out in Table 1 from the subject; (b) determining the amount of Thyroid Cancer Marker(s) in the sample; (c) determining the amount of one or more of E-cadherin, CYR61, melanoma-associated antigen, osteopontin, and plasminogen activator urokinase in the sample; (d) mathematically combining the results of step (a) and step (b), and optionally step (c) to provide a mathematical combination; and (e) comparing or correlating the mathematical combination to the aggressiveness of the thyroid cancer. The combination is preferably compared to a mathematical combination for a predetermined standard. In particular aspects, the invention provides a method for detecting, characterizing or diagnosing thyroid cancer by determining the combination of Thyroid Cancer Markers and one or more of the markers listed in Table 2, or one or both of galectin-3 and thyroglobulin in a sample from a subject.

The invention also includes kits for carrying out methods of the invention. In an aspect the invention provides a kit for detecting, diagnosing, screening for, monitoring, predicting or characterizing thyroid cancer comprising Thyroid Cancer Markers. In a particular aspect, the invention provides a test kit for diagnosing screening for, monitoring, predicting or characterizing thyroid cancer in a subject which comprises an agent that interacts with a Thyroid Cancer Marker(s). In an embodiment, the kit is for assessing whether a patient is afflicted with follicular thyroid cancer and it comprises reagents for identifying and/or assessing levels of the Thyroid Cancer Markers for follicular thyroid cancer in Table 1. In an embodiment, the kit is for assessing whether a patient is afflicted with papillary thyroid cancer and it comprises reagents for identifying and/or assessing levels of the Thyroid Cancer Markers for papillary thyroid cancer in Table 1. In an embodiment, the kit is for assessing whether a patient is afflicted with aggressive or metastatic thyroid cancer and it comprises reagents for identifying and/or assessing levels of the Thyroid Cancer Markers for aggressive or metastatic thyroid cancer in Table 1.

The invention contemplates an in vivo method comprising administering to a mammal one or more agent that carries a label for imaging and binds to a Thyroid Cancer Marker, and then imaging the mammal. According to a preferred aspect of the invention, an in vivo method for imaging thyroid cancer is provided comprising: (a) injecting a patient with an agent that binds to a Thyroid Cancer Marker(s), the agent carrying a label for imaging the thyroid cancer; (b) allowing the agent to incubate in vivo and bind to the Thyroid Cancer Marker(s); and (c) detecting the presence of the label localized to the thyroid cancer. In an embodiment of the invention the agent is an antibody which recognizes the Thyroid Cancer Marker(s). In another embodiment of the invention the agent is a chemical entity which recognizes the Thyroid Cancer Marker(s). The agent carries a label to image the Thyroid Cancer Marker(s). Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The invention also contemplates the localization or imaging methods described herein using multiple markers for thyroid cancer.

In an aspect, the invention provides antagonists (e.g. antibodies) specific for a Thyroid Cancer Marker set out in Table 1 that can be used therapeutically to destroy or inhibit the growth of thyroid cancer cells.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows the verification of secreted proteins in thyroid cancer patients' sera by immunoblotting in tissue culture media and sera of thyroid cancer patients. Protein samples were prepared from cultured cells (50-75 μg), their respective serum-free media (SFM; 5 μg), and the sera (50 μg) of five thyroid cancer patients (2 non-aggressive PTC, FTC, aggressive PTC (metastatic thyroid cancer) and ATC and one normal sample (‘normal’, S-2145, Sigma). As protein loading controls, lysates were examined for actin expression, while SFM and patients' sera were stained with Ponceau S (Sigma). Western blot analysis confirmed the detection of five proteins, namely clusterin, CYR 61, α-enolase, nucleolin and prothymosin-alpha in the thyroid carcinoma patients' sera and in the whole-cell lysate and conditioned media (CM) of the thyroid carcinoma cell lines as shown in panel A. The panel B shows increase in α-MCFD2 [SEQ ID NO. 36], α-NPC2 [SEQ ID NO. 37], E cadherin and calsyntenin1, in normal and thyroid cancer patients' sera. The panels C and D show decrease in α-Enolase probed with rabbit polyclonal antibody (H300; Santa Cruz Biotech) and a mouse monoclonal antibody (ab54979; Abcam). Levels of the 48-kDa α-Enolase protein expressed relative to the level of this protein in benign thyroid nodule taken as 1.0 were observed to decrease in ATC and metastatic PTC suggesting a reduction that correlated with disease aggressiveness. FIG. 1 panels E, F and G show immunohistochemical analysis of α-Enolase expression in non-aggressive PTC (E), metastatic PTC (F), and normal thyroid tissue (G) (Original magnification ×400). Reduced cytoplasmic and nuclear staining was observed in PTC and metastatic PTC as compared to the normal thyroid tissues; reduction was more pronounced in the aggressive PTC tissue.

FIG. 2, panels B and C show increased nuclear and cytoplasmic expression of prothymosin alpha (PTMA) by immunohistochemical analysis in human thyroid carcinoma tissues—(B) PTC and (C) ATC as compared to the normal tissue (A) (Original magnification ×400). Panel D shows increased nuclear nucleolin expression in normal thyroid tissue, while the PTC (E) and ATC (F) showed reduced nuclear and increased cytoplasmic expression of nucleolin (Original magnification ×400). Panels G and H depict scatter plot analysis of immunohistochemical scoring of PTMA and nucleolin in different subtypes of thyroid carcinomas. Tissues were scored based on percentage positivity and immunostaining intensity. Sections were scored as positive if epithelial cells showed immunoreactivity in the plasma membrane, cytoplasm, and/or nucleus when observed by evaluators blinded to the clinical history and outcome. The percentage positive scores were assigned according to the following scale: 0, <10% cells; 1, 10-30% cells; 2, 30-50% cells; 3, 50-70% cells; and 4, >70%. The intensity of the staining was also scored semi-quantitatively as follows: 0, no staining; 1, mild; 2, moderate; and 3, intense. The total score (0-7) was obtained by adding the percentage positivity scores and intensity scores for each section. (G) ATC displayed elevated nuclear expression of PTMA compared to normal thyroid adjacent to benign thyroid disease, insular and papillary thyroid carcinomas. ATC cases displayed strikingly elevated cytoplasmic expression of PTMA compared to PTC and insular cells. PTC staining of cytoplasmic PTMA was low. Insular (poorly differentiated) thyroid carcinomas also demonstrated an increased expression of cytoplasmic PTMA compared to PTC and normal thyroid adjacent to benign thyroid disease. (H) Nuclear expression of nucleolin was seen in all thyroid carcinoma subtypes and normal thyroid adjacent to benign thyroid disease examined. Faint cytoplasmic expression was also observed in ATC cases only. These findings suggest increase in nuclear and cytoplasmic expression of PTMA with disease aggressiveness, while increased cytoplasmic nucleolin was observed in ATC.

FIG. 3, panel A, B and C show detection and verification of serum biotinidase in patients with thyroid cancer. Serum proteins from patients with benign (n=8), non-aggressive PTC (n=6) and metastatic PTC (n=6) were immunodepleted, resolved on SDS-PAGE and probed with α-biotinidase rabbit polyclonal antibody K-17 (Santa Cruz Biotech; 1:1000 dilution) or mouse monoclonal (ab54886; Abcam) antibody. The 75 kDa biotinidase protein band levels were quantified using ImageQuant software (GE Biosciences) and are expressed as a proportion of the highest benign band intensity per blot. Biotinidase levels decreased from benign levels for nearly all of the PTC cases that correlated with disease aggressiveness. Panels D-G show immunohistochemical analysis of biotinidase expression in thyroid tissues. Stronger cytoplasmic staining was observed in the non-aggressive (E) and metastatic PTC (F), whereas nuclear levels of biotinidase decreased with increasing thyroid cancer aggressiveness. Original magnification ×400. Scatter plot analysis of biotinidase cytoplasmic and nuclear expression is shown in panels H and I, benign (n=3), non-aggressive (n=21) and aggressive (n=6) PTC cases with respective mean and standard error bars indicated. Stronger cytoplasmic staining above the benign levels was observed for all non-aggressive and aggressive thyroid cancer cases. In the nucleus, biotinidase expression was found to decrease with increased tumor aggressiveness.

FIG. 4, panels A and B show verification of serum clusterin levels in thyroid cancer patients. Serum proteins from patients with benign (n=8), non-aggressive (n=6) and aggressive (n=6) PTC were immunodepleted treatment prior to electrophoresis. To detect clusterin protein, blots were prepared and probed with α-clusterin mouse monoclonal antibody (78E; Santa Cruz Biotech) at a 1:2000 dilution. The expected 40-kDa protein was detected by 78E and found to decrease in abundance from benign levels in both non-aggressive and metastatic PTC cases. This reduction was also more pronounced in patients with more aggressive disease. Panel B shows verification of serum clusterin with mouse and rabbit α-clusterin antibodies. Immunoblots containing serum proteins from benign, non-aggressive and aggressive PTC cases were probed with α-clusterin 78E mouse monoclonal or rabbit polyclonal (ab92548; Abcam) antibody. Both antibodies recognized the expected 40-kDa clusterin protein and showed reduction in serum clusterin in thyroid cancer cases, particularly for aggressive PTC. Band intensities are presented as a proportion of the benign level for the respective blot. Panels C-F show verification of clusterin expression in thyroid tissues by immunohistochemical analysis. Sections from non-aggressive PTC (C), metastatic PTC (D), and normal (E) cases were probed with α-clusterin mouse monoclonal antibody. Increased cytoplasmic staining was observed from normal levels for both thyroid cancers. Furthermore, this increase was more prominent in the aggressive PTC tissue. In all cases, no detectable membrane or nuclear expression of clusterin was observed. Minimal non-specific staining was observed when sections were probed with the 2° antibody alone (F). Original magnification ×400. Panels G and H show secreted serum clusterin levels in thyroid cancer patients using ELISA. Serum samples from 39 cases (10 benign, 10 non-aggressive PTC, 19 aggressive PTC) were examined using the Human Clusterin ELISA kit (BioVendor), as recommended. Bar (G) and scatter (H) plots were generated, depicting mean and standard error values. t-test analyses indicated that a significant difference was observed in serum clusterin levels in benign and aggressive thyroid cancers (p=0.011).

FIG. 5, panels A-D show (ALCAM)/CD166 expression in human thyroid cancer tissue using immunohistochemical analysis. (A) Moderate membrane staining is observed in benign tissue. (B) Intense membrane and cytoplasmic staining in shown for stage I PTC, compared to moderate cytoplasmic staining in stage IV PTC (D). Very weak cytoplasmic staining is shown in ATC (C). Original magnification ×400. Panel E shows scatter plot analysis of (ALCAM)/CD166 cytoplasmic and membrane expression. Tissue sections from benign (n=9), non-aggressive (n=16), aggressive (n=19) PTC and ATC (n=5) cases were probed with (ALCAM)/CD166 antibody. The distribution of all immunostaining scores for cytoplasmic and membrane (ALCAM)/CD166 expression is shown, with respective mean and standard error bars indicated. Decreased membrane staining was observed for all non-aggressive and aggressive PTC and ATC cases as compared to the benign cases. Panel F shows secreted (ALCAM)/CD166 in thyroid cancer patients' sera using ELISA. Pre- and post-surgery blood samples were assessed. Non-Agg refers to non-aggressive stage I or II PTC cases with no extrathyroidal spread. Agg PTC refers to aggressive PTC cases that have lymph node metastasis, extrathyroidal invasion, and multifocality. Very aggressive refers to the more aggressive PTC cases, which are poorly differentiated and/or have distant metastasis. Pre and post-surgery non-aggressive PTC and the very aggressive post-surgery PTC (ALCAM)/CD166 levels were lower than all the other groups. The non-aggressive PTC (ALCAM)/CD166 levels were lower as compared to the other groups.

FIG. 6 shows verification of secreted proteins in thyroid cancer patients' sera. Blots containing immunodepleted serum samples (50 μg/lane) were probed with respective antibodies. AXL receptor tyrosine kinase, pyruvate kinase 3 isoform 1 variant (PKM2), APLP2 and amyloid precursor protein (APP) levels were increased in thyroid cancer patients' sera and correlated with aggressiveness. 14-3-3 zeta and protein phosphatase 2A inhibitor, SET, were detected in the sera of thyroid cancer patients as well.

FIG. 7, panels A-F show APLP2 expression in human thyroid cancer tissues using immunohistochemical analysis. (A) Benign tissues showed moderate nuclear and mild cytoplasmic staining. (B) Stage II PTC showed moderate nuclear and cytoplasmic staining. (C) Stage IV tall cell variant PTC showed stronger cytoplasmic staining compared to the other thyroid cancer subtypes, with some mild membrane staining. (D) Stage IV PTC revealed mild membrane and cytoplasmic staining compared to moderate cytoplasmic staining in multifocal stage IV PTC (E). (F) ATC tissue showed moderate cytoplasmic APLP2 staining. Original magnification ×400. Panel G shows scatter plot analysis of APLP2 nuclear and cytoplasmic expression. Tissue sections from benign (n=3), PTC (n=7) and ATC (n=1) cases were probed with APLP2 antibody. The distribution of all immunostaining scores for nuclear and cytoplasmic APLP2 expression is shown, with respective mean and standard error bars indicated.

FIG. 8 shows secreted AXL protein in thyroid cancer patient sera using an enzyme-linked immunosorbent assay. Pre- and post-surgery blood samples were assessed. Non-Agg refers to non-aggressive stage I or II PTC cases with no extrathyroidal spread. Agg PTC refers to aggressive PTC cases that have lymph node metastasis, extrathyroidal invasion, and multifocality. Very-Agg refers to the more aggressive PTC cases, which are poorly differentiated and/or have distant metastasis. Lt Post-Non-Agg refers to long-term post-surgery non-aggressive PTC blood samples taken 4-30 years after surgery with patients having no residual cancer. In long-term post-surgery non-aggressive PTC and post-surgery very aggressive PTC, AXL sera levels were lower compared to their respective pre-surgery levels.

FIG. 9 show immunohistochemical analysis of APP, 14-3-3 zeta, AXL, SET, PKM2 and hnRNPK in benign tissues and PTC. Cytoplasmic expressions of APP and 14-3-3 zeta proteins were observed in PTCs, in contrast to the absent cytoplasmic and nuclear staining in benign thyroid tissues. Minimal nuclear expression and no cytoplasmic expression of AXL were observed in benign thyroid tissues. Cytoplasmic accumulation of AXL protein was found in the PTC cases examined. Strong nuclear expression and no cytoplasmic expression were observed for SET in benign thyroid tissues. In PTC, SET was found to show reduced nuclear expression. PKM2 displayed nuclear and cytoplasmic expression in benign thyroid tissues. In PTC, cytoplasmic accumulation of the PKM2 protein was found to increase, often with a loss of nuclear expression. hnRNPK was found in the nucleus of benign thyroid tissues. This nuclear expression was found to decrease, with a concomitant increase in cytoplasmic hnRNPK in the PTC cases observed. Original magnification ×400.

FIG. 10 shows scatter plot analysis of APP, SET, AXL, 14-3-3 zeta, PKM2 and hnRNPK proteins expression. Tissue sections from benign (n=13) and papillary thyroid carcinomas (PTC, n=13) cases were probed with respective antibodies. The distribution of all immunostaining scores for these proteins expression is shown, with respective mean and standard error bars indicated. Increased cytoplasmic staining of APP (A) and 14-3-3 zeta (D) was observed in PTC cases as compared to benign. Nuclear staining of SET protein was observed in PTC and benign cases (B). Minimal nuclear expression and no cytoplasmic expression of AXL were observed in benign thyroid tissues (C). Cytoplasmic accumulation of AXL protein was found in the PTC cases examined (C). PKM2 displayed nuclear and cytoplasmic expression in benign thyroid tissues (E). In PTC, cytoplasmic accumulation of PKM2 protein was found to increase, often with a loss of nuclear expression (E). hnRNPK was found in the nucleus of benign thyroid tissues (F). This nuclear expression was found to decrease, with a concomitant increase in cytoplasmic hnRNPK in the PTC cases observed (F).

FIG. 11 shows immunohistochemical analysis of APP, AXL, PKM2 and SET proteins in mouse xenografts of human PTC-derived (BCPAP) and ATC-derived (C643) thyroid carcinoma cell lines. In agreement with the observations in human PTC, APP was observed to stain in the cytoplasm of PTC xenografts. Increased cytoplasmic expression of APP was found in the ATC xenografts. Weak cytoplasmic expression of AXL was observed in ATC xenografts. In anaplastic mouse xenografts, decreased nuclear expression of PKM2 in a majority of cells was observed, along with cytoplasmic immunostaining compared to papillary mouse xenografts. Consistent with the observations in human tissues, strong SET nuclear expression was observed in papillary mouse xenografts. Decreased nuclear staining of SET was observed in ATC xenografts, along with cytoplasmic expression of the protein. Original magnification ×400.

### DETAILED DESCRIPTION OF THE INVENTION

The invention relates to newly discovered correlations between expression of Thyroid Cancer Markers and thyroid cancer, in particular stage, aggressiveness or metastatic potential of a thyroid cancer. The Thyroid Cancer Markers described herein provide methods for diagnosing, detecting, predicting, monitoring or characterizing thyroid cancer, in particular stage, aggressiveness or metastatic potential of a thyroid cancer. Methods are provided for screening for, diagnosing or detecting the presence or absence of thyroid cancer, papillary, follicular or aggressive or metastatic thyroid cancer in a sample, and for monitoring the progression of thyroid cancer, as well as providing information about characteristics of a thyroid carcinoma that are relevant to the diagnosis and characterization of thyroid carcinoma in a patient.

### Glossary

Unless defined otherwise, all technical and scientific terms used herein have the same meaning as commonly understood by one of ordinary skill in the art to which this invention belongs. The following definitions supplement those in the art and are directed to the present application and are not to be imputed to any related or unrelated case. Although any methods and materials similar or equivalent to those described herein can be used in the practice of the invention, particular materials and methods are described herein.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. As used herein and in the appended claims, the singular forms “a”, “an”, and “the” include plural reference unless the context clearly dictates otherwise.

The term “thyroid cancer” refers to any malignant process of the thyroid gland. Examples of thyroid cancers include, but are not limited to, papillary thyroid carcinoma, follicular variant of papillary thyroid carcinoma, follicular carcinoma, Hurthle cell tumor, anaplastic thyroid carcinoma, medullary thyroid cancer, thyroid lymphoma, poorly differentiated thyroid cancer and thyroid angiosarcoma. In aspects of the invention, the thyroid cancer is papillary thyroid carcinoma. In aspects of the invention, the thyroid cancer is a follicular variant of papillary thyroid carcinoma or follicular carcinoma (‘also referred to herein as “follicular thyroid cancer”). In aspects of the invention, the thyroid cancer is medullary thyroid cancer. In aspects of the invention, the thyroid cancer is an aggressive cancer or has metastatic potential, in particular an aggressive medullary or follicular thyroid cancer or a medullary or follicular thyroid cancer with metastatic potential. In aspects of the invention, the thyroid cancer is anaplastic thyroid carcinoma (ATC).

“Metastatic potential” refers to the ability or possibility of a cancer cell moving from the initial site (i.e. thyroid) to other sites in the body.

The term “detect” or “detecting” includes assaying, or otherwise screening or establishing the presence or absence of the target marker(s), subunits, or combinations of reagent bound targets, and the like, or assaying for ascertaining, establishing, classifying monitoring, predicting or otherwise determining one or more factual characteristics of a thyroid cancer such as aggressiveness, metastatic potential or patient survival, or assisting in same. A standard may correspond to levels quantitated for samples from control subjects with no disease or early stage disease (e.g., low grade thyroid cancer such as papillary thyroid cancer) or from other samples of the subject.

The term “sample” and the like mean a material known or suspected of expressing or containing Thyroid Cancer Markers, or binding agents such as antibodies specific for Polypeptide Thyroid Cancer Markers. The sample may be derived from a biological source (“biological sample”), such as tissues, extracts, or cell cultures, including cells (e.g. tumor cells), cell lysates, and biological or physiological fluids, such as, for example, whole blood, plasma, serum, saliva, cerebral spinal fluid, sweat, urine, milk, peritoneal fluid and the like. A sample may be used directly as obtained from the source or following a pretreatment to modify the character of the sample, such as preparing plasma from blood, diluting viscous fluids, and the like. In certain aspects of the invention, the sample is a fluid sample. In certain aspects of the invention the sample is serum, plasma, whole blood, urine or saliva. In certain particular aspects of the invention the sample is serum. In certain aspects of the invention, the sample is a human physiological fluid, such as human serum. In aspects of the invention, the sample comprises cells (or nuclei obtained from the cells) from different sites of a tumor.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). As will be appreciated by those skilled in the art, the target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA). (i.e., cRNA; see, for example., Linsley & Schelter, U.S. patent application Ser. No. 09/411,074, or U.S. Pat. Nos. 5,545,522, 5,891,636 or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Molecular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+ RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemiluminescent label, radiolabel, and colorimetric label.

Target polynucleotides from a patient sample can be labeled differentially from polynucleotides of a standard. The standard can comprise target polynucleotides from normal individuals (e.g. those not afflicted with or pre-disposed to thyroid cancer, in particular pooled from samples from normal individuals or patients with a different disease stage). The target polynucleotides can be derived from the same individual, but taken at different time points, and thus indicate the efficacy of a treatment by a change in expression of the markers, or lack thereof, during and after the course of treatment.

The terms “subject”, “patient” and “individual” are used interchangeably herein and refer to a warm-blooded animal such as a mammal that is afflicted with thyroid cancer, or suspected of having thyroid cancer, being pre-disposed to thyroid cancer, being screened for thyroid cancer or at risk for thyroid cancer. The term includes but is not limited to domestic animals, sports animals, primates and humans. Preferably, the terms refer to a human.

As used herein, the term subject “suspected of having thyroid cancer” refers to a subject that presents one or more symptoms indicative of a thyroid cancer (e.g., a noticeable lump or mass) or is being screened for a cancer (e.g., during a routine physical). A subject suspected of having thyroid carcinoma may also have one or more risk factors. A subject suspected of having thyroid cancer has generally not been tested for cancer. However, a “subject suspected of having thyroid cancer” encompasses an individual who has received an initial diagnosis but for whom the stage of cancer is not known. The term further includes people who once had cancer (e.g., an individual in remission). As used herein, the term subject “at risk for thyroid cancer” refers to a subject with one or more risk factors for developing thyroid carcinoma. Risk factors include, but are not limited to, gender, age, genetic predisposition, environmental exposure, previous incidents of cancer, preexisting non-cancer diseases, and lifestyle. As used herein, the term “characterizing thyroid cancer in a subject” refers to the identification of one or more properties of a cancer sample in a subject, including but not limited to the subject's prognosis or survival. Cancers may be characterized by the identification of the expression of one or more markers, including but not limited to, the Thyroid Cancer Markers disclosed herein.

“Thyroid Cancer Markers for papillary thyroid cancer set out in Table 1” include the markers identified by “*” in Table 1, namely markers chosen from the markers numbered 4, 12, 25 and 26, and polynucleotides encoding same.

“Thyroid Cancer Markers for follicular thyroid cancer set out in Table 1” include the markers identified by “**” in Table 1, namely markers chosen from the markers numbered 21, 22 and 28, and polynucleotides encoding same.

“Thyroid Cancer Markers for aggressive/metastatic thyroid cancer set out in Table 1” include the markers identified by “***” in Table 1, namely markers chosen from the markers numbered 15, 17, 29, 30, 31, 32, 33, 35, 36, 38, 39, 40, 42, 43, 45, 46, 47, 49-58, 60, 65-75, 77, 78, 80, 81 and 83, 88 and polynucleotides encoding same.

Thyroid Cancer Markers include polypeptide or protein markers including without limitation a native-sequence polypeptide, a polypeptide variant, a chimeric protein or fusion protein, isoforms, complexes, all homologs, fragments, precursors, and modified forms and derivatives of the markers (i.e., Polypeptide Thyroid Cancer Markers).

“Polypeptide” and “protein” are used interchangeably herein and indicate at least one molecular chain of amino acids linked through covalent and/or non-covalent bonds. The terms include peptides, oligopeptides, and proteins, and post-translational modifications of the polypeptides, e.g. glycosylations, acetylations, phosphorylations, and the like. Protein fragments, analogues, mutated or variant proteins, fusion proteins, and the like, are also included within the meaning of the terms.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 10%, 20%, 30%, 40%, 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95%, 97%, or 99% amino acid sequence identity with a native-sequence polypeptide. Particular polypeptide variants have at least 70-80%, 85%, 90%, 95%, 97% or 99% amino acid sequence identity to the sequences identified in Table 1 or 2. Such variants include, for instance, polypeptides wherein one or more amino acid residues are added to, or deleted from, the N- or C-terminus of the full-length or mature sequences of the polypeptide, including variants from other species, but exclude a native-sequence polypeptide. In aspects of the invention variants retain the immunogenic activity of the corresponding native-sequence polypeptide.

Sequence identity of two amino acid sequences or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Polypeptide variants include polypeptides comprising amino acid sequences sufficiently identical to or derived from the amino acid sequence of a native polypeptide which includes fewer amino acids than the full-length polypeptides. A portion or fragment of a polypeptide can be a polypeptide which is for example, 3-5, 8-10, 10, 15, 15-20, 20, 25, 30, 35, 40, 45, 50,60, 70, 80, 90, 100 or more amino acids in length. Portions or fragments in which regions of a polypeptide are deleted can be prepared by recombinant techniques and can be evaluated for one or more functional activities such as the ability to form antibodies specific for a polypeptide. A portion or fragment of a polypeptide may comprise a domain of the polypeptide, in particular an extracellular domain or intracellular domain.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain, several of which are known in the art.

A naturally occurring allelic variant may contain conservative amino acid substitutions from the native polypeptide sequence or it may contain a substitution of an amino acid from a corresponding position in polypeptide homolog, for example, a murine polypeptide.

A polypeptide disclosed herein includes chimeric or fusion proteins. A “chimeric protein” or “fusion protein” comprises all or part (preferably biologically active) of the polypeptide operably linked to a heterologous polypeptide (i.e., a different polypeptide). Within the fusion protein, the term “operably linked” is intended to indicate that the polypeptide and the heterologous polypeptide are fused in-frame to each other. The heterologous polypeptide can be fused to the N-terminus or C-terminus of the polypeptide. A useful fusion protein is a GST fusion protein in which a polypeptide is fused to the C-terminus of GST sequences. Another example of a fusion protein is an immunoglobulin fusion protein in which all or part of a polypeptide is fused to sequences derived from a member of the immunoglobulin protein family. Chimeric and fusion proteins can be produced by standard recombinant DNA techniques.

Polypeptides used in the methods disclosed herein may be isolated from a variety of sources, such as from human tissue types or from other sources, or prepared by recombinant or synthetic methods, or by any combination of these and similar techniques.

“Polynucleotide” refers to a polymeric form of nucleotides of any length, either ribonucleotides or deoxyribonucleotides. The term includes double- and single-stranded DNA and RNA, modifications such as methylation or capping and unmodified forms of the polynucleotide. The terms “polynucleotide” and “oligonucleotide” are used interchangeably herein. A polynucleotide may, but need not, include additional coding or non-coding sequences, or it may, but need not, be linked to other molecules and/or carrier or support materials. Polynucleotide Thyroid Cancer Markers for use in the methods of the invention may be of any length suitable for a particular method. In certain applications the term refers to antisense nucleic acid molecules (e.g. an mRNA or DNA strand in the reverse orientation to a sense Polynucleotide Thyroid Cancer Markers).

Polynucleotide Thyroid Cancer Markers include polynucleotides encoding Polypeptide Thyroid Cancer Markers, including a native-sequence polypeptide, a polypeptide variant including a portion of a Polypeptide Thyroid Cancer Marker, an isoform, precursor, a chimeric protein, complexes, homologs, fragments, precursors, and modified forms and derivatives of the markers.

Polynucleotides used in the methods of the invention include complementary nucleic acid sequences, and nucleic acids that are substantially identical to these sequences (e.g. at least about 10%, 20%, 30%, 40%, or 45%, preferably 50%, 55%, 60%, 65%, 70%, 75%, 80%, 85%, 90%, 95%, 97%, 98%, or 99% sequence identity).

Polynucleotides also include sequences that differ from a nucleic acid sequence due to degeneracy in the genetic code. As one example, DNA sequence polymorphisms within the nucleotide sequence of a Thyroid Cancer Marker disclosed herein may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides which may be used in the methods disclosed herein include nucleic acids that hybridize under stringent conditions, preferably high stringency conditions to a nucleic acid sequence of a Polynucleotide Thyroid Cancer Marker. Appropriate stringency conditions which promote DNA hybridization are known to those skilled in the art, or can be found in Ausubel et al., (eds) Current Protocols in Molecular Biology, John Wiley & Sons, N.Y. (1989), 6.3.1-6.3.6. Generally, stringent conditions may be selected that are about 5° C. lower than the thermal melting point (Tm) for the specific sequence at a defined ionic strength and pH. The Tm is the temperature (under defined ionic strength, pH, and nucleic acid concentration) at which 50% of the probes complementary to a target sequence hybridize at equilibrium to the target sequence. Generally, stringent conditions will be those in which the salt concentration is less than about 1.0M sodium ion or other salts (e.g. about 0.01 to 1.0M sodium ion) and the temperature is at least about 30° C. for short probes, primers or oligonucleotides (e.g. 10-50 nucleotides) and at least 60° C. for longer probes, primers and oligonucleotides. For example, a hybridization may be conducted at 6.0×sodium chloride/sodium citrate (SSC) at about 45° C., followed by a wash of 2.0×SSC at 50° C., or at 42° C. in a solution containing 6×SCC, 0.5% SDS and 50% formamide followed by washing in a solution of 0.1×SCC and 0.5% SDS at 68° C.

Polynucleotide Thyroid Cancer Markers also include truncated nucleic acids or nucleic acid fragments and variant forms of the nucleic acids disclosed or referenced herein that arise by alternative splicing of an mRNA corresponding to a DNA. A fragment of a polynucleotide includes a polynucleotide sequence that comprises a contiguous sequence of approximately at least about 6 nucleotides, in particular at least about 8 nucleotides, more particularly at least about 10-12 nucleotides, and even more particularly 15-20 nucleotides that correspond to (i.e. identical or complementary to), a region of the specified nucleotide sequence.

“Significantly different” levels of markers or a “significant difference” in marker levels in a patient sample compared to a control or standard (e.g. normal levels, levels from a different disease stage, or levels in other samples from a patient) may represent levels that are higher or lower than the standard error of the detection assay, preferably the levels are at least about 1.5, 2, 3, 4, 5, 6, 7, 8, 9 or 10 times higher or lower, respectively, than the control or standard.

“Microarray” and “array,” refer to nucleic acid or nucleotide arrays or protein or peptide arrays that can be used to detect biomolecules associated with thyroid cancer, for instance to measure gene expression. A variety of arrays are available commercially, such as, for example, as the in situ synthesized oligonucleotide array GeneChip™ made by Affymetrix, Inc. or the spotted cDNA array, LifeArray™ made by Incyte Genomics Inc.

“Binding agent” refers to a substance such as a polypeptide, antibody, ribosome, or aptamer that specifically binds to a Polypeptide Thyroid Cancer Marker. A binding agent, in particular an antibody, that “specifically binds” or “binds” (used interchangeably herein) to a target or an antigen or epitope is a term well understood in the art, and methods to determine specific binding are also well known in the art. A binding agent “specifically binds” to a target if it binds with greater affinity, avidity, more readily, and/or with greater duration than it binds to other substances. It will be appreciated that an antibody that specifically binds to a first target may or may not specifically or preferentially bind to a second target. Thus, “specific binding” does not necessarily require (although it can include) exclusive binding but generally refers to preferential binding. Binding properties may be assessed using an ELISA, which may be readily performed by those skilled in the art (see for example, Newton et al, Develop. Dynamics 197: 1-13, 1993). In an embodiment of the invention, antibodies are reactive against a polypeptide marker if they bind with a Kₐ of greater than or equal to 10⁻⁷ M.

A binding agent may be a ribosome, with or without a peptide component, a RNA or DNA molecule, or a polypeptide. A binding agent may be a polypeptide that comprises a Polypeptide Thyroid Cancer Marker sequence, a peptide variant thereof, or a non-peptide mimetic of such a sequence. By way of example a Polypeptide Thyroid Cancer Marker sequence may be a peptide portion of the polypeptide that is capable of modulating a function mediated by the polypeptide.

An aptamer includes a DNA or RNA molecule that binds to nucleic acids and proteins. An aptamer that binds to a Thyroid Cancer Marker can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to synthetic antibodies, monoclonal antibodies, polyclonal antibodies, recombinant antibodies, antibody fragments (such as Fab, Fab′, F(ab′)2), dAb (domain antibody; see Ward, et al, 1989, Nature, 341:544-546), antibody heavy chains, intrabodies, humanized antibodies, human antibodies, antibody light chains, single chain Fᵥₛ (scFv) (e.g., including monospecific, bispecific etc), anti-idiotypic (ant-Id) antibodies, proteins comprising an antibody portion, chimeric antibodies (for example, antibodies which contain the binding specificity of murine antibodies, but in which the remaining portions are of human origin), derivatives, such as enzyme conjugates or labeled derivatives, diabodies, linear antibodies, disulfide-linked Fvs (sdFv), multispecific antibodies (e.g., bispecific antibodies), epitope-binding fragments of any of the above, and any other modified configuration of an immunoglobulin molecule that comprises an antigen recognition site of the required specificity. An antibody includes an antibody of any type (e.g. IgA, IgD, IgE, IgG, IgM and IgY), any class (e.g. IgG1, IgG2, IgG3, IgG4, IgA1 and IgA2), or any subclass (e.g. IgG2a and IgG2b), and the antibody need not be of any particular type, class or subclass. In certain embodiments of the invention the antibodies are IgG antibodies or a class or subclass thereof. An antibody may be from any animal origin including birds and mammals (e.g. human, murine, donkey, sheep, rabbit, goat, guinea pig, camel, horse, or chicken).

A “recombinant antibody” includes antibodies that are prepared, expressed, created or isolated by recombinant means, such as antibodies expressed using a recombinant expression vector transfected into a host cell, antibodies isolated from recombinant, combinatorial antibody libraries, antibodies isolated from an animal (e.g. a mouse or cow) that is transgenic and/or transchromosomal for human immunoglobin genes, or antibodies prepared, expressed, created or isolated by any other means that involves slicing of immunoglobulin gene sequences to other DNA sequences.

A “monoclonal antibody” refers to an antibody obtained from a population of homogenous or substantially homogenous antibodies. Generally each monoclonal antibody recognizes a single epitope on an antigen. In aspects of the invention, a monoclonal antibody is an antibody produced by a single hybridoma or other cell, and it specifically binds to only a Thyroid Cancer Marker as determined, for example by ELISA or other antigen-binding or competitive binding assay known in the art. The term is not limited to a particular method for making the antibody and for example they may be produced by the hybridoma method or isolated from phage libraries using methods known in the art.

Antibodies, including monoclonal and polyclonal antibodies, fragments and chimeras, may be prepared using methods well known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Aced Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for polypeptide markers may also be obtained from scientific or commercial sources.

The “status” of a marker refers to the presence, absence or extent/level of the marker or some physical, chemical or genetic characteristic of the marker. Such characteristics include without limitation, expression level, activity level, structure (sequence information), copy number, post-translational modification etc. The status of a marker may be directly or indirectly determined. In some embodiments status is determined by determining the level of a marker in the sample. The “level” of an element in a sample has its conventional meaning in the art, and includes quantitative determinations (e.g. mg/mL, fold change, etc) and qualitative determinations (e.g. determining the presence or absence of a marker or determining whether the level of the marker is high, low or even present relative to a standard).

The term “abnormal status” means that a marker's status in a sample is different from a reference status for the marker. A reference status may be the status of the marker in samples from normal subjects, averaged samples from subjects with the condition or sample(s) from the same subject taken at different times. An abnormal status includes an elevated, decreased, present or absent marker(s). Determining the level of a marker in a sample may include determining the level of the marker in a sample and abnormal status could be either lower levels (including undetectable levels) or higher levels (including any amount over zero) compared to a standard. A subject may have an increased likelihood of a condition disclosed herein if the status of a marker in the subject's sample is correlated with the condition (e.g. a level of the marker is closer to a standard or reference or is present in levels that exceed some threshold value where exceeding that value is correlated with the condition). A subject with an increased likelihood of a condition disclosed herein includes a subject with an abnormal status for a marker and as such the subject has a higher likelihood of the condition than if the subject did not have that status.

An “elevated status” means one or more characteristics of a marker are higher than a standard. In aspects of the invention, the term refers to an increase in a characteristic as compared to a standard. A “low status” means one or more characteristics of a marker are lower than a standard. In aspects of the invention, the term refers to a decrease in a characteristic as compared to a standard. A “negative status” means that one or more characteristic of a marker is absent or undetectable.

### General Methods

A variety of methods can be employed for the diagnostic and prognostic evaluation of thyroid cancer involving Thyroid Cancer Markers and the identification of subjects with a predisposition to such disorders. Such methods may, for example, utilize Polynucleotide Thyroid Cancer Markers and fragments thereof, and binding agents (e.g. antibodies) directed against Polypeptide Thyroid Cancer Markers including peptide fragments. In particular, the polynucleotides and antibodies may be used, for example, for (1) the detection of the presence of polynucleotide mutations, or the detection of either over- or under-expression of mRNA, relative to a non-disorder state or the qualitative or quantitative detection of alternatively spliced forms of polynucleotide transcripts which may correlate with certain conditions or susceptibility toward such conditions; and (2) the detection of either an over- or an under-abundance of polypeptides relative to a non-disorder state or the presence of a modified (e.g., less than full length) polypeptide which correlates with a disorder state, or a progression toward a disorder state.

The methods described herein may be used to evaluate the probability of the presence of malignant cells, for example, in a group of cells freshly removed from a host. Such methods can be used to detect tumors, quantitate and monitor their growth, and help in the diagnosis and prognosis of disease. For example, significantly different levels of one or more markers in Table 1 are indicative of thyroid cancer.

The methods of the invention require that the amount of Thyroid Cancer Markers quantitated in a sample from a subject being tested be compared to a predetermined standard or cut-off value. A standard may correspond to levels quantitated for another sample or an earlier sample from the subject, or levels quantitated for a control sample, in particular a sample from a subject with a lower grade cancer. Levels for control samples from healthy subjects or cancer subjects may be established by prospective and/or retrospective statistical studies. Healthy subjects who have no clinically evident disease or abnormalities may be selected for statistical studies. Diagnosis may be made by a finding of statistically different levels of Thyroid Cancer Markers compared to a control sample or previous levels quantitated for the same subject.

The invention also contemplates the methods described herein using multiple markers for thyroid cancer. Therefore, the invention contemplates a method for analyzing a biological sample for the presence of Thyroid Cancer Markers and other markers that are specific indicators of thyroid cancer. The methods described herein may be modified by including reagents to detect the markers or polynucleotides encoding the markers. Examples of other markers include without limitation the markers listed in Table 2 or markers comprising or selected from the group comprising galectin-3, thyroglobulin, E-cadherin, and galectin-3, in particular galectin-3.

### Nucleic Acid Methods

As noted herein thyroid cancer, in particular the stage or aggressiveness of a thyroid cancer, may be detected based on the level of Polynucleotide Thyroid Cancer Markers in a sample. Techniques for detecting nucleic acid molecules such as polymerase chain reaction (PCR) and hybridization assays are well known in the art.

Probes may be used in hybridization techniques to detect polynucleotides. The technique generally involves contacting and incubating nucleic acids obtained from a sample from a patient or other cellular source with a probe under conditions favorable for the specific annealing of the probes to complementary sequences in the nucleic acids (e.g. under stringent conditions as discussed herein). After incubation, the non-annealed nucleic acids are removed, and the presence of nucleic acids that have hybridized to the probe if any are detected. Nucleotide probes for use in the detection of polynucleotide sequences in samples may be constructed using conventional methods known in the art. The probes may comprise DNA or DNA mimics corresponding to a portion of an organism's genome, or complementary RNA or RNA mimics. The nucleic acids can be modified at the base moiety, at the sugar moiety, or at the phosphate backbone. DNA can be obtained using standard methods such as polymerase chain reaction (PCR) amplification of genomic DNA or cloned sequences. Computer programs known in the art can be used to design primers with the required specificity and optimal amplification properties.

A nucleotide probe may be labeled with a detectable substance such as a radioactive label which provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleic acids to be detected and the amount of nucleic acids available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect Polynucleotide Thyroid Cancer Markers, preferably in human cells. The nucleotide probes may also be useful in the diagnosis of thyroid cancer, involving Polynucleotide Thyroid Cancer Markers in monitoring the progression of thyroid cancer, or monitoring a therapeutic treatment.

The detection of polynucleotides in a sample may involve the amplification of specific gene sequences using an amplification method such as PCR, followed by the analysis of the amplified molecules using techniques known to those skilled in the art. By way of example, oligonucleotide primers may be employed in a PCR based assay to amplify a portion of a polynucleotide and to amplify a portion of a polynucleotide derived from a sample, wherein the oligonucleotide primers are specific for (i.e. hybridize to) the polynucleotides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of a Polynucleotide Thyroid Cancer Marker; that is, they are at least 10 nucleotides, and preferably at least 20 nucleotides in length. In an embodiment the primers and probes are at least about 10-40 nucleotides in length.

Hybridization and amplification reactions may also be conducted under stringent conditions as discussed herein. Hybridization and amplification techniques described herein may be used to assay qualitative and quantitative aspects of polynucleotide expression. For example, RNA may be isolated from a cell type or tissue known to express Polynucleotide Thyroid Cancer Markers, and tested utilizing the hybridization (e.g. standard Northern analyses) or PCR techniques.

In an aspect of the invention, a method is provided employing reverse transcriptase-polymerase chain reaction (RT-PCR), in which PCR is applied in combination with reverse transcription. Generally, RNA is extracted from a sample using standard techniques and is reverse transcribed to produce cDNA. The cDNA is used as a template for a polymerase chain reaction. The cDNA is hybridized to primer sets which are specifically designed against a Polynucleotide Thyroid Cancer Marker. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected thyroid cancer, an individual who is not afflicted with thyroid cancer or has early stage disease or has aggressive or metastatic disease. The reaction may be performed on several dilutions of cDNA spanning at least two orders of magnitude. A statistically significant difference in expression in several dilutions of the subject sample as compared to the same dilutions of the non-cancerous sample or early-stage cancer sample may be considered positive for the presence of cancer.

Oligonucleotides or longer fragments derived from Polynucleotide Thyroid Cancer Markers may be used as targets in a microarray. The microarray can be used to monitor the expression levels of the polynucleotides and to identify genetic variants, mutations, and polymorphisms. The information from the microarray may be used to determine gene function, to understand the genetic basis of a disorder, to diagnose a disorder, and to develop and monitor the activities of therapeutic agents. Thus, the invention also includes an array comprising Polynucleotide Thyroid Cancer Markers, and optionally other thyroid cancer markers. The array can be used to assay expression of Polynucleotide Thyroid Cancer Markers in the array. The invention allows the quantitation of expression of the polynucleotides.

The invention provides microarrays comprising Polynucleotide Thyroid Cancer Markers. In one embodiment, the invention provides a microarray for distinguishing samples associated with thyroid cancer, in particular aggressive thyroid cancer or thyroid cancer with metastatic potential comprising a positionally-addressable array of polynucleotide probes bound to a support, the polynucleotide probes comprising sequences complementary and hybridizable to Polynucleotide Thyroid Cancer Markers. In an embodiment, the array can be used to monitor the time course of expression of Polynucleotide Thyroid Cancer Markers in the array. This can occur in various biological contexts such as tumor progression. An array can also be useful for ascertaining differential expression patterns of Polynucleotide Thyroid Cancer Markers, and optionally other thyroid cancer markers in normal and abnormal cells. This may provide a battery of nucleic acids that could serve as molecular targets for diagnosis or therapeutic intervention. The preparation, use, and analysis of microarrays are well known to those skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662).

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, NY, 1988). In general, a method of the invention may comprise (a) contacting a sample from the subject with a binding agent; (b) detecting in the sample a level of polypeptide that binds to the binding agent; and (c) comparing the level of polypeptide with a predetermined standard or cut-off value. In particular aspects of the invention, the binding agent is an antibody.

In an aspect, the invention provides a diagnostic method for monitoring or diagnosing thyroid cancer in a subject by quantitating Polypeptide Thyroid Cancer Markers in a biological sample from the subject comprising reacting the sample with antibodies specific for Polypeptide Thyroid Cancer Markers which are directly or indirectly labeled with detectable substances and detecting the detectable substances.

In an aspect of the invention, a method for detecting or diagnosing thyroid cancer is provided comprising or consisting essentially of: (a) obtaining a sample suspected of containing Polypeptide Thyroid Cancer Markers; (b) contacting said sample with antibodies that specifically bind Polypeptide Thyroid Cancer Markers under conditions effective to bind the antibodies and form complexes; (c) measuring the amount of Polypeptide Thyroid Cancer Markers present in the sample by quantitating the amount of the complexes; and (d) comparing the amount of Polypeptide Thyroid Cancer Markers present in the samples with the amount of Polypeptide Thyroid Cancer Markers in a control, wherein a change or significant difference in the amount of Polypeptide Thyroid Cancer Markers in the sample compared with the amount in the control is indicative of thyroid cancer, stage of thyroid cancer, progression, aggressiveness and/or metastatic potential of the thyroid cancer.

In an embodiment, the invention contemplates a method for monitoring the progression of thyroid cancer in an individual, comprising: (a) contacting antibodies which bind to Polypeptide Thyroid Cancer Markers with a sample from the individual so as to form complexes comprising the antibodies and Polypeptide Thyroid Cancer Markers in the sample; (b) determining or detecting the presence or amount of complex formation in the sample; (c) repeating steps (a) and (b) at a point later in time; and (d) comparing the result of step (b) with the result of step (c), wherein a difference in the amount of complex formation is indicative of disease, disease stage, progression, aggressiveness and/or metastatic potential of the cancer in said individual. The amount of complexes may also be compared to a value representative of the amount of the complexes from an individual not at risk of, or afflicted with thyroid cancer at a different stage.

Antibodies specifically reactive with Polypeptide Thyroid Cancer Markers or derivatives, such as enzyme conjugates or labeled derivatives, may be used to detect Polypeptide Thyroid Cancer Markers in various samples (e.g. biological materials, in particular tissue samples). They may be used as diagnostic or prognostic reagents and they may be used to detect abnormalities in the level of Polypeptide Thyroid Cancer Markers or abnormalities in the structure, and/or temporal, tissue, cellular, or subcellular location of Polypeptide Thyroid Cancer Markers. Antibodies may also be used to screen potentially therapeutic compounds in vitro to determine their effects on thyroid cancer involving Polypeptide Thyroid Cancer Markers. In vitro immunoassays may also be used to assess or monitor the efficacy of particular therapies.

Antibodies may be used in any immunoassay that relies on the binding interaction between antigenic determinants of Polypeptide Thyroid Cancer Markers and the antibodies. Immunoassay procedures for in vitro detection of antigens in samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of Polypeptide Thyroid Cancer Markers in a sample may be accomplished by competitive or non-competitive immunoassay procedures in either a direct or indirect format. Detection of Polypeptide Thyroid Cancer Markers using antibodies can, for example involve immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. Alternatively, the binding of antibodies to Polypeptide Thyroid Cancer Markers can be detected directly using, for example, a surface plasmon resonance (SPR) procedure such as, for example, Biacore®, microcalorimetry or nano-cantilivers. These terms are well understood by those skilled in the art, and they will know, or can readily discern, other immunoassay formats without undue experimentation.

Antibodies specific for Polypeptide Thyroid Cancer Markers may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels, (e.g., FITC, rhodamine, lanthanide phosphors), luminescent labels such as luminol; and enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labelled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA. A bioluminescent compound may also be used as a detectable substance. Examples of bioluminescent detectable substances are luciferin, luciferase and aequorin.

Indirect methods may also be employed in which the primary antigen-antibody reaction is amplified by the introduction of a second antibody, having specificity for the antibody reactive against Polypeptide Thyroid Cancer Markers. By way of example, if the antibody having specificity against Polypeptide Thyroid Cancer Markers is a rabbit IgG antibody, the second antibody may be goat anti-rabbit IgG, Fc fragment specific antibody labeled with a detectable substance as described herein.

Methods for conjugating or labelling the antibodies discussed above may be readily accomplished by one of ordinary skill in the art.

Cytochemical techniques known in the art for localizing antigens using light and electron microscopy may be used to detect Polypeptide Thyroid Cancer Markers. Generally, an antibody may be labeled with a detectable substance and a Polypeptide Thyroid Cancer Marker may be localized in tissues and cells based upon the presence of the detectable substance.

In the context of the methods of the invention, the sample, binding agents (e.g. antibodies), or Polypeptide Thyroid Cancer Markers may be immobilized on a carrier or support, such as, for example, agarose, cellulose, nitrocellulose, dextran, Sephadex, Sepharose, liposomes, carboxymethyl cellulose, polyacrylamides, polystyrene, filter paper, ion-exchange resin, plastic film, nylon or silk. The support material may have any possible configuration including spherical cylindrical or flat. Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for Polypeptide Thyroid Cancer Markers may be immobilized using sheep anti-mouse IgG Fc fragment specific antibody coated on the carrier or support.

Where a radioactive label is used as a detectable substance, a Polypeptide Thyroid Cancer Marker may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T K and Diamandis E P Anal. Chem., 1992: 64:342-346 may be used with a conventional time-resolved fluorometer.

According to an embodiment of the invention, an immunoassay for detecting Polypeptide Thyroid Cancer Markers in a biological sample comprises contacting an amount of a binding agent that specifically binds to Polypeptide Thyroid Cancer Markers in the sample under conditions that allow the formation of a complex(es) comprising the binding agent and Polypeptide Thyroid Cancer Markers and determining the presence or amount of the complex(es) as a measure of the amount of the Polypeptide Thyroid Cancer Markers contained in the sample.

In accordance with an embodiment of the invention, a method is provided wherein Polypeptide Thyroid Cancer Markers antibodies are directly or indirectly labelled with enzymes, substrates for the enzymes are added wherein the substrates are selected so that the substrates, or a reaction product of an enzyme and substrate, form fluorescent complexes with lanthanide metals, preferably europium and terbium. A lanthanide metal(s) is added and Polypeptide Thyroid Cancer Markers are quantitated in the sample by measuring fluorescence of the fluorescent complexes. Enzymes are selected based on the ability of a substrate of the enzyme, or a reaction product of the enzyme and substrate, to complex with lanthanide metals. Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer.

Antibodies specific for Polypeptide Thyroid Cancer Markers may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In embodiments, antibodies specific for Polypeptide Thyroid Cancer Markers are labelled with enzymes.

Aspects of the methods of the invention involve (a) reacting a biological sample from a subject with antibodies specific for Polypeptide Thyroid Cancer Markers wherein the antibodies are directly or indirectly labelled with enzymes; (b) adding substrates for the enzymes wherein the substrates are selected so that the substrates, or reaction products of the enzymes and substrates form fluorescent complexes; (c) quantitating Polypeptide Thyroid Cancer Markers in the sample by measuring fluorescence of the fluorescent complexes; and (d) comparing the quantitated levels to levels obtained for other samples from the subject patient, or control subjects. In an embodiment, the Polypeptide Thyroid Cancer Markers are set out in Table 1 and the quantitated levels are compared to levels quantitated for normal subjects or subjects with an early stage of disease wherein a significant difference in the levels of the markers compared with the control subjects is indicative of thyroid cancer, or stage or aggressiveness of thyroid cancer.

A particular embodiment of the invention comprises the following steps: (a) incubating a biological sample with a first antibody specific for Polypeptide Thyroid Cancer Markers which is directly or indirectly labeled with a detectable substance, and a second antibody specific for Polypeptide Thyroid Cancer Markers which is immobilized; (b) separating the first antibody from the second antibody to provide a first antibody phase and a second antibody phase; (c) detecting the detectable substance in the first or second antibody phase thereby quantitating Polypeptide Thyroid Cancer Markers in the biological sample; and (d) comparing the quantitated Polypeptide Thyroid Cancer Markers with levels for a predetermined standard. The standard may correspond to levels quantitated for samples from control subjects with no disease or early stage disease or from other samples of the subject including earlier samples of the subject.

In accordance with an embodiment, the present invention provides means for determining Polypeptide Thyroid Cancer Markers in a sample by measuring Polypeptide Thyroid Cancer Markers by immunoassay. It will be evident to a skilled artisan that a variety of competitive or non-competitive immunoassay methods can be used to measure Polypeptide Thyroid Cancer Markers in serum. Competitive methods typically employ immobilized or immobilizable antibodies to Polypeptide Thyroid Cancer Markers and labeled forms of Polypeptide Thyroid Cancer Markers. Sample Polypeptide Thyroid Cancer Markers and labeled Polypeptide Thyroid Cancer Markers compete for binding to antibodies specific for Polypeptide Thyroid Cancer Markers. After separation of the resulting labeled Polypeptide Thyroid Cancer Markers that have become bound to antibody (bound fraction) from that which has remained unbound (unbound fraction), the amount of the label in either bound or unbound fraction is measured and may be correlated with the amount of Polypeptide Thyroid Cancer Markers in the test sample in any conventional manner, e.g., by comparison to a standard curve.

In another aspect, a non-competitive method is used for the determination of Polypeptide Thyroid Cancer Markers with the most common method being the “sandwich” method. In this assay, two antibodies specific for a Polypeptide Thyroid Cancer Marker are employed. One of the antibodies is directly or indirectly labeled (the “detection antibody”), and the other is immobilized or immobilizable (the “capture antibody”). The capture and detection antibodies can be contacted simultaneously or sequentially with the test sample. Sequential methods can be accomplished by incubating the capture antibody with the sample, and adding the detection antibody at a predetermined time thereafter or the detection antibody can be incubated with the sample first and then the capture antibody added. After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises Polypeptide Thyroid Cancer Marker “sandwiched” between the capture and detection antibodies. In another embodiment, the label may be measured without separating the capture antibody and liquid test mixture.

In particular sandwich immunoassays of the invention mouse polyclonal/monoclonal antibodies specific for Polypeptide Thyroid Cancer Markers and rabbit polyclonal/monoclonal antibodies specific for Polypeptide Thyroid Cancer Markers are utilized.

In a typical two-site immunometric assay for Polypeptide Thyroid Cancer Markers one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In an aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

The invention also contemplates diagnostic methods employing mass spectrometry. In an aspect, the invention relates to a method for diagnosing or screening for thyroid cancer in a subject comprising: (a) extracting proteins from a sample from the subject and producing a profile of the proteins by subjecting the proteins to mass spectrometry; and (b) comparing the profile with a profile for a reference comprising Thyroid Cancer Marker sets of the invention.

Proteins may be extracted from the samples in a manner known in the art. For example, proteins may be extracted by ultra-centrifugation or other standard techniques. The separated proteins may be digested into peptides, in particular using proteolytic enzymes such as trypsin, pepsin, subtilisin, and proteinase. For example, proteins may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

Mass spectrometers that may be used to analyze the peptides or proteins include a Matrix-Assisted Laser Desorption/Ioniation Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

### Screening Methods

The invention contemplates a method of assessing the potential of a test compound to contribute to thyroid cancer comprising: (a) maintaining separate aliquots of thyroid cancer cells in the presence and absence of the test compound; and (b) comparing the levels of Thyroid Cancer Markers associated with the thyroid cancer in each of the aliquots. A significant difference between the levels of Thyroid Cancer Markers in an aliquot maintained in the presence of (or exposed to) the test compound relative to the aliquot maintained in the absence of the test compound, indicates that the test compound potentially contributes to thyroid cancer.

The invention also contemplates methods for evaluating test agents or compounds for their potential efficacy in treating thyroid cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides (e.g. antisense, siRNA), and small organic or inorganic molecules. The agents or compounds may be endogenous physiological compounds or natural or synthetic compounds.

The invention provides a method for assessing the potential efficacy of a test agent for potential efficacy in treating thyroid cancer in a patient the method comprising comparing: (a) levels of one or more Thyroid Cancer Markers, and optionally other markers of thyroid cancer, in a first sample obtained from a patient and exposed to the test agent; and (b) levels of one or more Thyroid Cancer Markers, and optionally other markers, in a second sample obtained from the patient, wherein the sample is not exposed to the test agent, wherein a significant difference in the levels of expression of one or more Thyroid Cancer Markers, and optionally the other markers, in the first sample, relative to the second sample, is an indication that the test agent is potentially efficacious for treating thyroid cancer in the patient. The first and second samples may be portions of a single sample obtained from a patient or portions of pooled samples obtained from a patient.

In an aspect, the invention provides a method of selecting an agent for treating thyroid cancer, in particular aggressive thyroid cancer in a patient comprising: (a) obtaining a sample from the patient; (b) separately maintaining aliquots of the sample in the presence of a plurality of test agents; (c) comparing one or more Thyroid Cancer Markers, and optionally other markers, in each of the aliquots; (d) selecting one of the test agents which alters the levels of one or more Thyroid Cancer Markers, and optionally other markers in the aliquot containing that test agent, relative to other test agents; and (e) optionally administering the selected test to the patient.

The invention further relates to a method of assessing the efficacy of a therapy for modulating thyroid cancer in a patient. A method of the invention comprises comparing: (a) levels of Thyroid Cancer Markers in a first sample from the patient obtained from the patient prior to providing at least a portion of the therapy to the patient; and (b) levels of Thyroid Cancer Markers in a second sample obtained from the patient following therapy. In an embodiment, a significant difference between the levels of Thyroid Cancer Markers in the second sample relative to the first sample or an abnormal state is an indication that the therapy is efficacious for modulating the thyroid cancer. In a particular embodiment, the method is used to assess the efficacy of a therapy for treating a thyroid cancer where lower levels of Thyroid Cancer Markers in the second sample relative to the first sample, is an indication that the therapy is efficacious. The “therapy” may be any therapy for treating thyroid cancer including but not limited to therapeutics, gene therapy, and surgery. Therefore, the method can be used to evaluate a patient before, during, and after therapy.

The invention contemplates a method for determining the effect of an environmental factor on thyroid cancer comprising comparing Thyroid Cancer Markers in the presence and absence of the environmental factor.

### Kits

The invention contemplates kits for carrying out the methods of the invention to diagnosis thyroid cancer or stage of thyroid cancer, and to detect the aggressiveness or metastatic potential of a thyroid cancer. Such kits typically comprise two or more components required for performing a diagnostic assay. Components include but are not limited to compounds, reagents, containers, and/or equipment. Accordingly, the methods described herein may be performed by utilizing pre-packaged test or diagnostic kits comprising at least agents (e.g. antibodies, probes, primers, etc) described herein, which may be conveniently used, e.g., in clinical settings, to diagnose patients, in particular patients afflicted with thyroid cancer, suspected of having thyroid cancer, or at risk of thyroid cancer or exhibiting a predisposition to developing thyroid cancer, and more particularly to determine the aggressiveness or metastatic potential of a thyroid cancer.

The invention contemplates a kit with a container comprising a binding agent(s) as described herein for characterizing a thyroid cancer. By way of example, the kit may contain antibodies specific for a Polypeptide Thyroid Cancer Marker(s), antibodies against the antibodies labelled with an enzyme(s), and a substrate for the enzyme(s). The kit may also contain microtiter plate wells, standards, assay diluent, wash buffer, adhesive plate covers, and/or instructions for carrying out a method of the invention using the kit.

In an aspect, the invention provides a test kit for diagnosing thyroid cancer in a subject which comprises an antibody that binds to a Polypeptide Thyroid Cancer Marker(s) and/or polynucleotides that hybridize to or amplify Polynucleotide Thyroid Cancer Marker(s). In another aspect the invention relates to use of an antibody that binds to a Polypeptide Thyroid Cancer Marker and/or a polynucleotide that hybridizes to or amplifies a Polynucleotide Thyroid Cancer Marker, in the manufacture of a composition for detecting or characterizing a thyroid cancer.

In a further aspect of the invention, the kit includes antibodies or antibody fragments which bind specifically to epitopes of Polypeptide Thyroid Cancer Marker(s) and means for detecting binding of the antibodies to their epitopes associated with thyroid cancer cells, either as concentrates (including lyophilized compositions), which may be further diluted prior to testing. In particular, the invention provides a kit for diagnosing or characterizing thyroid cancer comprising a known amount of a first binding agent that specifically binds to a Polypeptide Thyroid Cancer Marker(s) wherein the first binding agent comprises a detectable substance, or it binds directly or indirectly to a detectable substance.

A kit may be designed to detect the levels of Polynucleotide Thyroid Cancer Markers in a sample. Such kits generally comprise oligonucleotide probes or primers, as described herein, which hybridize to or amplify Polynucleotide Thyroid Cancer Markers. Oligonucleotides may be used, for example, within PCR or hybridization procedures. Test kits useful for detecting target Polynucleotide Thyroid Cancer Markers are also provided which comprise a container containing a Polynucleotide Thyroid Cancer Marker, and fragments or complements thereof.

The kits of the invention can further comprise containers with tools useful for collecting test samples (e.g. serum) including lancets and absorbent paper or cloth for collecting and stabilizing blood.

### Computer Systems

Analytic methods contemplated herein can be implemented by use of computer systems and methods described below and known in the art. Thus, the invention provides computer readable media comprising one or more Thyroid Cancer Markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more markers disclosed herein.

A variety of data processor programs and formats can be used to store information on one or more Thyroid Cancer Markers. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has thyroid cancer, in particular aggressive thyroid cancer, or a pre-disposition to such condition, comprising determining the presence or absence of one or more Thyroid Cancer Markers, and based on the presence or absence of the markers, determining the condition or a pre-disposition to the condition, optionally recommending a procedure or treatment.

The invention also provides in an electronic system and/or in a network, a method for determining whether a subject has a condition disclosed herein, or a pre-disposition to a condition disclosed herein, comprising determining the presence or absence of one or more markers, and based on the presence or absence of the markers, determining whether the subject has the condition or a pre-disposition to the condition, and optionally recommending a procedure or treatment.

The invention further provides in a network, a method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from the network corresponding to the markers; and (c) based on the phenotypic information and information on the markers, determining whether the subject has the condition or a pre-disposition to the condition, and (d) optionally recommending a procedure or treatment.

The invention still further provides a system for identifying selected records that identify a diseased cell or tissue. A system of the invention generally comprises a digital computer; a database server coupled to the computer; a database coupled to the database server having data stored therein, the data comprising records of data comprising one or more markers disclosed herein, and a code mechanism for applying queries based upon a desired selection criteria to the data file in the database to produce reports of records which match the desired selection criteria.

The invention contemplates a business method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from a network corresponding to the markers; and (c) based on the phenotypic information, information on the markers and acquired information, determining whether the subject has the condition or a pre-disposition to the condition, and optionally recommending a procedure or treatment.

In an aspect of the invention, the computer systems, components, and methods described herein are used to monitor a condition or determine the stage of a condition.

### Therapeutic Applications

The invention contemplates therapeutic applications associated with the Thyroid Cancer Markers disclosed herein including thyroid cancer. Thyroid Cancer Markers may be a target for therapy. For example, markers in Table 1 can be a target for treatment of thyroid cancers.

Therapeutic methods include immunotherapeutic methods including the use of antibody therapy. In one aspect, the invention provides one or more antibodies that may be used to prevent thyroid cancer. In another aspect, the invention provides a method of preventing, inhibiting or reducing thyroid cancer comprising administering to a patient an antibody which binds to a Thyroid Cancer Marker in an amount effective to prevent, inhibit, or reduce the condition or the onset of the condition. The invention also contemplates a method of treating thyroid cancer in a subject, comprising delivering to the subject in need thereof, an antibody specific for a Thyroid Cancer Marker in Table 1, in particular Thyroid Cancer Marker in Table 1 that is upregulated in thyroid cancer or a stage of thyroid cancer. According to one aspect of the invention, there is provided a method of treating a subject having thyroid cancer wherein an antibody specific for a marker in Table 1 is administered in a therapeutically effective amount. In a further aspect, the antibody is provided in a pharmaceutically acceptable form.

An antibody which binds to a Thyroid Cancer Marker may be in combination with a label, drug or cytotoxic agent, a target-binding region of a receptor, an adhesion molecule, a ligand, an enzyme, a cytokine, or a chemokine. In aspects of the invention, the Thyroid Cancer Marker may be conjugated to cytotoxic agents (e.g., chemotherapeutic agents) or toxins or active fragments thereof. Examples of toxins and corresponding fragments thereof include diptheria A chain, exotoxin A chain, ricin A chain, abrin A chain, curcin, crotin, phenomycin, enomycin and the like. A cytotoxic agent may be a radiochemical prepared by conjugating radioisotopes to antibodies, or binding of a radionuclide to a chelating agent that has been covalently attached to the antibody. An antibody may also be conjugated to one or more small molecule toxins, such as a calicheamicin, a maytansine, a trichothene, and CC1065 (see U.S. Pat. No. 5,208,020).

The methods of the invention contemplate the administration of single antibodies as well as combinations, or “cocktails”, of different individual antibodies such as those recognizing different epitopes of other markers. Such cocktails may have certain advantages inasmuch as they contain antibodies that bind to different epitopes of Thyroid Cancer Markers and/or exploit different effector mechanisms. Such antibodies in combination may exhibit synergistic therapeutic effects. In addition, the administration of one or more marker specific antibodies may be combined with other therapeutic agents. The specific antibodies may be administered in their “naked” or unconjugated form, or may have therapeutic agents conjugated to them.

In an aspect, the invention provides a pharmaceutical composition for the treatment of thyroid cancer characterized in that the composition comprises an antibody specific for a marker in Table 1, in particular a Thyroid Cancer Marker that is upregulated in thyroid cancer or a type of thyroid cancer, together with a pharmaceutically acceptable carrier, excipient or vehicle.

Antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

One or more marker specific antibody formulations may be administered via any route capable of delivering the antibodies to the site or injury. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the condition, stage of the condition, the binding affinity and half life of the antibodies used, the degree of marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more marker antibodies is also possible and may have advantages in certain situations.

Patients may be evaluated for Thyroid Cancer Markers in order to assist in the determination of the most effective dosing regimen and related factors. The assay methods described herein, or similar assays, may be used for quantitating marker levels in patients prior to treatment. Such assays may also be used for monitoring throughout therapy, and may be useful to gauge therapeutic success in combination with evaluating other parameters such as levels of markers.

Polynucleotide Thyroid Cancer Markers disclosed herein can be turned off by transfecting a cell or tissue with vectors that express high levels of the polynucleotides. Such constructs can inundate cells with untranslatable sense or antisense sequences. Even in the absence of integration into the DNA, such vectors may continue to transcribe RNA molecules until all copies are disabled by endogenous nucleases. Vectors derived from retroviruses, adenovirus, herpes or vaccinia viruses, or from various bacterial plasmids, may be used to deliver polynucleotides to a targeted organ, tissue, or cell population. Methods well known to those skilled in the art may be used to construct recombinant vectors that will express polynucleotides such as antisense. (See, for example, the techniques described in Sambrook et al (supra) and Ausubel et al (supra).)

Methods for introducing vectors into cells or tissues include those methods known in the art which are suitable for in vivo, in vitro and ex vivo therapy. For example, delivery by transfection or by liposomes is well known in the art.

Modifications of gene expression can be obtained by designing antisense molecules, DNA, RNA or PNA, to the regulatory regions of a Polynucleotide Thyroid Cancer Marker, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of a polynucleotide marker.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

In some aspects one or more Polypeptide Thyroid Cancer Markers and polynucleotides encoding the markers, and fragments thereof, may be used in the treatment of a thyroid cancer in a subject. In an aspect the Thyroid Cancer Marker is down-regulated in thyroid cancer. The markers may be formulated into compositions for administration to subjects suffering from a thyroid cancer. Therefore, the present invention also relates to a composition comprising one or more Thyroid Cancer Markers, preferably a Thyroid Cancer Marker downregulated in thyroid cancer, and a pharmaceutically acceptable carrier, excipient or diluent. A method for treating or preventing a thyroid cancer in a subject is also provided comprising administering to a patient in need thereof, one or more one or more Polypeptide Thyroid Cancer Markers and polynucleotides encoding the markers, or a composition of the invention.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

A composition described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999. On this basis, the compositions include, albeit not exclusively, solutions of the active substances in association with one or more pharmaceutically acceptable vehicles or diluents, and contained in buffered solutions with a suitable pH and iso-osmotic with the physiological fluids.

A composition is indicated as a therapeutic agent either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting examples are illustrative of the present invention:

### EXAMPLE 1

Proteins that are secreted by cultured cancer cells into the media of their cell culture plates (i.e. “secretome” proteins) make especially appealing targets for study because they may be detectable in bodily fluids. The study described in this example examines the secretome of seven thyroid cancer cell lines: TPC-1, BCPAP, CAL 62, SW1736, C643, MRO, and WRO. Proteomic analysis of the conditioned serum-free media of these cells using LC-MS/MS allows for identification of proteins that these cancer cells secrete. This serves as a surrogate for proteins that human thyroid cancer cells secrete in vivo. Identification of secretome proteins has lead to the discovery of numerous potential thyroid cancer biomarkers that may be used to predict aggressiveness of thyroid cancers. Furthermore, the study independently validates selected secretome proteins in the sera of thyroid cancer patients versus cancer-free individuals using Western blots.

The following materials and methods were employed in the Study described in this Example.

### Materials and Methods

Cell Culture and Serum Free Media Collection. Seven thyroid cancer cell lines are used in this study TPC-1 (papillary), BCPAP (papillary), CAL62 (anaplastic), SW1736 (anaplastic), C643 (anaplastic), MRO (follicular), and WRO (follicular). The cells were grown in 25 mL of conditioned RPMI-1640 cell culture media (containing antibodies and supplemented with 10% fetal bovine serum) in 150 mm dishes to approximately 65% confluence. Cells were kept at 37° C. in a humidified atmosphere of 5% CO₂/95% air. The conditioned media was then aspirated and cells were washed three times with phosphate-buffered saline (PBS). Thereafter, cells were washed once with serum-free media (SFM) that was collected as a time 0 h control. Cells were incubated in the SFM for 48 hours. Following 48 h, the SFM was collected, centrifuged at 2200 RPM for 5 minutes at 4° C., and filtered using a 0.2 μm nylon filter. Upon filtration collected SFM samples were immediately frozen at −80° C. until later processing. SFM was collected from sixty 150 mm plates for TPC-1, SW1736 and CAL 62, and from twenty-five 150 mm BCPAP, C643, WRO and MRO plates.
Protein Precipitation from collected SFM and Preparation for LC-MS/MS analysis. Proteins were isolated from SFM using 0.2% sodium deoxycholate (Sigma Aldrich, MO) and 10% trichloroacetic acid (Sigma Aldrich, MO) as described earlier. [4] Following 2 h incubation on ice, the samples were centrifuged at 11 000 g for 30 minutes and washed two times with ice-cold acetone. The precipitated proteins were then dissolved in 50 mM NaHCO₃ buffer. Protein concentration was later determined using the Bradford assay (Bio-Rad, CA). Protein samples were then heated for 1 h at 65° C. in the presence of 5 mM dithiothreitol, cooled to room temperature, and incubated in the dark for 1 h with 10 mM iodoacetamide to allow for alkylation. Sequencing grade trypsin (Promega, WI) at 1:20 (w/w) in 50 mM ammonium bicarbonate was subsequently added and the samples were incubated at 37° C. overnight. Digested samples were then dried under vacuum and redissolved in 10 μL of 0.1% formic acid.
Liquid Chromatography-MS/MS analysis. Samples were analyzed by online LC-MS/MS in triplicates. The nanobore LC system and MS/MS setup was followed and has been described earlier [5]. The liquid chromatograph used in the experiment was an LC Packings Ultimate unit (Amsterdam, The Netherlands). The mass spectrometer used was a QSTAR Pulsar-i hybrid quadrupole/time-of flight (QqTOF) instrument (Applied Biosystems/MDS SCIEX, CA). An autosampler was used to load 1 μL of sample onto a C18 reverse-phase precolumn (LC Packings: 300 μm×5 mm). Subsequently, reverse-phase chromatography on an analytical column (75 μm×150 mm packed in-house with 3-μm Kromasil C18 beads with 100 Å pores, The Nest Group) was used. For separation, a nonlinear binary gradient was used: eluant A consisting of 94.9% deionized water, 5.0% acetonitrile, and 0.1% formic acid (pH 3); and eluant B consisting of 5.0% deionized water, 94.9% acetonitrile, and 0.1% formic acid. During the first 5 min of the LC run, eluant A at a flow rate of 25 μL min⁻¹ was used to load peptides from the sample onto the C18 precolumn. Desalting continued for two additional min. At the 8^{th} min, the C18 precolumn was switched inline with the reverse-phase analytical column; separation was performed at 200 nL min⁻¹ using a 180-min binary gradient shown below.

| Time (min) | 0 | 5 | 10 | 120 | 140 | 145 | 155 | 157 | 189 |
|---|---|---|---|---|---|---|---|---|---|
| B (%) | 5 | 5 | 15 | 35 | 60 | 80 | 80 | 5 | Stop |

MS/MS Settings and Data Collection. Data was collected in information-dependent acquisition (IDA) mode using Analyst QS 1.1 and Bioanalyst Extension 1.1 software (Applied Biosystems/MDS SCIEX). MS cycles consisted of a TOF MS survey scan with an m/z range of 400-1500 Th for 1 s. This was followed by five product-ion scans with an m/z range of 80-2000 Th for 2 s each. IDA CE Parameters script was used to control the collision energy (CE). Switching criteria were set to ions with m/z≧400 and ≦1500 Th, charge states of 2-4, and abundances of ≧10 counts. Former target ions were excluded for 30 s, and ions within a 6-Th window were ignored. Additionally, the IDA Extensions II script was set to “no repetition” before dynamic exclusion and to select a precursor ion nearest to a threshold of 10 counts on every fourth cycle. LC-MS/MS data were searched using the ProteinPilot software (Applied Biosystems, Foster City, Calif.) and a Celera human protein database (CDS KBMS 20041109) containing 178239 protein sequences. The cutoff for significance used for this search was set for a score of 1.3, which corresponds to a confidence score of 95%.
Secretion features of identified proteins. To analyze identified proteins' secretion features, Signal Peptide Predictor (SignalP, http://www.cbs.dtu.dk/services/SignalP 3.0) was used. SignalP uses amino-acid sequences to predict the existence and location of signal peptide cleavage sites. SignalP determines the likelihood a protein is a signaling peptide by using numerous artificial neural networks and hidden Markov model algorithms to detect signal peptides from protein sequences. A protein is considered classically secreted if it receives a signal peptide probability≧0.900. In order to identify non-classical, or leaderless, protein secretion SecretomeP (http://www.cbs.dtu.dk/services/SecretomeP 2.0) was used. SecretomeP uses a neural network that combines six protein characteristics to determine if a protein is non-classically secreted. The protein characteristics include: the number of atoms, number of positively charged residues, presence of transmembrane helices, presence of low-complexity regions, presence of pro-peptides, and subcellular localization. A protein is considered non-classically secreted if it receives an NN-score≧0.500 (note: only proteins that were not considered classically secreted, i.e. received SignalP scores<0.900, were analyzed using SecretomeP).
Western Blot: Verification of Biomarkers in Sera. Western Blots were used to verify the expression of selected secretory proteins, E-cadherin, Nucleolin, CYR61 (cysteine rich angiogenic inducer, 61 variant), Prothomyosin alpha, □-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET and calsyntenin-1 in thyroid cancer patients' blood. Patient serum samples were depleted of the 20 most abundant blood proteins using the Proteoprep 20 Plasma Immunodepletion kit (Sigma-Aldrich, MO) according to manufacturer's specifications.

For Western Blot analysis, 12% SDS-PAGE gels were used as described earlier. Proteins were transferred from the gel to a polyvinylidenedifluoride (PVDF) membrane, that was blocked using 5% non-fat milk in Tris-buffered saline (TBS, 0.1 M, pH=7.2). Blots were incubated using monoclonal or polyclonal antibodies against E-cadherin, Nucleolin, CYR61 (cysteine rich angiogenic inducer, 61 variant), Prothomyosin alpha, □-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET and calsyntenin-1 at the appropriate dilutions at 4° C. for 2 hours. The membranes were incubated with horseradish peroxidase-conjugated anti-rabbit or anti-mouse secondary antibody (DAKO Cytomation, Denmark), diluted to an appropriate concentration in 1% BSA, and room-temperature incubated for 2 h. Following each step, blots were washed three times with Tween (0.1%)-Tris-buffer saline (TTBS). Protein bands were detected by the enhanced chemiluminescence method (ECL, Santa Cruz Biotechnology, CA) on XO-MAT film.

### Results

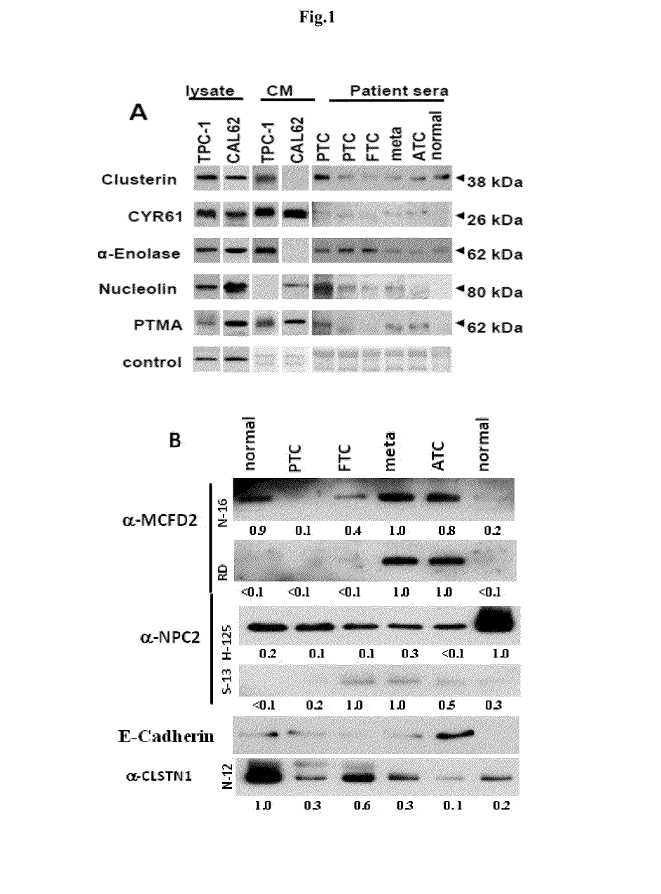

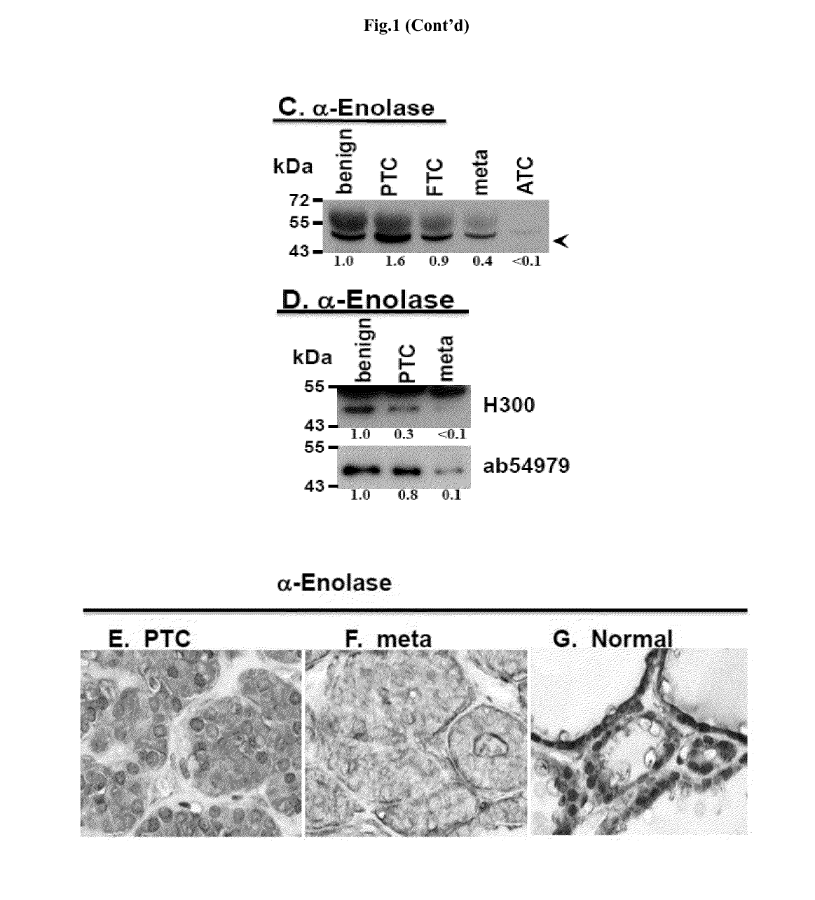

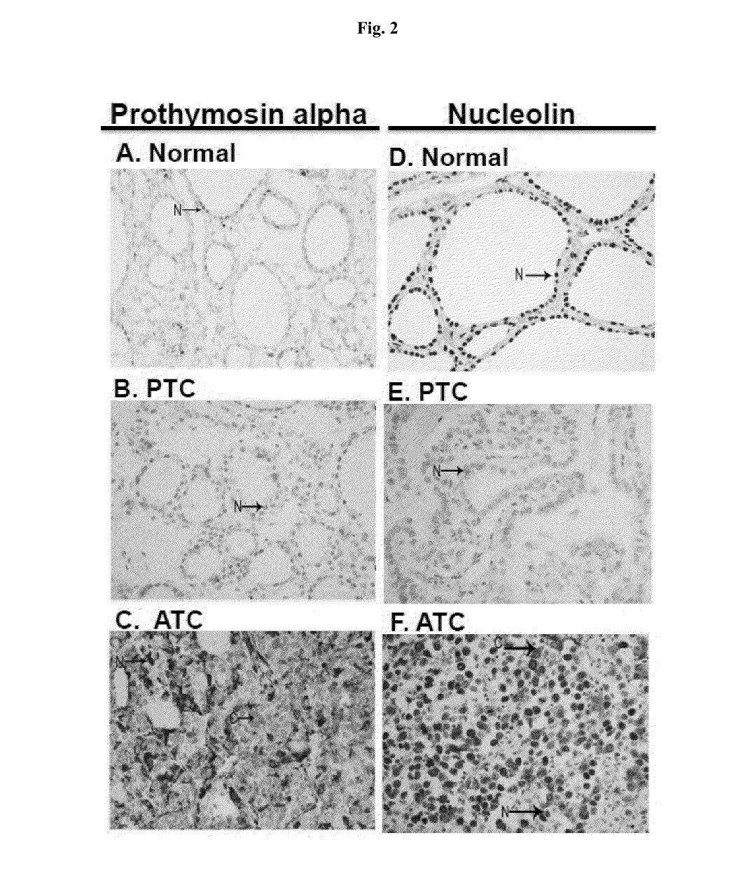

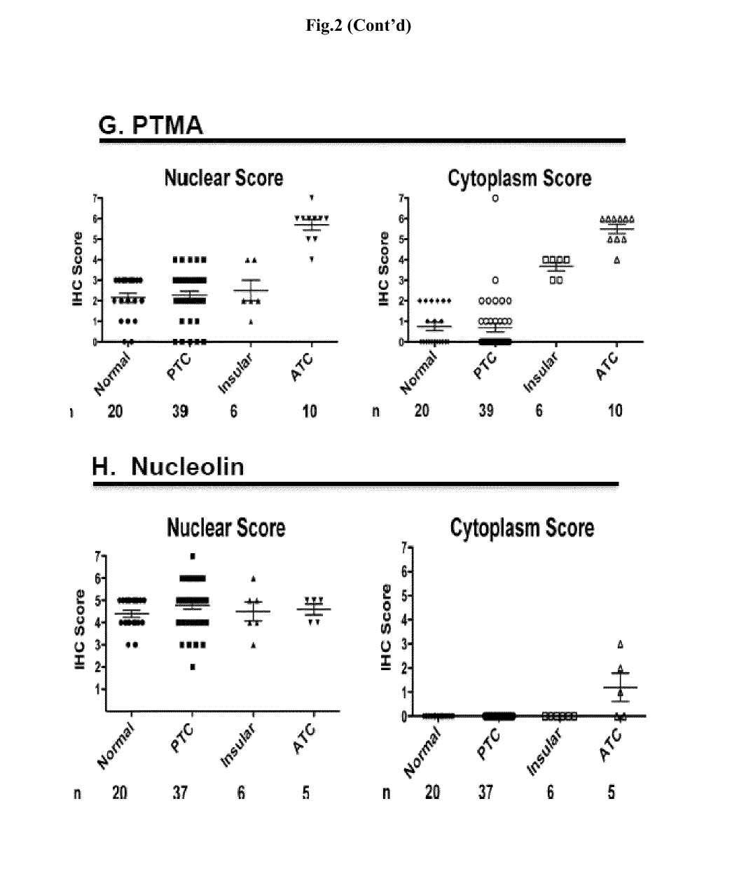

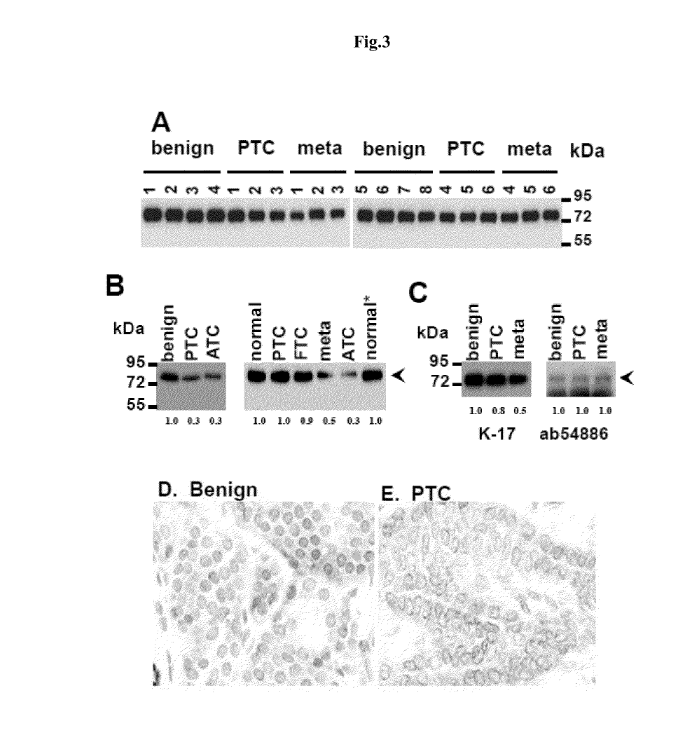

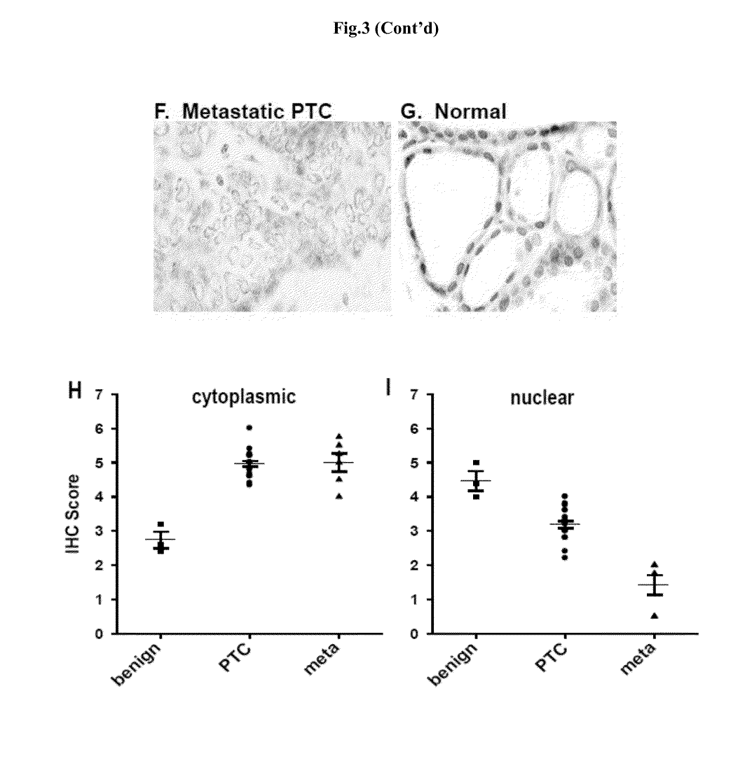

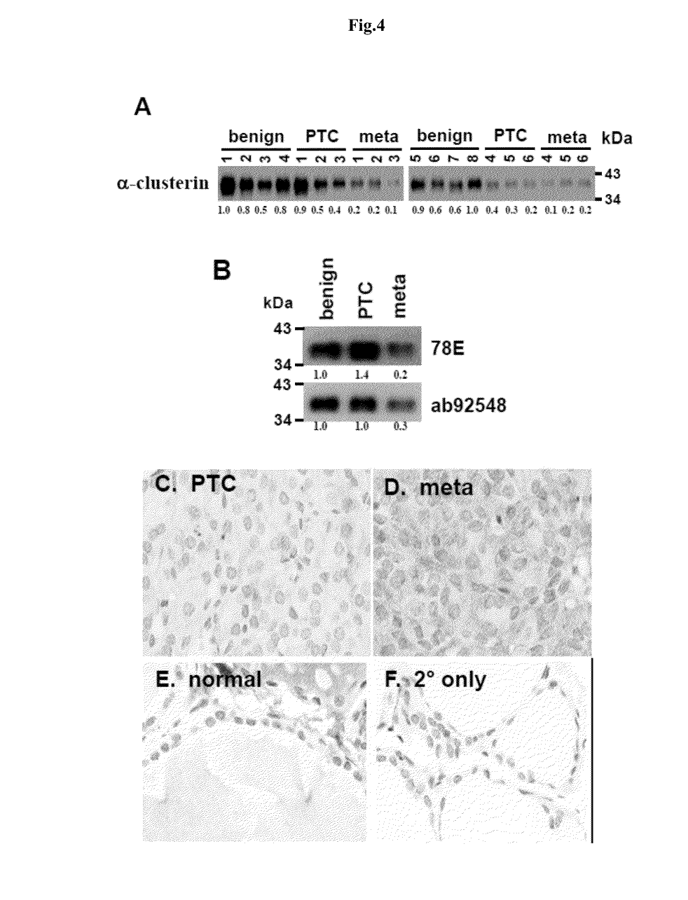

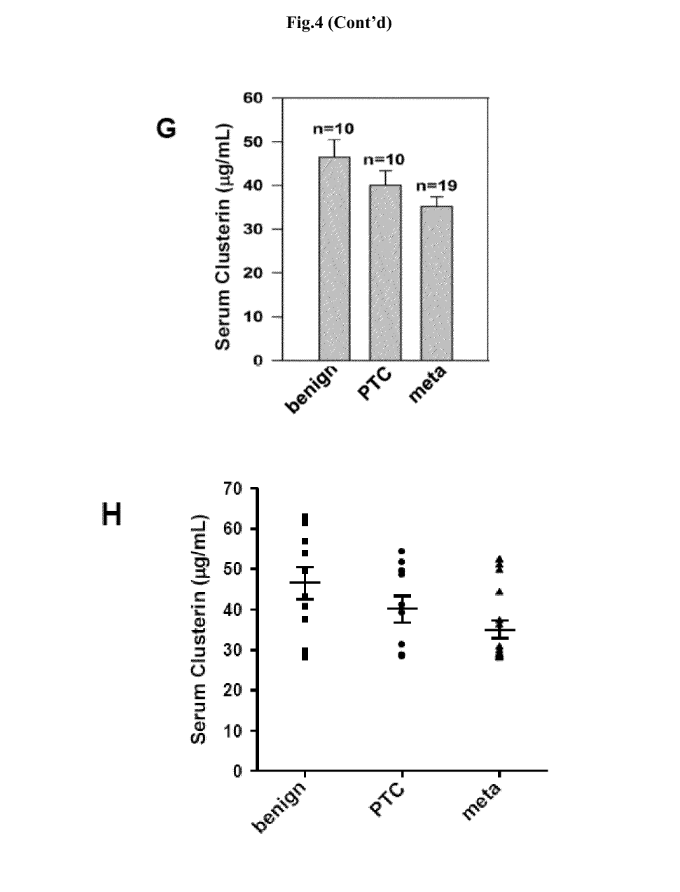

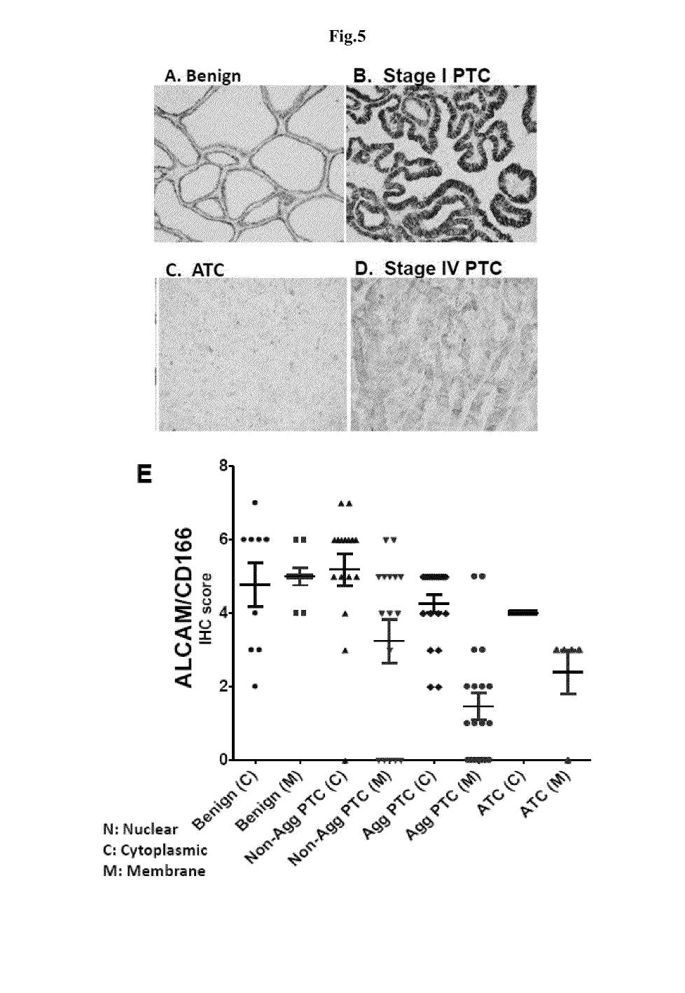

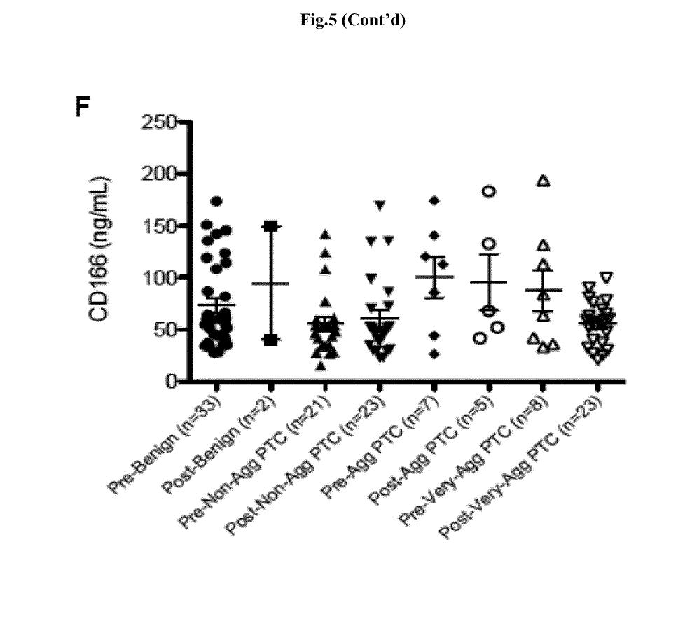

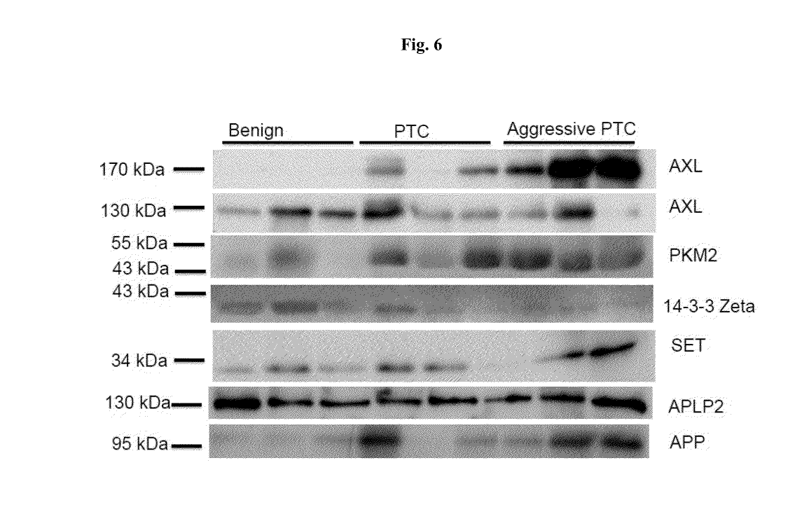

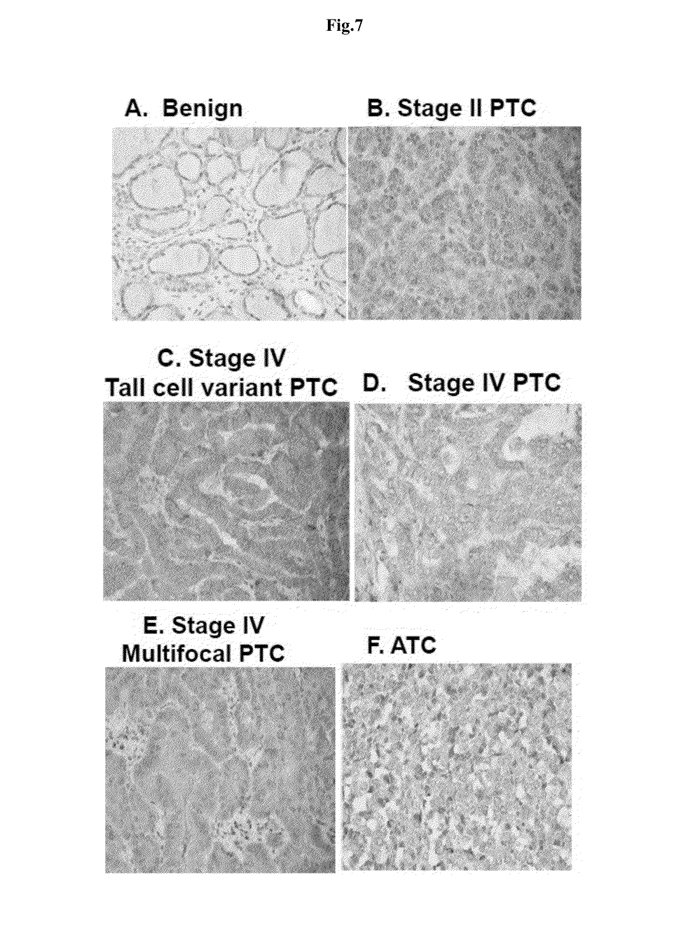

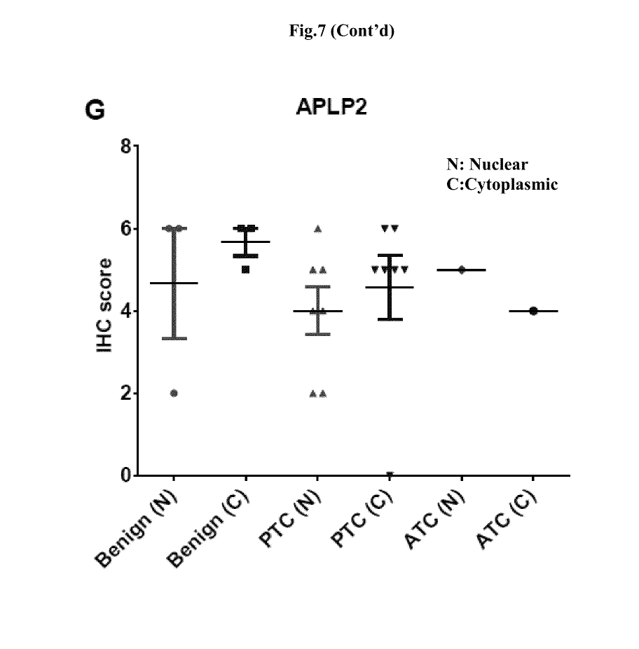

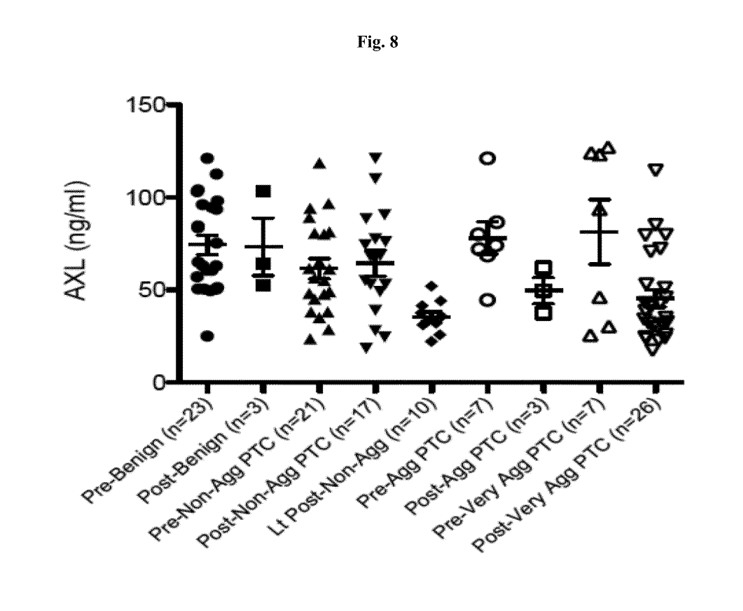

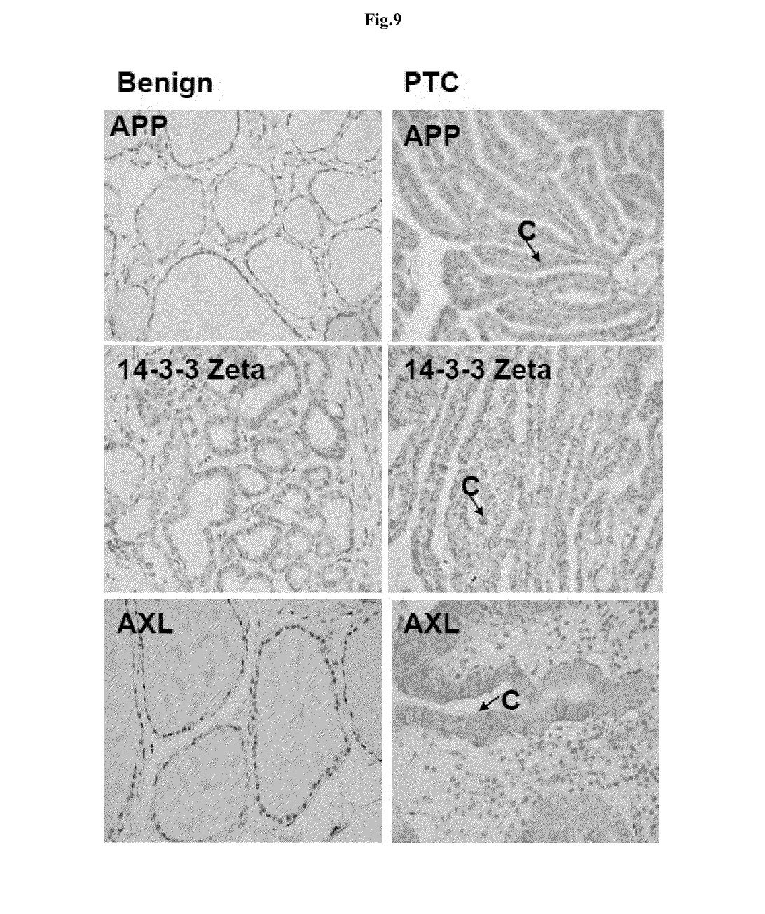

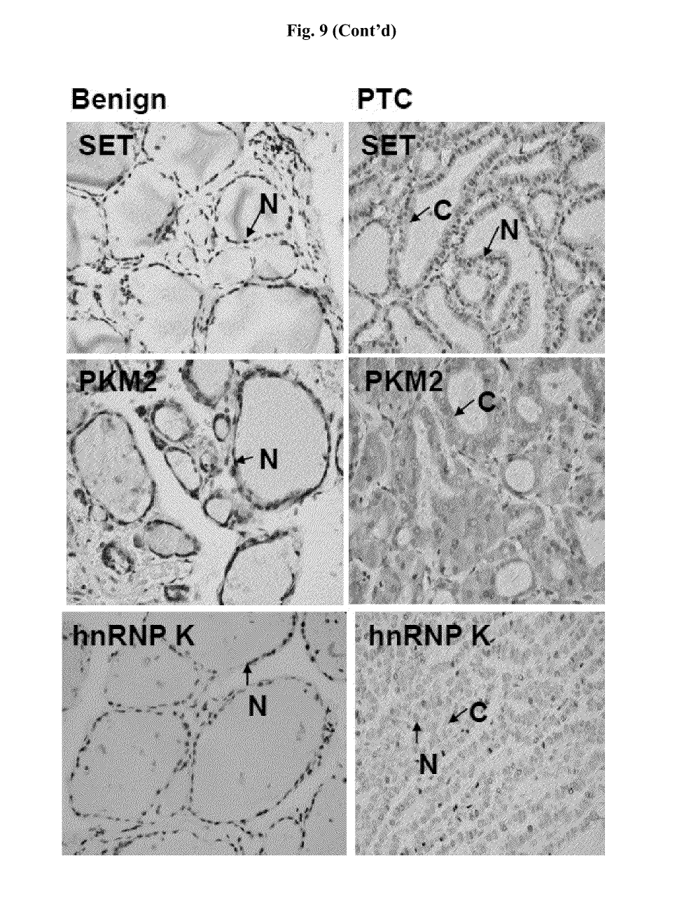

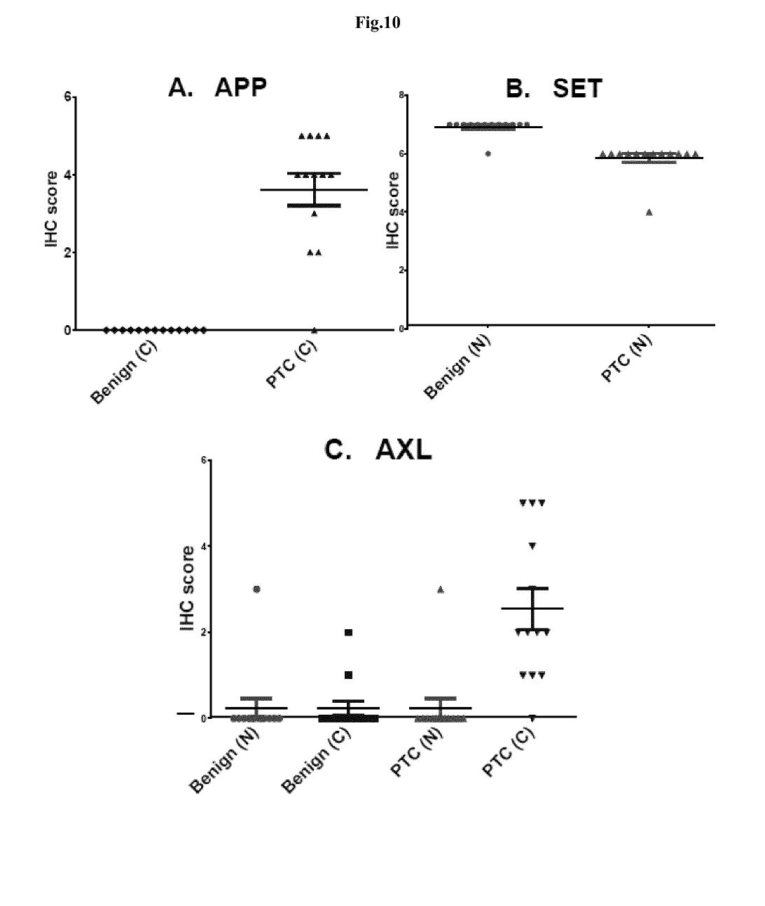

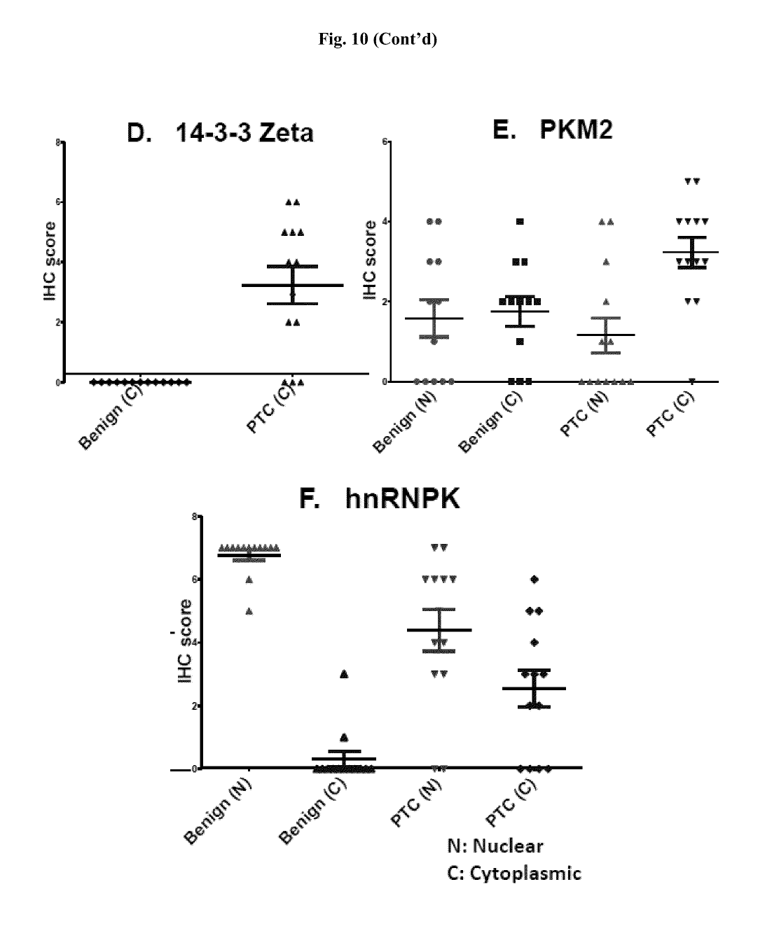

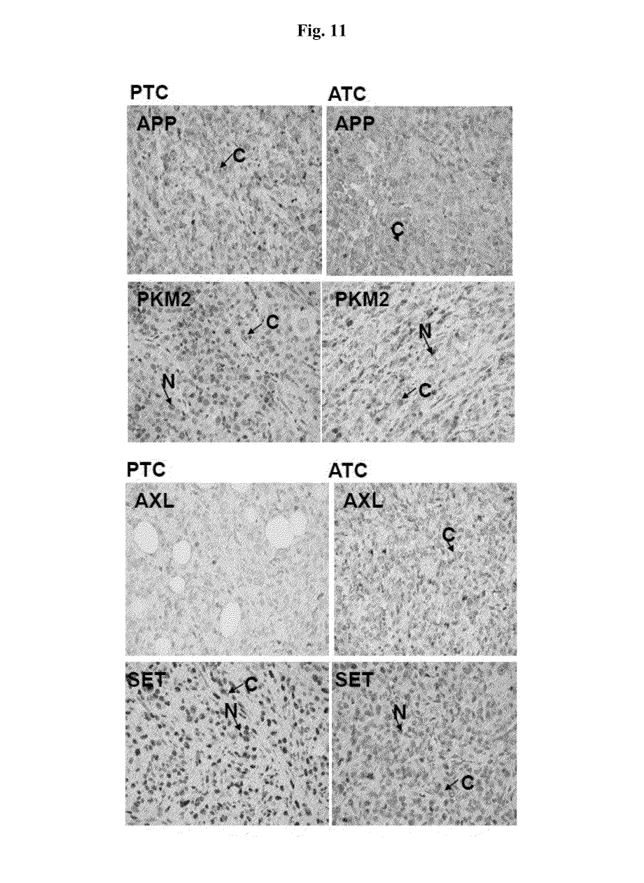

The results are discussed below and aspects are illustrated in FIGS. 1-11.
Optimization of Cell Culture Conditions for SFM Collection. While cells are normally grown in media that contains serum, the high abundance proteins found in serum would interfere with the detection of secretome proteins. For this reason, cell culture conditions needed to be optimized for SFM collection. To avoid this interference, the cells were washed thoroughly four times (three times with PBS and once with serum-free media) and then grown in serum-free media for 48 h, allowing secretome proteins to accumulate. To limit cellular stress under these conditions, cells were only placed in SFM when they reached 60% confluence. Trypan blue staining was performed following collection of the SFM at 48 h to estimate the number of dead cells. Since >95% of cells were viable at 48 h, the release of non-secretory proteins into the media is considered to be minimal, but cannot be completely ruled out.
Proteins Released by TPC-1, CAL 62, MRO, WRO, BCPAP, SW1736 and C643 Thyroid Cancer Cell Lines. A total of 233 proteins were initially identified in the four thyroid cancer cell lines. The subcellular localization and biological functions of the proteins were determined using Ingenuity Pathway Analysis (IPA, Ingenuity Systems, www.ingenuity.com). In all cell lines, membrane and extracellular proteins were predominantly identified. Additionally, proteins associated with cellular metabolism were common to all cell lines. Numerous signal transduction and cell cycle proteins were also identified in WRO and TPC-1 cells. In order to become a candidate for further verification, proteins must have been identified from MS spectrum data with at least 2 high-confidence peptides with a confidence level≧95%. Proteins identified with at least two high-confidence peptides are considered high-confidence identifications. Proteins were not identified from the 0 h controls, except for blood albumin and globulins, which were removed from the identified proteins list. After applying the high confidence threshold to the identified protein list, 83 proteins remained as candidates for independent verification (see Table 1). Protein sequences were obtained for these proteins and inputted into SignalP and/or SecretomeP to obtain the reported score. Literature searches were performed on each protein to identify its cellular localization, and whether it has been reported to be present in exosomes or in patient blood/tissue samples. Nearly all of these high-confidence identifications were determined to be secretory proteins according to their SignalP and SecretomeP scores.
Verification of Selected Secretome Proteins in Human Sera by Western Blotting. The presence of select proteins were independently verified by Western Blot in thyroid cancer patients' sera, and in the SFM (see FIGS. 1A-D, 3A-C, 4A,B and 6). The proteins that have been verified are E-cadherin, Nucleolin, CYR61 (cysteine rich angiogenic inducer, 61 variant), Prothomyosin alpha, □-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET, and calsyntenin-1. Proteins were selected for verification were based upon SecretomeP scores, SignalP scores, IPA database information, along with information from literature searches (i.e. whether the protein has also been reported to be present in the blood of thyroid cancer patients in other studies, whether it has been reported to be found in exosomes, and/or the role the protein may play in disease progression). The justification for selection of these proteins lies in the fact that they were high-confidence identifications, are suggested to be secreted proteins based upon their SignalP and/or SecretomeP scores, and/or have been reported in the literature to be secreted or to play a role in cancer pathogenesis.
Verification of Selected Secretome Proteins in Human Sera by ELISA. The presence of select proteins were independently verified by ELISA in thyroid cancer patients' sera (see FIGS. 4G and H, 5F and 8). The proteins that have been verified are clusterin, ALCAM/CD166 and AXL tyrosine kinase.
Verification of Selected Secretome Proteins in Human Thyroid Carcinoma and Normal Tissues by Immunohistochemistry. The presence of select proteins were independently verified by immunohistochemistry in thyroid carcinoma, benign thyroid nodules and/or normal tissues (see FIGS. 1E-G, 2A-H, 3D-I, 4C-F, 5A-E, 7A-G, 9 and 10). The proteins that have been verified are □-Enolase, Prothomyosin alpha, Nucleolin, Biotinidase, Clusterin, ALCAM/CD166, amyloid precursor protein like protein 2 (APLP2), amyloid precursor protein (APP), 14-3-3 zeta, Tyrosine-protein kinase receptor UFO (AXL), SET, Pyruvate kinase M2 (PKM2), and Heterogeneous ribonucleoprotein K (hnRNP K).
Verification of Selected Secretome Proteins in Mouse Xenografts of Human Thyroid Carcinoma Cell Lines. The presence of select proteins were independently verified by immunohistochemistry in tissue sections of xenografts of human thyroid carcinoma cell lines, BCPAP (papillary thyroid carcinoma) and C643 (anaplastic thyroid carcinoma) in immunocompromised mice (NOD/Scid/gamma) (see FIG. 11). The proteins that have been verified are amyloid precursor protein (APP), Tyrosine-protein kinase receptor UFO (AXL), Pyruvate kinase M2 (PKM2), and SET. The expression patterns and subcellular localization of these proteins in mouse xenografts were similar to those observed in cultured thyroid cancer cells and in human thyroid carcinomas confirming that these proteins retain their characteristics in xenografts.

### Discussion

The study revealed a total of 233 proteins in the secretome of TPC-1, BCPAP, CAL 62, SW1736, WRO, and MRO cells, of which 83 are considered high-confidence identifications due to the numerous peptides used in their identification. Nearly all identified high-confidence proteins were deemed to be secretory according to their SignalP and SecretomeP scores, lending additional support to the hypothesis that the proteins identified in the study are secretory proteins. There were far more proteins unique to TPC-1 and SW1736 cells than the other cell lines due to the fact that three-times as many TPC-1 and SW1736 cells were used for SFM collection.
Nucleolin. Nucleolin is a nuclear protein involved in numerous cell cycle processes. It does not have a known classical secretion signal and is not suggested to be a secretory protein based upon its SecretomeP and SignalP scores. SecretomeP and SignalP scores cannot completely rule out the possibility a protein is in-fact secretory, and numerous studies have demonstrated that nucleolin is in-fact, present on the cellular surface of proliferating cells [6]. It remains unclear how nucleolin is transported from the nuclear membrane to the cell surface. It has been shown that the use of antagonists to surface nucleolin suppresses tumour growth and angiongenesis, suggesting an important role between cell-surface nucleolin expression and tumour progression [7]. Confirmatory western blotting revealed nucleolin to be present in all of the cell lysates, but only in the SFM of WRO and MRO cells. It was also detectable in all 5 thyroid cancer patient samples, but not in the normal blood sample. The detectablity of nucleolin in patient blood samples suggests it may be a useful thyroid cancer biomarker.
Cysteine Rich Angiogenic Inducer 61 (CYR61). CYR61 belongs to the CCN family of proteins, initially identified as secretory proteins whose production is induced by oncogenes [8]. Paradoxically, CYR61, while having demonstrated importance in cancer cell proliferation, has also been shown to play an important role in the induction of apotosis [9]. The secretome analysis revealed CYR61 to be secreted by TPC-1 cells. As with nucleolin, it was present in all thyroid cancer patient blood samples, but not in the normal. Interestingly, CYR61 was only found in the whole cell lysate of TPC-1 cells. This illustrates the potential for secretome analysis to reveal markers that may be used to distinguish between different thyroid cancer types.
E-Cadherin. The cadherins are a family of proteins responsible for cell-cell adhesion. Studies have shown that loss of E-cadherin mediated cell adhesion is associated with increased tumour aggressiveness and patient mortality [10]. E-Cadherin expression was noticed in the cell lysate and SFM of WRO and MRO cells, but not TPC-1. It was also present in all thyroid cancer blood samples, but not in the normal controls.
Prothymyosin alpha. Prothymyosin alpha is a heterochromatin remodeling protein whose expression has previously been shown to be significantly elevated in well-differentiated thyroid carcinomas compared to ademonas and goitres. [11] While it was present in the cell lysate of all three cell-lines, it was found to be secreted only in TPC-1 cells. As with nucleolin, CYR61, and E-Cadherin, prothymyosin alpha may serve as a potential thyroid cancer biomarker as it was found in all thyroid cancer patient blood samples but not in the normal controls.
Activated leukocyte cell adhesion molecule (ALCAM/CD166). Activated leukocyte cell adhesion molecule (ALCAM/CD166) is usually expressed in cells that are involved in growth and migration, including neural development, immune response, and tumor formation. [12, 13] It is an adhesion molecule that is located at intercellular junctions and is involved in tumor cell adhesion, which is necessary for primary tumor formation and metastasis. ALCAM binds to CD6 on T-cells and mediates T-cell activation and proliferation. ALCAM was identified in four thyroid cancer cell lines, including TPC-1, BCPAP, CAL62, and SW1736.
AXL. AXL is a receptor tyrosine kinase, ubiquitously expressed transmembrane protein, that binds to growth factors and transduces signals from the extracellular matrix to the cytoplasm. It is involved in stimulating cell proliferation and aggregation through hemophilic binding. AXL overexpression plays a role in cell adhesion and overexpression of this protein has been found in several cancers. [14]
APP. Amyloid beta (A4) protein is a cell surface receptor and transmembrane precursor protein that is cleaved by different secretases to form a variety of peptides which can bind to complexes for transcriptional activation. APP plays a role in development of the adult nervous system, cell adhesion, neuronal survival, neurite outgrowth, synaptogenesis, vesicular transport, neuronal migration, modulation of synaptic plasticity, and insulin and glucose homeostasis. [15]
PKM2. Normal cells express the pyruvate kinase M1 isoform (PKM1), tumor cells predominantly express the M2 isoform (PKM2). Switching from PKM1 to PKM2 promotes aerobic glycolysis and provides a selective advantage for tumor formation. The PKM1/M2 isoforms are generated through alternative splicing of two mutually exclusive exons. A recent study shows that the alternative splicing event is controlled by heterogeneous nuclear ribonucleoprotein (hnRNP) family members hnRNPA1, hnRNPA2, and polypyrimidine tract binding protein (PTB; also known as hnRNPI). [16]
APLP2. Amyloid-like protein 2 (APLP2) is a paralogue of APP and is similarly cleaved by secretases to form peptides which may have similar functions to APP cleaved domains, including cell adhesion, migration, cell signaling, and cell cycle regulation. Increased expression of APLP2 has been reported in some tumours. APLP2 was identified in seven cell lines, including TPC-1, BCPAP, CAL62, SW1736, C643, MRO, and WRO. [17]
Clusterin. Clusterin is a glycoprotein that has many biological functions of which are not well understood. It appears to be involved in cell death, tumour progression, tissue differentiation, cell-cell interactions, cell proliferation, lipid transportation, and neurodegenerative disorders. Clusterin was identified in seven cell lines, including TPC-1, CAL62, SW1736, MRO, and WRO. [18]

In summary, by verifying the above protein biomarkers in the sera and tissues of thyroid cancer patients, the feasability of using a secretome approach to identify potential thyroid cancer biomarkers has been illustrated. The findings also reveal the potential for secretome analysis to identify proteins that may help to distinguish between aggressive and non-aggressive carcinomas.

TABLE 1
Novel Thyroid Cancer Markers

| ID | SEQ ID No. | Thyroid Cancer Marker | Accession No. |
|---|---|---|---|
| 1. | SEQ ID-31 | Versican | trm\|Q59FG9 |
| 2. | SEQ ID-6 | Clusterin | spt\|P10909 |
| 3. | SEQ ID-91 | Vacuolar proton pump subunit S1 | spt\|Q15904 |
| 4. | SEQ ID-32 | Gamma-glutamyl hydrolase* | spt\|Q92820 |
| 5. | SEQ ID-66 | Insulin-like growth factor binding | spt\|Q16270 |
| | | protein 7 | |
| 6. | SEQ ID-21 | α-Enolase*** | trm\|Q53FT9 |
| 7. | SEQ ID-82 | Stem cell growth Factor | trm\|Q5U0B9 |
| 8. | SEQ ID-34 | Syndecan-4 | trm\|Q53FN9\|Q53FN9_HUMAN; |
| | | | spt\|P31431\|SDC4_HUMAN |
| 9. | SEQ ID-28 | Fibronectin | trm\|Q6N025 |
| 10. | SEQ ID-70 | Nucleophosmin | spt\|P06748 |
| 11. | SEQ ID-29 | Ubiquitin-A 52-residue ribosomal | trm\|Q3MIH3 |
| | | protein fusion product | |
| 12. | SEQ ID-33 | Lysyl oxidase-like 2* | trm\|Q53HV3\|Q53HV3_HUMAN; |
| | | | spt\|Q9Y4K0\|LOXL2_HUMAN |
| 13. | SEQ ID-18 | Nucleobindin-1 variant | trm\|Q53GX6 |
| 14. | SEQ ID-14 | Calsyntenin-1 | trm\|Q5UE58 |
| 15. | SEQ ID-3 | Prothymosin-α | trm\|Q9NYD3 |
| 16. | SEQ ID-40 | Agrin | spt\|O00468 |
| 17. | SEQ ID-7 | Amyloid-like protein 2 (APLP2)*** | trm\|Q9BT36 |
| 18. | SEQ ID-43 | Beta-2-microglobulin | trm\|Q6IAT8\|Q6IAT8_HUMAN; |
| | | | spt\|P61769\|B2MG_HUMAN |
| 19. | SEQ ID-27 | CD44 antigen | spt\|P16070 |
| 20. | SEQ ID-22 | dystroglycan 1 | trm\|Q969J9 |
| 21. | SEQ ID-17 | Gelsolin** | spt\|P06396 |
| 22. | SEQ ID-35 | hnRNP A2/B1** | spt\|P22626 |
| 23. | SEQ ID-8 | Nucleolin | spt\|P19338 |
| 24. | SEQ ID-11 | SET protein | trm\|Q6FHZ5 |
| 25. | SEQ ID-5 | Biotinidase* | spt\|P43251 |
| 26. | SEQ ID-16 | Nidogen-1* | spt\|P14543 |
| 27. | SEQ ID-15 | Dickkopf-related protein 3 | trm\|Q6PQ81 |
| 28. | SEQ ID-30 | Basement membrane specific | spt\|P98160 |
| | | heparin sulfate core protein** | |
| 29. | SEQ ID-45 | Cadherin 2*** | spt\|P19022 |
| 30. | SEQ ID-62 | Granulins (proepithelin)*** | spt\|P28799 |
| 31. | SEQ ID-2 | Activated Leukocyte Cell | trm\|Q1HGM9 |
| | | Adhesion Molecule (ALCAM) | |
| 32. | SEQ ID-48 | Cathepsin Z*** | trm\|Q5U000 |
| 33. | SEQ ID-64 | Hypothetical protein (belongs to | trm\|Q8WVW5 |
| | | the actin family)*** | |
| 34. | SEQ ID-73 | Peptidylproylisomerase A | trm\|Q3KQW3 |
| 35. | SEQ ID-65 | Insulin-like growth factor binding | spt\|P24592 |
| | | protein 6*** | |
| 36. | SEQ ID-1 | AXL receptor tyrosine kinase*** | trm\|Q8N5L2\|Q8N5L2_HUMAN |
| 37. | SEQ ID-38 | 14-3-3 protein epsilon (14-3-3E) | spt\|P62258 |
| 38. | SEQ ID-10 | 14-3-3 protein zeta/delta (Protein | spt\|P63104 |
| | | kinase C inhibitor protein 1)*** | |
| 39. | SEQ ID-39 | 60S acidic ribosomal protein P2 | spt\|P05387 |
| | | (NY-REN-44 antigen)*** | |
| 40. | SEQ ID-41 | Alpha-actinin-1*** | spt\|P12814 |
| 41. | SEQ ID-42 | Alpha-actinin-4 | spt\|O43707 |
| 42. | SEQ ID-44 | C4B1 (Complement component | trm\|Q6U2E9 |
| | | C4B)*** | |
| 43. | SEQ ID-26 | Calmodulin (CaM)*** | trm\|Q9BRL5\|Q9BRL5_HUMAN; |
| | | | spt\|P62158\|CALM_HUMAN |
| 44. | SEQ ID-46 | Calreticulin (CRP55) | trm\|Q53G71 |
| 45. | SEQ ID-47 | Cathepsin C*** | trm\|Q8WY99 |
| 46. | SEQ ID-49 | CDNA FLJ45706 fis, clone | trm\|Q6ZS99 |
| | | FEBRA2028457, highly similar to | |
| | | Nucleolin*** | |
| 47. | SEQ ID-50 | Chaperonin 10-related protein*** | trm\|Q9UNM1 |
| 48. | SEQ ID-51 | Cofilin-129 | spt\|P23528 |
| 49. | SEQ ID-52 | Collagen alpha-1 (V) chain*** | spt\|P20908 |
| 51. | SEQ ID-54 | Collagen alpha-1 (XII) chain*** | spt\|Q99715 |
| 52. | SEQ ID-55 | Collagen, type I, alpha 2*** | trm\|Q7Z5S6 |
| 53. | SEQ ID-56 | Colony stimulating factor 1 | trm\|Q5VVF4 |
| | | (Macrophage)*** | |
| 54. | SEQ ID-57 | EGF-containing fibulin-like | spt\|Q12805 |
| | | extracellular matrix protein 1*** | |
| 55. | SEQ ID-58 | Filamin A*** | trm\|Q60FE6 |
| 56. | SEQ ID-59 | Follistatin-related protein 1*** | spt\|Q12841 |
| 57. | SEQ ID-60 | Fructose-bisphosphate | trm\|Q6FI10 |
| | | aldolase*** | |
| 58. | SEQ ID-63 | Heat shock protein (HSP 90-alpha | trm\|Q5CAQ7 |
| | | 2)*** | |
| 59. | SEQ ID-61 | Glucose-6-phosphate isomerase | spt\|P06744 |
| 60. | SEQ ID-13 | HNRPK protein (Heterogeneous | trm\|Q5T6W2 |
| | | nuclear ribonucleoprotein K)*** | |
| 61. | SEQ ID-80 | Secretogranin 2 | trm\|Q53T11 |
| 62. | SEQ ID-67 | L-lactate dehydrogenase A chain | spt\|P00338 |
| 63. | SEQ ID-68 | Matrix metalloproteinase 1 (MMP-1) | trm\|Q5TZP0 |
| 64. | SEQ ID-69 | Matrix metalloproteinase 1 | trm\|Q53G75 |
| | | preprotein variant | |
| 65. | SEQ ID-37 | Niemann-Pick disease, type C2 | trm\|Q53HV6 |
| | | variant*** | |
| 66. | SEQ ID-71 | Nucleoside diphosphate kinase | trm\|Q32Q12 |
| | | (NME1-NME2)*** | |
| 67. | SEQ ID-72 | OAF homolog*** | trm\|Q86UD1 |
| 68. | SEQ ID-74 | Phosphoglycerate kinase*** | trm\|Q5J7W1 |
| 69. | SEQ ID-75 | PKM2 protein*** | trm\|Q8WUW7 |
| 70. | SEQ ID-85 | Tissue-type plasminogen activator | trm\|Q6PJA5 |
| | | (PLAT protein)*** | |
| 71. | SEQ ID-76 | Protein CutA*** | spt\|O60888 |
| 72. | SEQ ID-77 | Protein FAM3C*** | spt\|Q92520 |
| 73. | SEQ ID-78 | Protein S100-A9*** | spt\|P06702 |
| 74. | SEQ ID-12 | Pyruvate kinase isozymes | trm\|Q53GK4 |
| | | M1/M2*** | |
| 75. | SEQ ID-79 | Ribosomal protein S27a*** | spt\|P62979 |
| 76. | SEQ ID-81 | SPARC (Secreted protein acidic | spt\|P09486 |
| | | and rich in cysteine) (Osteonectin) | |
| 77. | SEQ ID-83 | Sulfhydryl oxidase 1 (Quiescin | spt\|O00391 |
| | | Q6) (hQSOX)*** | |
| 78. | SEQ ID-84 | Thrombospondin 2*** | trm\|Q5RI52 |
| 79. | SEQ ID-86 | Transforming growth factor, beta- | trm\|Q53GU8 |
| | | induced, 68 kDa variant | |
| 80. | SEQ ID-87 | Transketolase (TK)*** | trm\|Q53EM5 |
| 81. | SEQ ID-88 | Translation elongation factor 1 | trm\|Q96RE1 |
| | | alpha 1-like 14*** | |
| 82. | SEQ ID-89 | Triosephosphate isomerase | trm\|Q6FHP9 |
| 83. | SEQ ID-90 | UV excision repair protein RAD23 | spt\|P54727 |
| | | homolog B*** | |
| 84. | SEQ ID-19 | Cysteine rich angiogenic inducer, | trm\|Q53FA4\|Q53FA4_HUMAN; |
| | | 61 variant (CYR61) | spt\|O00622\|CYR61_HUMAN |
| 85. | SEQ ID-23 | Melanoma associated antigen | rm\|Q92626\|Q92626_HUMAN |
| 86. | SEQ ID-24 | Osteopontin | trm\|Q0JV14\|Q0JV14_HUMAN; |
| | | | spt\|P10451\|OSTP_HUMAN |
| 87. | SEQ ID-25 | Plasminogen activator, urokinase | trm\|Q5SWW9\|Q5SWW9_HUMAN; |
| | | activator | trm\|Q5SWW8\|Q5SWW8_HUMAN; |
| | | | trm\|Q5PY49\|Q5PY49_HUMAN; |
| | | | trm\|Q53XS3\|Q53XS3_HUMAN; |
| | | | spt\|P00749\|UROK_HUMAN |
| 88. | SEQ ID-36 | MCFD2*** | trm\|Q68D61 |

*Novel Papillary Cancer Markers (TPC-1/BCPAP);
**Novel Follicular Cancer Markers (MRO/WRO);
***Novel Aggressive/Metastatic Thyroid Cancer Markers (CAL62, SW1736, C643)

TABLE 2
Known Thyroid Cancer Markers

| S. No | SEQ ID# | Protein | Protein ID |
|---|---|---|---|
| 1. | SEQ ID 4 | Galectin-3 | spt\|Q08380 |
| 2. | SEQ ID 92 | Serum thyroglobulin | spt\|p01266 |
| 3. | SEQ ID 93 | BRAF mutation | spt\|p15056 |
| 4. | SEQ ID 20 | E-Cadherin | spt\|P12830 |
| 5. | SEQ ID 94 | Vimentin | trm\|Q5JVT0 |
| 6. | SEQ ID 95 | Galectin-1 | spt\|p09382 |
| 7. | SEQ ID 9 | Amyloid precursor | AAB19991 |
| | | protein (APP) | |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

### CITATIONS FOR PUBLICATIONS REFERRED TO IN THE SPECIFICATION

1. Damante, G. S., A.; Tell, G.; Thyroid Tumors: novel insights from proteomics studies. Expert Rev Proteomics 2009, 6 (4), 363-376.
2. Carpi, A.; Di Coscio, G.; Iervasi, G.; Antonelli, A.; Mechanick, J.; Sciacchitano, S.; Nicolini, A., Thyroid fine needle aspiration: how to improve clinicians' confidence and performance with the technique. Cancer Lett 2008, 264 (2), 163-71.
3. Nikiforova, M. N.; Nikiforov, Y. E., Molecular diagnostics and predictors in thyroid cancer. Thyroid 2009, 19 (12), 1351-61.
4. Li, H. D., L. V.; Ghanny, S.; Li, W.; Romaschin, A. D.; Colgan, T. J.; Siu, K. W., Identification of candidate biomarker proteins released by human endometrial and cervical cancer cells using two-dimensional liquid chromatography/tandem mass spectrometry. J. Proteome Res. 2007, 7, 2615-2622.
5. (a) DeSouza, L.; Diehl, G.; Rodrigues, M. J.; Guo, J.; Romaschin, A. D.; Colgan, T. J.; Siu, K. W., Search for cancer markers from endometrial tissues using differentially labeled tags iTRAQ and cICAT with multidimensional liquid chromatography and tandem mass spectrometry. J Proteome Res 2005, 4 (2), 377-86; (b) DeSouza, L. V.; Grigull, J.; Ghanny, S.; Dube, V.; Romaschin, A. D.; Colgan, T. J.; Siu, K. W., Endometrial carcinoma biomarker discovery and verification using differentially tagged clinical samples with multidimensional liquid chromatography and tandem mass spectrometry. Mol Cell Proteomics 2007, 6 (7), 1170-82.
6. (a) Soundararajan, S.; Wang, L.; Sridharan, V.; Chen, W.; Courtenay-Luck, N.; Jones, D.; Spicer, E. K.; Fernandes, D. J., Plasma membrane nucleolin is a receptor for the anticancer aptamer AS1411 in MV4-11 leukemia cells. Mol Pharmacol 2009, 76 (5), 984-91; (b) Fogal, V.; Sugahara, K. N.; Ruoslahti, E.; Christian, S., Cell surface nucleolin antagonist causes endothelial cell apoptosis and normalization of tumor vasculature. Angiogenesis 2009, 12 (1), 91-100.
7. Destouches, D.; El Khoury, D.; Hamma-Kourbali, Y.; Krust, B.; Albanese, P.; Katsoris, P.; Guichard, G.; Briand, J. P.; Courty, J.; Hovanessian, A. G., Suppression of tumor growth and angiogenesis by a specific antagonist of the cell-surface expressed nucleolin. PLoS One 2008, 3 (6), e2518.
8. Chen, C.-C.; Lau, L. F., Functions and mechanisms of action of CCN matricellular proteins. The International Journal of Biochemistry & Cell Biology 2009, 41 (4), 771-783.
9. Leask, A., A sticky situation: CCN1 promotes both proliferation and apoptosis of cancer cells. Journal of Cell Communication and Signaling.
10. Makrilia, N.; Kollias, A.; Manolopoulos, L.; Syrigos, K., Cell adhesion molecules: role and clinical significance in cancer. Cancer Invest 2009, 27 (10), 1023-37.
11. Letsas, K. P.; Vartholomatos, G.; Tsepi, C.; Tsatsoulis, A.; Frangou-Lazaridis, M., Fine-needle aspiration biopsy-RT-PCR expression analysis of prothymosin alpha and parathymosin in thyroid: novel proliferation markers? Neoplasma 2007, 54 (1), 57-62.
12. Ofori-Acquah S F, King J A. Activated leukocyte cell adhesion molecule: a new paradox in cancer. Transl Res. 2008 March; 151(3):122-8.
13. Swart G W. Activated leukocyte cell adhesion molecule (CD166/ALCAM): Developmental and mechanistic aspects of cell clustering and cell migration. European Journal of Cell Biology 81, 313±321.
14. Hafizi S, Dahlbäck B. Signaling and functional diversity within the Axl subfamily of receptor tyrosine kinases. Cytokine & Growth Factor Reviews 17 (2006) 295-304.
15. Keun-A Chang & Yoo-Hun Suh, Possible roles of amyloid intracellular domain of amyloid. BMB Rep. 2010 October; 43(10):656-63.
16. Chen M, Zhang J, Manley J L. Turning on a fuel switch of cancer: hnRNP proteins regulate alternative splicing of pyruvate kinase mRNA. Cancer Res. 2010 Nov. 15; 70(22):8977-80.
17. Amit Tuli•Mahak Sharma•Xiaojian Wang•Laura C. Simone•Haley L. Capek Steven Cate•William H. Hildebrand•Naava Naslaysky•Steve Caplan•Joyce C. Solheim, Amyloid precursor-like protein 2 association with HLA class I molecules, Cancer Immunol Immunother (2009) 58:1419-1431.
18. Federica Rizzi and Saverio Bettuzzi, The clusterin paradigm in prostate and breast carcinogenesis, Endocr Relat Cancer. 2010 Jan. 29; 17(1):R1-17.

Sequence Listing

```
SEQ ID No. 1
tyrosine-protein kinase receptor UFO (AXL)
trm|Q8N5L2
      1 mawrcprmgr vplawclalc gwacmaprgt qaeespfvgn pgnitgargl tgtlrcqlqv
    61 qgeppevhwl rdgqilelad stqtqvplge deqddwivvs qlritslqls dtgqyqclvf
  121 lghqtfvsqp gyvgleglpy fleepedrtv aantpfnlsc qaggppepvd llwlqdavpl
  181 atapghgpqr slhvpglnkt ssfsceahna kgvttsrtat itvlpqqprn lhlvsrqpte
  241 levawtpgls giyplthctl gavlsddgmg iqagepdppe epltsqasvp phqlrlgslh
  301 phtpyhirva ctssqgpssw thwlpvetpe gvplgppeni satrngsgaf vhwqeprapl
  361 qgtllgyrla yqgqdtpevl mdiglrqevt lelqgdgsvs nltvcvaayt aagdgpwslp
  421 vpleawrpgq aqpvhqlvke pstpafswpw wyvllgavva aacvlilalf lvhrrkketr
  481 ygevfeptve rgelvvryrv rksysrrtte atlnslgise elkeklrdvm vdrhkvalgk
  541 tlgegefgav megqlnqdds ilkvavktmk iaictrsele dflseavcmk efdhpnvmrl
  601 igvcfqgser esfpapvvil pfmkhgdlhs fllysrlgdq pvylptqmlv kfmadiasgm
  661 eylstkrfih rdlaarncml nenmsvcvad fglskkiyng dyyrqgriak mpvkwiaies
  721 ladrvytsks dvwsfgvtmw eiatrgqtpy pgvenseiyd ylrrgnrlkq padcldglya
  781 lmsrcwelnp qdrpsftelr edlentlkal ppaqepdeil yvnmdegggy peppgaagga
  841 dpptqpdpkd scscltaaev hpagryvlcp sttpspaqpa drgspaapgq edga
SEQ ID No. 2
activated leukocyte cell adhesion molecule (ALCAM)/CD166
trm|Q1HGM9
      1 meskgasscr llfcllisat vfrpglgwyt vnsaygdtii ipcrldvpqn lmfgkwkyek
    61 pdgspvfiaf rsstkksvqy ddvpeykdrl nlsenytlsi snarisdekr fvcmlvtedn
  121 vfeaptivkv fkqpskpeiv skalfleteq lkklgdcise dsypdgnitw yrngkvlhpl
  181 egavviifkk emdpvtqlyt mtstleyktt kadiqmpftc svtyygpsgq ktihsegavf
  241 diyypteqvt iqvlppknai kegdnitlkc lgngnpppee flfylpgqpe girssntytl
  301 tdvrrnatgd ykcslidkks miastaitvh yldlslnpsg evtrqigdal pvsctisasr
  361 natvvwmkdn irlrsspsfs slhyqdagny vcetalqeve glkkresltl ivegkpqikm
  421 tkktdpsgls ktiichvegf pkpaiqwtit gsgsvinqte espyingryy skiiispeen
  481 vtltctaenq lertvnslnv saisipehde adeisdenre kvndqakliv givvglllaa
  541 lvagvvywly mkksktaskh vnkdlgnmee nkkleennhk tea
SEQ ID No. 3
prothymosin alpha (PTMA)
trm|Q9NYD3
      1 msdaavdtss eittedlkek kevveeaeng rdapahgnan eengepeadn evdeeeeegg
    61 eeegdgeeed gdedegaesa tgkraaedde dddvdtqkqk tdedd
SEQ ID No. 4
galectin-3
spt|Q08380
      1 mrflaatfll lalstaaqae pvqfrdcgsv dgvikevnvs pcptqpcqls kgqsysvnvt
    61 ftsnvqskss kavvhgilmg vpvpfpipep dgcksgincp iqkdktysyl nklpvkseyp
  121 siklvvewql qddknqslfc weipvgivsh l
SEQ ID No. 5
Biotinidase
      1 mddredlvyq aklaeqaery demvesmkkv agmdveltve ernllsvayk nvigarrasw
    61 riissieqke enkggedklk mireyrqmve telkliccdi ldvldkhlip aantgeskvf
  121 yykmkgdyhr ylaefatgnd rkeaaenslv aykaasdiam telppthpir lglalnfsvf
  181 yyeilnspdr acrlakaafd daiaeldtls eesykdstli mqllrdnltl wtsdmqgdge
  241 eqnkealqdv edenq
SEQ ID No. 6
Clusterin
spt|P10909
      1 mmktlllfvg llltwesgqv lgdqtvsdne lqemsnqgsk yvnkeiqnav ngvkqiktli
    61 ektneerktl lsnleeakkk kedalnetre setklkelpg vcnetmmalw eeckpclkqt
  121 cmkfyarvcr sgsglvgrql eeflnqsspf yfwmngdrid sllendrqqt hmldvmqdhf
  181 srassiidel fqdrfftrep qdtyhylpfs lphrrphfff pksrivrslm pfspyepinf
  241 hamfqpflem iheaqqamdi hfhspafqhp ptefiregdd drtvcreirh nstgclrmkd
  301 qcdkcreils vdcstnnpsq aklrreldes lqvaerltrk ynellksyqw kmlntsslle
  361 qlneqfnwvs rlanltqged qyylrvttva shtsdsdvps gvtevvvklf dsdpitvtvp
  421 vevsrknpkf metvaekalq eyrkkhree
SEQ ID No. 7
APLP2
trm|Q9BT36
      1 maatgtaaaa atgrllllll vgltapalal agyiealaan agtgfavaep qiamfcgkln
    61 mhvniqtgkw epdptgtksc fetkeevlqy cqemypelqi tnvmeanqrv sidnwcrrdk
  121 kqcksrfvtp fkclvpptpl ptndvdvyfe tsaddnehar fqkakeqlei rhrnrmdrvk
  181 keweeaelqa knlpkaerqt liqhfqamvk alekeaasek qqlvethlar veamlndrrr
  241 malenylaal qsdpprphri lqalrryvra enkdrlhtir hyqhvlavdp ekaaqmksqv
  301 mthlhvieer rnqtlsllyk vpyvaqeiqe eidellqeqr admdqftasi setpvdvrvs
  361 seeseeippf hpfhpfpalp enegsgvgeq dggligaeek vinsknkvde nmvidetldv
  421 kemifnaerv ggleeeresv gplredfsls ssaligllvi avaiatvivi slvmlrkrqy
  481 gtishgivev dpmltpeerh lnkmqnhgye nptykyleqm qi
SEQ ID No.8
nucleolin
spt|P19338
      1 mvklakagkn qgdpkkmapp pkeveedsed eemsedeedd ssgeevvipq kkgkkaaats
    61 akkvvvsptk kvavatpakk aavtpgkkaa atpakktvtp akavttpgkk gatpgkalva
  121 tpgkkgaaip akgakngkna kkedsdeeed ddseedeedd ededededei epaamkaaaa
  181 apasededde ddeddedddd deeddseeea mettpakgkk aakvvpvkak nvaededeee
  241 ddededdddd eddedddded deeeeeeeee epvkeapgkr kkemakqkaa peakkqkveg
  301 tepttafnlf vgnlnfnksa pelktgisdv fakndlavvd vrigmtrkfg yvdfesaedl
  361 ekaleltglk vfgneiklek pkgkdskker dartllaknl pykvtqdelk evfedaaeir
  421 lvskdgkskg iayiefktea daektfeekq gteidgrsis lyytgekgqn qdyrggknst
  481 wsgesktlvl snlsysatee tlqevfekat fikvpqnqng kskgyafief asfedakeal
  541 nscnkreieg rairlelqgp rgspnarsqp sktlfvkgls edtteetlke sfdgsvrari
  601 vtdretgssk gfgfvdfnse edakaakeam edgeidgnkv tldwakpkge ggfggrgggr
  661 ggfggrgggr ggrggfggrg rggfggrggf rggrggggdh kpqgkktkfe
SEQ ID No. 9
amyloid precursor protein (APP)
Acession No. AAB19991
      1 vffaedvgsn kgaiiglmvg gvviatvifi tlvmlkkkqy tsihhgvve
SEQ ID No. 10
14-3-3 Zeta
sp|P63104
      1 mdknelvqka klaeqaeryd dmaacmksvt eqgaelsnee rnllsvaykn vvgarrsswr
    61 vvssieqkte gaekkqqmar eyrekietel rdicndvlsl lekflipnas qaeskvfylk
  121 mkgdyyryla evaagddkkg ivdqsqqayq eafeiskkem qpthpirlgl alnfsvfyye
  181 ilnspekacs laktafdeai aeldtlsees ykdstlimql lrdnltlwts dtqgdeaeag
  241 eggen
SEQ ID No. 11
SET
trm|Q6FHZ5
      1 msaqaakvsk kelnsnhdga detsekeqqe aiehidevqn eidrlneqas eeilkveqky
    61 nklrqpffqk rseliakipn fwvttfvnhp qvsallgeed eealhyltrv evtefediks
  121 gyridfyfde npyfenkvls kefhlnesgd pssksteikw ksgkdltkrs sqtqnkasrk
  181 rqheepesff twftdhsdag adelgevikd diwpnplqyy lvpdmddeeg egeeddddde
  241 eeegledide egdedegeed edddegeege edegedd
SEQ ID No. 12
PKM2
trm|Q53GK4
      1 mskphseagt afiqtqqlha amadtflehm crldidsppi tarntgiict igpasrsvet
    61 lkemiksgmn varlnfshgt heyhaetikn vrtatesfas dpilyrpvav aldtkgpeir
  121 tglikgsgta evelkkgatl kitldnayme kcdenilwld yknickvvev gskiyvddgl
  181 islqvkrkga dflvteveng gslgskkgvn lpgaavdlpa vsekdiqdlk fgveqdvdmv
  241 fasfirkasd vrevrkvlge kgknikiisk ienhegvrrf deileasdgi mvargdlgie
  301 ipaekvflaq kmmigrcnra gkpvicatqm lesmikkprp traegsdvan avldgadcim
  361 lsgetakgdy pleavrmqhl iareaeaaiy hlqlfeelrr lapitsdpte atavgaveas
  421 fkccsgaiiv ltksgrsahq varyrprapi iavtrnpqta rqahlyrgif pvlckdpvqe
  481 awaedvdlrv nfamnvgkar gffkkgdvvi vltgwrpgsg ftntmrvvpv p
SEQ ID No. 13
hnRNPK
trm|Q5T6W2
      1 metempeetf pntetngefg krpaedmeee qafkrsrntd emvelrillq sknagavigk
    61 ggknikalrt dynasvsvpd ssgperilsi sadietigei lkkiiptlee yqhykgsdfd
  121 celrllihqs laggiigvkg akikelrent qttiklfqec cphstdrvvl iggkpdrvve
  181 cikiildlis espikgraqp ydpnfydety dyggftmmfd drrgrpvgfp mrgrggfdrm
  241 ppgrggrpmp psrrdyddms prrgpppppp grggrggsra rnlplppppp prggdlmayd
  301 rrgrpgdryd gmvgfsadet wdsaidtwsp sewqmayepq ggsgydysya ggrgsygdlg
  361 gpiittqvti pkdlagsii
SEQ ID No. 14
calsyntenin-1
trm|Q5UE58
      1 mlrrpapala paarlllagl lcgggvwaar vnkhkpwlep tyhgivtend ntvlldppli
    61 aldkdaplrf ageicgfkih gqnvpfdavv vdkstgegvi rskekldcel qkdysftiqa
  121 ydcgkgpdgt nvkkshkatv hiqvndvney apvfkeksyk atviegkqyd silrveavda
  181 dcspqfsqic syeiitpdvp ftvdkdgyik nteklnygke hqykltvtay dcgkkrated
  241 vlvkisikpt ctpgwqgwnn rieyepgtga lavfpnihle tcdepvasvq atveletshi
  301 gkgcdrdtys ekslhrlcga aagtaellps psgslnwtmg lptdnghdsd qvfefngtqa
  361 vripdgvvsv spkepftisv wmrhgpfgrk ketilcssdk tdmnrhhysl yvhgcrlifl
  421 frqdpseekk yrpaefhwkl nqvcdeewhh yvlnvefpsv tlyvdgtshe pfsvtedypl
  481 hpskietqlv vgacwqefsg vendnetepv tvasaggdlh mtqffrgnla gltlrsgkla
  541 dkkvidclyt ckegldlqvl edsgrgvqiq ahpsqlvltl egedlgeldk amqhisylns
  601 rqfptpgirr lkitstikcf neatcisvpp vdgyvmvlqp eepkislsgv hhfaraasef
  661 essegvflfp elriistitr evepegdgae dptvqeslvs eeivhdldtc evtvegeeln
  721 heqeslevdm arlqqkgiev ssselgmtft gvdtmasyee vlhllryrnw harslldrkf
  781 klicselngr yisnefkvev nvihtanpme hanhmaaqpq fvhpehrsfv dlsghnlanp
  841 hpfavvpsta tvvivvcvsf lvfmiilgvf riraahrrtm rdqdtgkene mdwddsalti
  901 tvnpmetyed qhsseeeeee eeeeesedge eedditsaes esseeeegeq gdpqnatrqq
  961 qlewddstls y
SEQ ID No. 15
Dickkopf-related protein 3 (DKK-3)
trm|Q6PQ81
      1 mqrlgatllc lllaaavpta papaptatsa pvkpgpalsy pqeeatlnem freveelved
    61 tqhklrsave emeaeeaaak assevnlanl ppsyhnetnt dtkvgnntih vhreihkitn
  121 nqarqmvfse tvitsvgdee grrsheciid edcgpsmycq fasfqytcqp crgqrmlctr
  181 dseccgdqlc vwghctkmat rgsngticdn qrdcqpglcc afqrgllfpv ciplpvegel
  241 chdpasrlld litwelepdg aldrcpcasg llcqphshsl vyvckptfvg srdqdgeill
  301 prevpdeyev gsfmeevrqe ledlerslte emalgepaaa aaallggeei
SEQ ID No. 16
nidogen-1
spt|P14543
      1 mlasssrira awtralllpl llagpvgcls rqelfpfgpg qgdleledgd dfvspalels
    61 galrfydrsd idavyvttng iiatseppak eshpglfppt fgavapflad ldttdglgkv
  121 yyredlspsi tqraaecvhr gfpeisfqps savvvtwesv apyqgpsrdp dqkgkrntfq
  181 avlassdsss yaiflypedg lqfhttfskk ennqvpavva fsqgsvgflw ksngaynifa
  241 ndresvenla kssnsgqqgv wvfeigspat tngvvpadvi lgtedgaeyd dededydlat
  301 trlgledvgt tpfsykalrr ggadtysvps vlsprraate rplgpptert rsfqlavetf
  361 hqqhpqvidv deveetgvvf syntdsrqtc annrhqcsvh aecrdyatgf ccscvagytg
  421 ngrqcvaegs pqrvngkvkg rifvgssqvp ivfentdlhs yvvmnhgrsy taistipetv
  481 gysllplapv ggiigwmfav eqdgfkngfs itggeftrqa evtfvghpgn lvikqrfsgi
  541 dehghltidt elegrvpqip fgssvhiepy telyhystsv itssstreyt vteperdgas
  601 psriytyqwr qtitfqecvh ddsrpalpst qqlsvdsvfv lynqeekilr yalsnsigpv
  661 regspdalqn pcyigthgcd tnaacrpgpr tqftcecsig frgdgrtcyd idecseqpsv
  721 cgshticnnh pgtfrcecve gyqfsdegtc vavvdqrpin ycetglhncd ipqraqciyt
  781 ggssytcscl pgfsgdgqac qdvdecqpsr chpdafcynt pgsftcqckp gyqgdgfrcv
  841 pgevektrcq herehilgaa gatdpqrpip pglfvpecda hghyaptqch gstgycwcvd
  901 rdgrevegtr trpgmtppcl stvappihqg pavptavipl ppgthllfaq tgkierlple
  961 gntmrkteak aflhvpakvi iglafdcvdk mvywtditep sigraslhgg epttiirqdl
1021 gspegiavdh lgrnifwtds nldrievakl dgtqrrvlfe tdlvnprgiv tdsvrgnlyw
1081 tdwnrdnpki etsymdgtnr rilvqddlgl pngltfdafs sqlcwvdagt nraeclnpsq
1141 psrrkalegl qypfavtsyg knlyftdwkm nsvvaldlai sketdafqph kqtrlygitt
1201 alsqcpqghn ycsvnnggct hlclatpgsr tcrcpdntlg vdcieqk
SEQ ID NO. 17
Gelsolin
spt|P06396
      1 maphrpapal lcalslalca lslpvraata srgasqagap qgrvpearpn smvvehpefl
    61 kagkepglqi wrvekfdlvp vptnlygdff tgdayvilkt vqlrngnlqy dlhywlgnec
  121 sqdesgaaai ftvqlddyln gravqhrevq gfesatflgy fksglkykkg gvasgfkhvv
  181 pnevvvqrlf qvkgrrvvra tevpvswesf nngdcfildl gnnihqwcgs nsnryerlka
  241 tqvskgirdn ersgrarvhv seegtepeam lqvlgpkpal pagtedtake daanrklakl
  301 ykvsngagtm syslvadenp faqgalksed cfildhgkdg kifvwkgkqa nteerkaalk
  361 tasdfitkmd ypkqtqvsvl peggetplfk qffknwrdpd qtdglglsyl sshianverv
  421 pfdaatlhts tamaaqhgmd ddgtgqkqiw riegsnkvpv dpatygqfyg gdsyiilyny
  481 rhggrqgqii ynwqgaqstq devaasailt aqldeelggt pvqsrvvqgk epahlmslfg
  541 gkpmiiykgg tsreggqtap astrlfqvra nsagatrave vlpkagalns ndafvlktps
  601 aaylwvgtga seaektgaqe llrvlraqpv qvaegsepdg fwealggkaa yrtsprlkdk
  661 kmdahpprlf acsnkigrfv ieevpgelmq edlatddvml ldtwdqvfvw vgkdsqeeek
  721 tealtsakry ietdpanrdr rtpitvvkqg feppsfvgwf lgwdddywsv dpldramael
  781 aa
SEQ ID No. 18
Nucleobindin
trm|Q53GX6
      1 mppsgprgtl lllpllllll lravlavple rgapnkeetp atespdtgly yhrylqevid
    61 vletdghfre klqaanaedi ksgklsreld fvshhvrtrl delkrqevsr lrmllkakmd
  121 aeqdpnvqvd hlnllkqfeh ldpqnqhtfe ardlelliqt atrdlaqyda ahheefkrye
  181 mlkeherrry leslgeeqrk eaerkleeqq rrhrehpkvn vpgsgaqlke vweeldgldp
  241 nrfnpktffi lhdinsdgvl deqelealft kelekvydpk needdmreme eerlrmrehv
  301 mknvdtnqdr lvtleeflas tqrkefgdtg egwetvemhp ayteeelrrf eeelaareae
  361 lnakaqrlsq etealgrsqg rleaqkrelq gavlhmeqrk qqqqqqqghk apaahpegql
  421 kfhpdtddvp vpapagdgke vdtsekklle rlpevevpqh l
SEQ ID No. 19
CYR61
trm|Q53FA4
      1 mssriarala lvvtllhltr lalstcpaac hcpleapkca pgvglvrdgc gcckvcakql
    61 nedcsktqpc dhtkglecnf gasstalkgi craqsegrpc eynsriyqng esfqpsckhq
  121 ctcidgavgc iplcpqelsl pnlgcpnprl vkvtgqccee wvcdedsikd pmedqdgllg
  181 kelgfdasev eltrnnelia vgkgsslkrl pvfgmepril ynplqgqkci vqttswsqcs
  241 ktcgtgistr vtndnpecrl vketricevr pcgqpvyssl kkgkkcsktk kspepvrfty
  301 agclsvkkyr pkycgsrvdg rcctpqltrt vkmrfrcedg etfsknvmmi qsckcnyncp
  361 haneaafpfy rlfndihkfr g
SEQ ID No. 20
E-cadherin
sp|P12830
      1 mgpwsrslsa lllllqvssw lcqepepchp gfdaesytft vprrhlergr vlgrvnfedc
    61 tgrqrtayfs ldtrfkvgtd gvitvkrplr fhnpqihflv yawdstyrkf stkvtlntvg
  121 hhhrppphqa sysgiqaell tfpnsspglr rqkrdwvipp iscpenekgp fpknlvqiks
  181 nkdkegkvfy sitgqgadtp pvgvfiiere tgwlkvtepl dreriatytl fshavssngn
  241 avedpmeili tvtdqndnkp eftgevfkgs vmegalpgts vmevtatdad ddvntynaai
  301 aytilsqdpe lpdknmftin rntgvisvvt tgldresfpt ytlvvqaadl qgeglsttat
  361 avitvtdtnd nppifnptty kgqvpenean vvittlkvtd adapntpawe avytilnddg
  421 gqfvvttnpv nndgilktak gldfeakqqy ilhvavtnvv pfevslttst atvtvdvldv
  481 neapifvppe krvevsedfg vgqeitsyta qepdtfmeqk ityriwrdta nwleinpdtg
  541 aistraeldr edfehvknst ytaliiatdn gspvatgtgt lllilsdvnd napipeprti
  601 ffcernpkpq viniidadlp pntspftael thgasanwti qyndptqesi ilkpkmalev
  661 gdykinlklm dnqnkdqvtt levsvcdceg aagvcrkaqp veaglqipai lgilggilal
  721 lililllllf lrrravvkep llppeddtrd nvyyydeegg geedqdfdls qlhrgldarp
  781 evtrndvapt lmsvprylpr panpdeignf idenlkaadt dptappydsl lvfdyegsgs
  841 eaaslsslns sesdkdqdyd ylnewgnrfk kladmyggge dd
SEQ ID No. 21
α-Enolase
trm|Q53FT9
      1 msilkihare ifdsrgnptv evdlftskgl fraavpsgas tgiyealelr dndktrymgk
    61 gvskavehin ktiapalvsk klnvteqeki dklmiemdgt enkskfgana ilgvslavck
  121 agavekgvpl yrhiadlagn sevilpvpaf nvinggshag nklamqefmi lpvgaanfre
  181 amrigaevyh nlknvikery gkdatnvgde ggfapnilen keglellkta igkagytdkv
  241 vigmdvaase ffrsgkydld fkspddpsry ispdgladly ksfikdypvv siedpfdqdd
  301 wgawqkftas agiqvvgddl tvtnpkriak avnekscncl llkvnqigsv teslqackla
  361 qangwgvmvs hrsgetedtf iadlvvglct gqiktgapcr serlakynql lrieeelgsk
  421 akfagrnfrn plak
SEQ ID No. 22
dystroglycan 1
trm|Q969J9
      1 mrmsvglsll lplwgrtfll llsvvmaqsh wpsepseavr dwenqleasm hsvlsdlhea
    61 vptvvgipdg tavvgrsfrv tiptdliass gdiikvsaag kealpswlhw dsqshtlegl
  121 pldtdkgvhy isvsatrlga ngshipqtss vfsievyped hselqsvrta spdpgevvss
  181 acaadepvtv ltvildadlt kmtpkqridl lhrmrsfsev elhnmklvpv vnnrlfdmsa
  241 fmagpgnakk vvengallsw klgcslnqns vpdihgveap aregamsaql gypvvgwhia
  301 nkkpplpkrv rrqihatptp vtaigpptta iqeppsrivp tptspaiapp tetmappvrd
  361 pvpgkptvti rtrgaiiqtp tlgpiqptrv seagttvpgq irptmtipgy veptavatpp
  421 ttttkkprvs tpkpatpstd stttttrrpt kkprtprpvp rvttkvsitr letaspptri
  481 rtttsgvprg gepnqrpelk nhidrvdawv gtyfevkips dtfydhedtt tdklkltlkl
  541 reqqlvgeks wvqfnsnsql myglpdsshv gkheyfmhat dkgglsavda feihvhrrpq
  601 gdraparfka kfvgdpalvl ndihkkialv kklafafgdr ncstitiqni trgsivvewt
  661 nntlplepcp keqiaglsrr iaeddgkprp afsnalepdf katsitvtgs gscrhlqfip
  721 vvpprrvpse apptevpdrd peksseddvy lhtvipavvv aailliagii amicyrkkrk
  781 gkltledqat fikkgvpiif adelddskpp psssmplilq eekaplpppe ypngsvpett
  841 pinqdtmgey tplrdedpna ppyqppppft apmegkgsrp knmtpyrspp pyvpp
SEQ ID No. 23
melanoma-associated antigen
trm|Q92626
      1 makrsrgpgr rcllalvlfc awgtlavvaq kpgagcpsrc lcfrttvrcm hllleavpav
    61 apqtsildlr fnrireiqpg afrrlrnlnt lllnnnqikr ipsgafedle nlkylylykn
  121 eiqsidrqaf kglasleqly lhfnqietld pdsfqhlpkl erlflhnnri thlvpgtfnh
  181 lesmkrlrld sntlhcdcei lwladllkty aesgnaqaaa iceyprriqg rsvatitpee
  241 lncerprits epqdadvtsg ntvyftcrae gnpkpeiiwl rnnnelsmkt dsrlnllddg
  301 tlmiqntqet dqgiyqcmak nvagevktqe vtlryfgspa rptfviqpqn tevlvgesvt
  361 lecsatghpp priswtrgdr tplpvdprvn itpsgglyiq nvvqgdsgey acsatnnids
  421 vhatafiivq alpqftvtpq drvviegqtv dfqceakgnp ppviawtkgg sqlsvdrrhl
  481 vlssgtlris gvalhdqgqy ecqavniigs qkvvahltvg prvtpvfasi psdttvevga
  541 nvqlpcssqg epepaitwnk dgvqvtesgk fhispegflt indvgpadag ryecvarnti
  601 gsasysmvls vnvpdvsrng dpfvatsive aiatvdrain strthlfdsr prspndllal
  661 fryprdpytv eqarageife rtlqliqehv qhglmvdlng tsyhyndlvs pgylnlianl
  721 sgctahrrvn ncsdmcfhqk yrthdgtcnn lqhpmwgasl taferllksv yengfntprg
  781 inphrlyngh alpmprlvst tligtetvtp deqfthmlmq wgqfldhdld stvvalsqar
  841 fsdgqhcsnv csndppcfsv mippndsrar sgarcmffvr sspvcgsgmt sllmnsvypr
  901 eqinqltsyi dasnvygste hearsirdla shrgllrqgi vqrsgkpllp fatgpptecm
  961 rdenespipc flagdhrane qlgltsmhtl wfrehnriat ellklnphwd gdtiyyetrk
1021 ivgaeiqhit yqhwlpkilg evgmrtlgey hgydpginag ifnafataaf rfghtlvnpl
1081 lyrldenfqp iaqdhlplhk affspfrivn eggidpllrg lfgvagkmrv psqllntelt
1141 erlfsmahtv aldlaainiq rgrdhgippy hdyrvycnls aahtfedlkn eiknpeirek
1201 lkrlygstln idlfpalvve dlvpgsrlgp tlmcllstqf krlrdgdrlw yenpgvfspa
1261 qltqikqtsl arilcdnadn itrvqsdvfr vaefphgygs cdeiprvdlr vwqdccedcr
1321 trgqfnafsy hfrgrrslef syqedkptkk trprkipsvg rqgehlsnst safstrsdas
1381 gtndfrefvl emqktitdlr tqikklesrl sttecvdagg eshanntkwk kdacticeck
1441 dgqvtcfvea cppatcavpv nipgaccpvc lqkraeekp
SEQ ID No. 24
Osteopontin
spt|P10451
      1 mriavicfcl lgitcaipvk qadsgsseek qlynkypdav atwlnpdpsq kqnllapqna
    61 vsseetndfk qetlpsksne shdhmddmdd eddddhvdsq dsidsndsdd vddtddshqs
  121 deshhsdesd elvtdfptdl patevftpvv ptvdtydgrg dsvvyglrsk skkfrrpdiq
  181 ypdatdedit shmeseelng aykaipvaqd lnapsdwdsr gkdsyetsql ddqsaethsh
  241 kqsrlykrka ndesnehsdv idsgelskvs refhshefhs hedmlvvdpk skeedkhlkf
  301 risheldsas sevn
SEQ ID No. 25
plasminogen activator urokinase
spt|P00749
      1 mrallarlll cvlvvsdskg snelhqvpsn cdclnggtcv snkyfsnihw cncpkkfggq
    61 hceidksktc yegnghfyrg kastdtmgrp clpwnsatvl qqtyhahrsd alqlglgkhn
  121 ycrnpdnrrr pwcyvqvglk plvqecmvhd cadgkkpssp peelkfqcgq ktlrprfkii
  181 ggefttienq pwfaaiyrrh rggsvtyvcg gslispcwvi sathcfidyp kkedyivylg
  241 rsrlnsntqg emkfevenli lhkdysadtl ahhndiallk irskegrcaq psrtiqticl
  301 psmyndpqfg tsceitgfgk enstdylype qlkmtvvkli shrecqqphy ygsevttkml
  361 caadpqwktd scqgdsggpl vcslqgrmtl tgivswgrgc alkdkpgvyt rvshflpwir
  421 shtkeengla l
SEQ ID No. 26
calmodulin
spt|P62158
      1 madqlteeqi aefkeafslf dkdgdgtitt kelgtvmrsl gqnpteaelq dminevdadg
    61 ngtidfpefl tmmarkmkdt dseeeireaf rvfdkdgngy isaaelrhvm tnlgekltde
  121 evdemiread idgdgqvnye efvqmmtak
SEQ ID No. 27
CD44 antigen
spt|P16070
      1 mdkfwwhaaw glclvplsla qidlnitcrf agvfhvekng rysisrteaa dlckafnstl
    61 ptmaqmekal sigfetcryg fieghvvipr ihpnsicaan ntgvyiltsn tsqydtycfn
  121 asappeedct svtdlpnafd gpititivnr dgtryvqkge yrtnpediyp snptdddvss
  181 gssserssts ggyifytfst vhpipdedsp witdstdrip attlmstsat atetatkrqe
  241 twdwfswlfl psesknhlht ttqmagtssn tisagwepne enederdrhl sfsgsgiddd
  301 edfisstist tprafdhtkq nqdwtqwnps hsnpevllqt ttrmtdvdrn gttayegnwn
  361 peahpplihh ehheeeetph ststiqatps stteetatqk eqwfgnrwhe gyrqtpkeds
  421 hsttgtaaas ahtshpmqgr ttpspedssw tdffnpishp mgrghqagrr mdmdsshsit
  481 lqptanpntg lvedldrtgp lsmttqqsns qsfstshegl eedkdhptts tltssnrndv
  541 tggrrdpnhs egsttllegy tshyphtkes rtfipvtsak tgsfgvtavt vgdsnsnvnr
  601 slsgdqdtfh psggshtthg sesdghshgs qegganttsg pirtpqipew liilasllal
  661 alilavciav nsrrrcgqkk klvinsgnga vedrkpsgln geasksqemv hlvnkesset
  721 pdqfmtadet rnlqnvdmki gv
SEQ ID No. 28
fibronectin
trm|Q6N025
      1 gprrlcctgg gegtpgasgk rgpaattslv lcipsvpppv pfptlwppps wrrqppggir
    61 rdfsrrlrre anlvatclpv raslphrlnm lrgpgpglll lavlclgtav pstgaskskr
  121 qaqqmvqpqs pvavsgskpg cydngkhyqi nqqwertylg nalvctcygg srgfnceskp
  181 eaeetcfdky tgntyrvgdt yerpkdsmiw dctcigagrg risctianrc heggqsykig
  241 dtwrrphetg gymlecvclg ngkgewtckp iaekcfdhaa gtsyvvgetw ekpycgwmmv
  301 dctclgegsg ritctsrnrc ndqdtrtsyr igdtwskkdn rgnllqcict gngrgewkce
  361 rhtsvqttss gsgpftdara avyqpqphpq pppyghcvtd sgvvysvgmq wlktqgnkqm
  421 lctclgngvs cqetavtqty ggnsngepcv lpftyngrtf yscttegrqd ghlwcsttsn
  481 yeqdqkysfc tdhtvlvqtr ggnsngalch fpflynnhny tdctsegrrd nmkwcgttqn
  541 ydadqkfgfc pmaaheeict tnegvmyrig dqwdkqhdmg hmmrctcvgn grgewtciay
  601 sqlrdqcivd ditynvndtf hkrheeghml nctcfgqgrg rwkcdpvdqc qdsetgtfyq
  661 igdswekyvh gvryqcycyg rgigewhcqp lqtypsssgp vevfitetps qpnshpiqwn
  721 apqpshisky ilrwrpknsv grwkeatipg hlnsytikgl kpgvvyegql isiqqyghqe
  781 vtrfdfttts tstpvtsntv tgettpfspl vatsesvtei tassfvvswv sasdtvsgfr
  841 veyelseegd epqyldlpst atsvnipdll pgrkyivnvy qisedgeqsl ilstsqttap
  901 dappdptvdq vddtsivvrw srpqapitgy rivyspsveg sstelnlpet ansvtlsdlq
  961 pgvqynitiy aveenqestp vviqqettgt prsdtvpspr dlqfvevtdv kvtimwtppe
1021 savtgyrvdv ipvnlpgehg qrlpisrntf aevtglspgv tyyfkvfavs hgreskplta
1081 qqttkldapt nlqfvnetds tvlvrwtppr agitgyrltv gltrrgqprq ynvgpsvsky
1141 plrnlqpase ytvslvaikg nqespkatgv fttlqpgssi ppyntevtet tivitwtpap
1201 rigfklgvrp sqggeaprev tsdsgsivvs gltpgveyvy tiqvlrdgqe rdapivnkvv
1261 tplspptnlh leanpdtgvl tvswersttp ditgyrittt ptngqqgnsl eevvhadqss
1321 ctfdnlspgl eynvsvytvk ddkesvpisd tiipavpppt dlrftnigpd tmrvtwappp
1381 sidltnflvr yspvkneedv aelsispsdn avvltnllpg teyvvsvssv yeqhestplr
1441 grqktgldsp tgidfsdita nsftvhwiap ratitgyrir hhpehfsgrp redrvphsrn
1501 sitltnltpg teyvvsival ngreesplli gqqstvsdvp rdlevvaatp tslliswdap
1561 avtvryyrit ygetggnspv qeftvpgsks tatisglkpg vdytitvyav tgrgdspass
1621 kpisinyrte idkpsqmqvt dvqdnsisvk wlpssspvtg yrvtttpkng pgptktktag
1681 pdqtemtieg lqptveyvvs vyaqnpsges qplvqtavtn idrpkglaft dvdvdsikia
1741 wespqgqvsr yrvtyssped gihelfpapd geedtaelqg lrpgseytvs vvalhddmes
1801 qpligtqsta ipapadlkft qvtptslsaq wtppnvqltg yrvrvtpkek tgpmkeinla
1861 pdsssvvvsg lmvatkyevs vyalkdtlts rpaqgvvttl envspprrar vtdatettit
1921 iswrtkteti tgfqvdavpa ngqtpiqrti kpdvrsytit glqpgtdyki ylytlndnar
1981 sspvvidast aidapsnlrf lattpnsllv swqpprarit gyiikyekpg spprevvprp
2041 rpgvteatit glepgteyti yvialknnqk sepligrkkt delpqlvtlp hpnlhgpeil
2101 dvpstvqktp fvthpgydtg ngiqlpgtsg qqpsvgqqmi feehgfrrtt ppttatpirh
2161 rprpyppnvg qealsqttis wapfqdtsey iischpvgtd eeplqfrvpg tstsatltgl
2221 trgatyniiv ealkdqqrhk vreevvtvgn svneglnqpt ddscfdpytv shyavgdewe
2281 rmsesgfkll cqclgfgsgh frcdssrwch dngvnykige kwdrqgengq mmsctclgng
2341 kgefkcdphe atcyddgkty hvgeqwqkey lgaicsctcf ggqrgwrcdn crrpggepsp
2401 egttgqsynq ysqryhqrtn tnvncpiecf mpldvqadre dsre
SEQ ID No. 29
ubiquitin A-52 residue ribosomal protein fusion product
trm|Q3MIH3
      1 mqifvktltg ktitleveps dtienvkaki qdkegippdq qrlifagkql edgrtlsdyn
    61 igkestlhlv lrlrggiiep slrglaqkyn cdkmicrkcy arlhpravnc rkkkcghtnn
  121 lrpkkkvk
SEQ ID No. 30
basement membrane specific heparin sulfate core protein
spt|P98160
      1 mgwraagall lalllhgrll avthglrayd glslpediet vtasqmrwth sylsddedml
    61 adsisgddlg sgdlgsgdfq mvyfralvnf trsieyspql edagsrefre vseavvdtle
  121 seylkipgdq vvsvvfikel dgwvfveldv gsegnadgaq iqemllrvis sgsvasyvts
  181 pqgfqfrrlg tvpqfpract eaefachsyn ecvaleyrcd rrpdcrdmsd elnceepvlg
  241 isptfsllve ttslpprpet timrqppvth apqpllpgsv rplpcgpqea acrnghcipr
  301 dylcdgqedc edgsdeldcg ppppcepnef pcgnghcalk lwrcdgdfdc edrtdeancp
  361 tkrpeevcgp tqfrcvstnm cipasfhcde esdcpdrsde fgcmppqvvt ppresigasr
  421 gqtvtftcva igvptpiinw rlnwghipsh prvtvtsegg rgtliirdvk esdqgaytce
  481 amnargmvfg ipdgvlelvp qrgpcpdghf ylehsaaclp cfcfgitsvc qstrrfrdqi
  541 rlrfdqpddf kgvnvtmpaq pgtpplsstq lqidpslhef qlvdlsrrfl vhdsfwalpe
  601 qflgnkvdsy ggslrynvry elargmlepv qrpdvvlmga gyrllsrght ptqpgalnqr
  661 qvqfseehwv hesgrpvqra ellqvlqsle avliqtvynt kmasvglsdi amdttvthat
  721 shgrahsvee crcpigysgl scescdahft rvpggpylgt csgcncngha sscdpvyghc
  781 lncqhntegp qcnkckagff gdamkatats crpcpcpyid asrrfsdtcf ldtdgqatcd
  841 acapgytgrr cescapgyeg npiqpggkcr pvngeivrcd ergsmgtsge acrcknnvvg
  901 rlcnecadgs fhlstrnpdg clkcfcmgvs rhctssswsr aqlhgaseep ghfsltnaas
  961 thttnegifs ptpgelgfss fhrllsgpyf wslpsrflgd kvtsyggelr ftvtqrsqpg
1021 stplhgqplv vlqgnniile hhvaqepspg qpstfivpfr eqawqrpdgq patrehllma
1081 lagidtllir asyaqqpaes rvsgismdva vpeetgqdpa leveqcscpp gyrgpscqdc
1141 dtgytrtpsg lylgtcercs chghseacep etgacqgcqh htegprceqc qpgyygdaqr
1201 gtpqdcqlcp cygdpaagqa ahtcfldtdg hptcdacspg hsgrhcerca pgyygnpsqg
1261 qpcqrdsqvp gpigcncdpq gsvssqcdaa gqcqckaqve gltcshcrph hfhlsasnpd
1321 gclpcfcmgi tqqcassayt rhlisthfap gdfqgfalvn pqrnsrltge ftvepvpega
1381 qlsfgnfaql ghesfywqlp etyqgdkvaa yggklrytls ytagpqgspl sdpdvgitgn
1441 nimlvasqpa lqgperrsye imfreefwrr pdgqpatreh llmaladlde lliratfssv
1501 plaasisavs levaqpgpsn rpraleveec rcppgyigls cqdcapgytr tgsglylghc
1561 elcecnghsd lchpetgacs qcqhnaagef celcapgyyg datagtpedc qpcacpltnp
1621 enmfsrtces lgaggyrcta cepgytgqyc eqcgpgyvgn psvqgggclp etnqaplvve
1681 vhparsivpq ggshslrcqv sgspphyfyw sredgrpvps gtqqrhqgse lhfpsvqpsd
1741 agvyictcrn lhqsntsrae llvteapskp itvtveeqrs gsvrpgadvt fictaksksp
1801 aytivwtrlh ngklptramd fngiltirnv qlsdagtyvc tgsnmfamdq gtatlhvgas
1861 gtlsapvvsi hppqltvqpg glaefrcsat gsptptlewt ggpggqlpak aqihggilrl
1921 paveptdqaq ylcrahssag qqvaravlhv hggggprvqv spertqvhag rtvrlycraa
1981 gvpsatitwr keggslppqa rsertdiatl lipaittada gfylcvatsp agtaqariqv
2041 vvlsasdasp ppvkiesssp svtegqtldl ncvvagsaha qvtwyrrggs lpphtqvhgs
2101 rlrlpqvspa dsgeyvcrve ngsgpkeasi tvsvlhgths gpsytpvpgs trpiriepss
2161 shvaegqtld lncvvpgqah aqvtwhkrgg slparhqthg sllrlhqvtp adsgeyvchv
2221 vgtsgpleas vlvtieasvi pgpippvrie sssstvaegq tldlscvvag qahaqvtwyk
2281 rggslparhq vrgsrlyifq aspadagqyv crasngmeas itvtvtgtqg anlaypagst
2341 qpiriepsss qvaegqtldl ncvvpgqsha qvtwhkrggs lpvrhqthgs llrlyqaspa
2401 dsgeyvcrvl gssvpleasv lvtiepagsv palgvtptvr iessssqvae gqtldlnclv
2461 agqahaqvtw hkrggslpar hqvhgsrlrl lqvtpadsge yvcrvvgssg tqeasvlvti
2521 qqrlsgshsq gvaypvries ssaslanght ldlnclvasq aphtitwykr ggslpsrhqi
2581 vgsrlripqv tpadsgeyvc hvsngagsre tslivtiqgs gsshvpsysp piriessspt
2641 vvegqtldln cvvarqpqai itwykrggsl psrhqthgsh lrlhqmsvad sgeyvcrann
2701 nidaleasiv isvspsagsp sapgssmpir iesssshvae getldlncvv pgqahaqvtw
2761 hkrggslpsh hqtrgsrlrl hhvspadsge yvcrvmgssg pleasvlvti easgssavhv
2821 papggappir iepsssrvae gqtldlkcvv pgqahaqvtw hkrggnlpar hqvhgpllrl
2881 nqvspadsge yscqvtgssg tleasvlvti epsspgpipa pglaqpiyie assshvtegq
2941 tldlncvvpg qahaqvtwyk rggslparhq thgsqlrlhl vspadsgeyv craasgpgpe
3001 qeasftvtvp psegssyrlr spvisidpps stvqqgqdas fkclihdgaa pislewktrn
3061 gelednvhis pngsiitivg trpsnhgtyr cvasnaygva qsvvnlsvhg pptvsvlpeg
3121 pvwvkvgkav tlecvsagep rssarwtris stpakleqrt yglmdshavl qissakpsda
3181 gtyvclaqna lgtaqkqvev ivdtgamapg apqvgaeeae ltveaghtat lrcsatgspa
3241 ptihwsklrs plpwqhrleg dtliiprvaq qdsgqyicna tspaghaeat iilhvesppy
3301 attvpehasv qagetvqlqc lahgtppltf qwsrvgsslp gratarnell hferaapeds
3361 gryrcrvtnk vgsaeafaql lvqgppgslp atsipagstp tvqvtpqlet ksigasvefh
3421 cavpsdrgtq lrwfkeggql ppghsvqdgv lriqnldqsc qgtyicqahg pwgkaqasaq
3481 lviqalpsvl inirtsvqtv vvghavefec lalgdpkpqv twskvgghlr pgivqsggvv
3541 riahvelada gqyrctatna agttqshvll lvqalpgism pqevrvpags aavfpciasg
3601 yptpdiswsk ldgslppdsr lennmlmlps vrpqdagtyv ctatnrqgkv kafahlqvpe
3661 rvvpyftqtp ysflplptik dayrkfeiki tfrpdsadgm llyngqkrvp gsptnlanrq
3721 pdfisfglvg grpefrfdag sgmatirhpt plalghfhtv tllrsltqgs livgdlapvn
3781 gtsqgkfqgl dlneelylgg ypdygaipka glssgfigcv relriqgeei vfhdlnltah
3841 gishcptcrd rpcqnggqch dsesssyvcv cpagftgsrc ehsqalhchp eacgpdatcv
3901 nrpdgrgytc rchlgrsglr ceegvtvttp slsgagsyla lpaltnthhe lrldvefkpl
3961 apdgvllfsg gksgpvedfv slamvgghle fryelgsgla vlrsaeplal grwhrvsaer
4021 lnkdgslrvn ggrpvlrssp gksqglnlht llylggveps vplspatnms ahfrgcvgev
4081 svngkrldlt ysflgsqgig qcydsspcer qpcqhgatcm pageyefqcl crdgfkgdlc
4141 eheenpcqlr epclhggtcq gtrclclpgf sgprcqqgsg hgiaesdwhl egsggndapg
4201 qygayfhddg flafpghvfs rslpevpeti elevrtstas glllwqgvev geagqgkdfi
4261 slglqdghlv fryqlgsgea rlvsedpind gewhrvtalr egrrgsiqvd geelvsgrsp
4321 gpnvavnakg svyiggapdv atltggrfss gitgcvknlv lhsarpgapp pqpldlqhra
4381 qagantrpcp s
SEQ ID No. 31
Versican
trm|Q59FG9
      1 ifppncnkpp skakmfinik silwmcstli vthalhkvkv gksppvrgsl sgkvslpchf
    61 stmptlppsy ntseflrikw skievdkngk dlkettvlva qngnikigqd ykgrvsvpth
  121 peavgdaslt vvkllasdag lyrcdvmygi edtqdtvslt vdgvvfhyra atsrytlnfe
  181 aaqkacldvg aviatpeqlf aayedgfeqc dagwladqtv rypiraprvg cygdkmgkag
  241 vrtygfrspq etydvycyvd hldgdvfhlt vpskftfeea akecenqdar latvgelqaa
  301 wrngfdqcdy gwlsdasvrh pvtvaraqcg ggllgvrtly rfenqtgfpp pdsrfdaycf
  361 kpkeattidl silaetasps lskepqmvsd rttpiiplvd elpviptefp pvgnivsfeq
  421 katvqpqait dslatklptp tgstkkpwdm ddyspsasgp lgkldiseik eevlqsttgv
  481 shyatdswdg vvedkqtqes vtqieqievg plvtsmeilk hipskefpvt etplvtarmi
  541 lesktekkmv stvselvttg hygftlgeed dedrtltvgs destlifdqi pevitvskts
  601 edtihthled lesysasttv splimpdnng ssmddweerq tsgriteefl gkylsttpfp
  661 sqhrteielf pysgdkilve gistviypsl qtemthrrer tetlipemrt dtytdeiqee
  721 itkspfmgkt eeevfsgmkl stslsepihv tessvemtks fdfptlitkl saeptevrdm
  781 eedftatpgt tkydenittv llahgtlsve aatvskwswd ednttskple stepsasskl
  841 ppallttvgm ngkdkdipsf tedgadeftl ipdstqkqle evtdediaah gkftirfqpt
  901 tstgiaekst lrdstteekv ppitstegqv yatmegsalg evedvdlskp vstvpqfaht
  961 seveglafvs ysstqeptty vdsshtipls vipktdwgvl vpsvpsedev lgepsqdilv
1021 idqtrleati spetmrttki tegttqeefp wkeqtaekpv palsstawtp keavtpldeq
1081 egdgsaytvs edelltgser vpvlettpvg kidhsysypp gavtehkvkt devvtltpri
1141 gpkvslspgp eqkyetegss ttgftsslsp fsthitqlme etttektsle didlgsglfe
1201 kpkatelief stikvtvpsd ittafssvdr lhttsafkps saitkkppli drepgeetts
1261 dmviigests hvppttledi vaketetdid reyfttsspp atqptrpptv edkeafgpqa
1321 lstpqppast kfhpdinvyi ievrenktgr msdlsvighp idseskedep cseetdpvhd
1381 lmaeilpefp diieidlyhs eeneeeeeec anatdvtttp svqyingkhl vttvpkdpea
1441 aearrgqfes vapsqnfsds sesdthpfvi aktelstavq pnestettes levtwkpety
1501 petsehfsgg epdvfptvpf heefesgtak kgaesvterd tevghqaheh tepvslfpee
1561 ssgeiaidge sqkiafarat evtfgeevek stsvtytpti vpssasayvs eeeavtlign
1621 pwpddllstk eswveatprq vvelsgsssi pitegsgeae ededtmftmv tdlsqrnttd
1681 tlitldtsri itesffevpa ttiypvseqp sakvvptkfv setdtsewis sttveekkrk
1741 eeegttgtas tfevysstqr sdqlilpfel espnvatssd sgtrksfmsl ttptqserem
1801 tdstpvftet ntlenlgaqt tehssihqpg vqeglttlpr spasvfmeqg sgeaaadpet
1861 ttvssfslnv eyaiqaekev agtlsphvet tfsteptglv lstvmdrvva enitqtsrei
1921 viserlgepn ygaeirgfst gfpleedfsg dfreystvsh piakeetvmm egsgdaafrd
1981 tqtspstvpt svhishisds egpsstmvst safpweefts saegsgeqlv tvsssvvpvl
2041 psavqkfsgt assiideglg evgtvneidr rstilptaev egtkapveke evkvsgtvst
2101 nfpqtiepak lwsrqevnpv rqeiesetts eeqiqeeksf espqnspate qtifdsqtft
2161 etelkttdys vlttkktysd dkemkeedts lvnmstpdpd anglesyttl peatekshff
2221 latalvtesi paehvvtdsp ikkeestkhf pkgmrptiqe sdtellfsgl gsgeevlptl
2281 ptesvnftev eqinntlyph tsqvestssd kiedfnrmen vakevgplvs qtdifegsgs
2341 vtsttlieil sdtgaegptv aplpfstdig hpqnqtvrwa eeiqtsrpqt iteqdsnkns
2401 staeinettt sstdflaray gfemakefvt sapkpsdlyy epsgegsgev divdsfhtsa
2461 ttqatrqess ttfvsdgsle khpevpsaka vtadgfptvl vmlplhseqn ksspdptstl
2521 sntvsyerst dgsfqdrfre fedstlkpnr kkpteniiid ldkedkdlil titestilei
2581 1peltsdknt iididhtkpv yedilgmqtd idtevpseph dsndesndds tqvgeiyeaa
2641 vnlslteetf egsadvlasy tqathdesmt yedrsqldhm gfhfttgipa psteteldvl
2701 1ptatslpip rksatvipei egikaeakal ddmfesstls dgqaiadqse iiptlgqfer
2761 tqeeyedkkh agpsfqpefs sgaeealvdh tpylsiatth lmdqsvtevp dvmegsnppy
2821 ytdttlayst faklssqtps spltiysgse asghteipqp salpgidvgs svmspqdsfk
2881 eihvnieatf kpsseeylhi teppslspdt klepseddgk pelleemeas pteliavegt
2941 eilqdfqnkt dgqvsgeaik mfptiktpea gtvittadei elegatqwph stsasatygv
3001 eagvvpwlsp qtserptlss speinpetqa alirgqdsti aaseqqvaar ildsndqatv
3061 npvefnteva tppfsllets netdfligin eesvegtaiy lpgpdrckmn pclnggtcyp
3121 tetsyvctcv pgysgdqcel dfdechsnpc rngatcvdgf ntfrqclps yvgalceqdt
3181 etcdygwhkf qgqcykyfah rrtwdaaere crlqgahlts ilsheeqmfv nrvghdyqwi
3241 glndkmfehd frwtdgstlq yenwrpnqpd sffsagedcv viiwhengqw ndvpcnyhlt
3301 ytckkgtvac gqppvvenak tfgkmkprye insliryhck dgfiqrhlpt irclgngrwa
3361 ipkitcmnps ayqrtysmky fknsssakdn sintskhdhr wsrrwqesrr
SEQ ID No. 32
gamma-glutamyl hydrolase
spt|Q92820
      1 maspgcllcv lglllcgaas lelsrphgdt akkpiigilm qkcrnkvmkn ygryyiaasy
    61 vkylesagar vvpvrldlte kdyeilfksi ngilfpggsv dlrrsdyakv akifynlsiq
  121 sfddgdyfpv wgtclgfeel sllisgecll tatdtvdvam pinftggqlh srmfqnfpte
  181 lllslavepl tanfhkwsls vknftmnekl kkffnvlttn tdgkiefist megykypvyg
  241 vqwhpekapy ewknldgish apnavktafy laeffvnear knnhhfkses eeekaliyqf
  301 spiytgniss fqqcyifd
SEQ ID No. 33
lysyl oxidase-like 2
spt|Q9Y4K0
      1 merplcshlc sclamlalls plslaqydsw phypeyfqqp apeyhqpqap anvakiqlrl
    61 agqkrkhseg rvevyydgqw gtvcdddfsi haahvvcrel gyveakswta sssygkgegp
  121 iwldnlhctg neatlaacts ngwgvtdckh tedvgvvcsd kripgfkfdn slinqienln
  181 iqvedirira ilstyrkrtp vmegyvevke gktwkqicdk hwtaknsrvv cgmfgfpger
  241 tyntkvykmf asrrkqrywp fsmdctgtea hisscklgpq vsldpmknvt cenglpavvs
  301 cvpgqvfspd gpsrfrkayk peqplvrlrg gayigegrve vlkngewgtv cddkwdlvsa
  361 svvcrelgfg sakeavtgsr lgqgigpihl neiqctgnek siidckfnae sqgcnheeda
  421 gvrcntpamg lqkklringg rnpyegrvev lverngslvw gmvcgqnwgi veamvvcrql
  481 glgfasnafq etwywhgdvn snkvvmsgvk csgtelslah crhdgedvac pqggvqygag
  541 vacsetapdl vinaemvqqt tyledrpmfm lqcameencl sasaaqtdpt tgyrrllrfs
  601 sqihnngqsd frpkngrhaw iwhdchrhyh smevfthydl lnlngtkvae ghkasfcled
  661 tecegdiqkn yecanfgdqg itmgcwdmyr hdidcqwvdi tdvppgdylf qvvinpnfev
  721 aesdysnnim kcrsrydghr iwmynchigg sfseetekkf ehfsgllnnq lspq
SEQ ID No. 34
syndecan-4
spt|P31431
      1 maparlfall lffvggvaes iretevidpq dllegryfsg alpddedvvg pgqesddfel
    61 sgsgdlddle dsmigpevvh plvpldnhip eragsgsqvp tepkkleene vipkrispve
  121 esedvsnkvs msstvqgsni fertevlaal ivggivgilf avflilllmy rmkkkdegsy
  181 dlgkkpiykk aptnefya
SEQ ID No. 35
hnRNP A2/131
spt|22626
      1 mektletvpl erkkrekeqf rklfigglsf etteeslrny yeqwgkltdc vvmrdpaskr
    61 srgfgfvtfs smaevdaama arphsidgry vepkravare esgkpgahvt vkklfvggik
  121 edteehhlrd yfeeygkidt ieiitdrqsg kkrgfgfvtf ddhdpvdkiv lqkyhtingh
  181 naevrkalsr qemqevqssr sgrggnfgfg dsrggggnfg pgpgsnfrgg sdgygsgrgf
  241 gdgyngyggg pgggnfggsp gygggrggyg gggpgygnqg ggygggydny gggnygsgny
  301 ndfgnynqqp snygpmksgn fggsrnmggp ygggnygpgg sggsggyggr sry
SEQ ID No. 36
MCFD2
trm|Q68D61
      1 mehlegvink peaemspqel qlhyfkmhdy dgnnlldgle lstaithvhk eegseqaplm
    61 sedeliniid gvlrdddknn dgyidyaefa kslq
SEQ ID No. 37
Niemann-Pick disease, type C2 variant
trm|Q53HV6
      1 mrflaatfll lalstaaqae pvqfrdcgsv dgvikevnvs pcptqpcqls kgqsysvnvt
    61 ftsnvqskss kavvhgilmg vpvpfpipep dgcksgincp iqkdktysyl nklpvkseyp
  121 siklvvewql qddknqslfc weipvgivsh l
SEQ ID No. 38
14-3-3 protein epsilon (14-3-3E)
spt|P62258
      1 mddredlvyq aklaeqaery demvesmkkv agmdveltve ernllsvayk nvigarrasw
    61 riissieqke enkggedklk mireyrqmve telkliccdi ldvldkhlip aantgeskvf
  121 yykmkgdyhr ylaefatgnd rkeaaenslv aykaasdiam telppthpir lglalnfsvf
  181 yyeilnspdr acrlakaafd daiaeldtls eesykdstli mqllrdnitl wtsdmqgdge
  241 eqnkealqdv edenq
SEQ ID No. 39
60S aqdic ribosomal protein P2 (NY-REN-44 antigen)
spt|P05387
      1 mryvasylla alggnsspsa kdikkildsv gieadddrin kviselngkn iedviaggig
    61 klasvpagga vaysaapgsa apaagsapaa aeekkdekke eseesdddmg fglfd
SEQ ID No. 40
Agrin
spt|O00468
      1 magrshpgpl rpllpllvva acvlpgaggt cperalerre eeanvvltgt veeilnvdpv
    61 qhtysokvry wrylkgkdlv areslldggn kvvisgfgdp licdnqvstg dtriffvnpa
  121 ppylwpahkn elmlnsslmr itlrnleeve fcvedkpgth ftpvpptppd acrgmlcgfg
  181 avcepnaegp grascvckks pcpsvvapvc gsdastysne celqraqcsq qrrirllsrg
  241 pcgsrdpcsn vtcsfgstca rsadgltasc lcpatcrgap egtvcgsdga dypgeoqllr
  301 racargenvf kkfdgpcdpc qgalpdpsrs crvnprtrrp emllrpescp arqapvcgdd
  361 gvtyendcvm grsgaargll lqkvrsgqcq grdqcpeper fnavclsrrg rproscdrvt
  421 cdgayrpvca qdgrtydsdc wrqqaecrqq raipskhqgp cdqapspclg vqcafgatca
  481 vkngqaacec lqacsslydp vcgsdgvtyg saceleatac tlgreiqvar kgpcdrcgqc
  541 rfgalceaet grcvcpsecv alaqpvcgsd ghtypsecml hvhacthqis lhvasagpce
  601 tcgdavcafg avcsagqcvc prcehpppgp vcgsdgvtyg sacelreaac lqqtqieear
  661 agpceqaecg sggsgsgedg dceqelcrqr ggiwdedsed gpcvcdfscq svpgspvcgs
  721 dgvtystece lkkarcesqr glyvaaqgac rgptfaplpp vaplhcaqtp ygccqdnita
  781 argvglagcp sacqcnphgs yggtcdpatg qcscrpgvgg lrcdrcepgf wnfrgivtdg
  841 rsgctpcscd pqgavrddce qmtglcsckp gvagpkcgqc pdgralgpag ceadasapat
  901 caemrcefga rcveesgsah cvcpmltcpe anatkvcgsd gvtygnecql ktiacrqglq
  961 isiqslgpcq eavapsthpt sasvtvttpg lllsqalpap pgalplapss tahsqttppp
1021 ssrprttasv prttvwpvlt vpptapspap slvasafges gstdgssdee lsgdqeasgg
1081 gsgglepleg ssvatpgppv erascynsal gccsdgktps ldaegsncpa tkvfqgvlel
1141 egvegqelfy tpemadpkse lfgetarsie stlddlfrns dvkkdfrsvr lrdlgpgksv
1201 raivdvhfdp ttafrapdva rallrqiqvs rrrslgvrrp lqehvrfmdf dwfpafitga
1261 tsgaiaagat arattasrlp ssavtpraph pshtsqpvak ttaapttrrp pttapsrvpg
1321 rrppapqqpp kpcdsqpcfh ggtcqdwalg ggftcscpag rggavcekvl gapvpafegr
1381 sflafptlra yhtlrlalef ralepqglll yngnargkdf lalalldgry qlrfdtgsgp
1441 avltsavpve pgqwhrlels rhwrrgtlsv dgetpvlges psgtdglnld tdlfvggvpe
1501 dqaavalert fvgaglrgci rlldvnnqrl elgigpgaat rgsgvgecgd hpclpnpchg
1561 gapcqnleag rfhcqcppgr vgptcadeks pcqpnpchga apervlpegg aqcecplgre
1621 gtfcqtasgq dgsgpfladf ngfshlelrg lhtfardlge kmalevvfla rgpsglllyn
1681 gqktdgkgdf vslalrdrrl efrydlgkga avirsrepvt lgawtrvsle rngrkgalry
1741 gdgprvlges pvphtvinlk eplyvggapd fsklaraaav ssgfdgaiql vslggrqllt
1801 pehvlrqvdv tsfaghpctr asghpclnga scvpreaayv qcpggfsgph cekglveks
1861 agdvdtlafd grtfveylna vtesekalqs nhfelslrte atqglvlwsg kateradyva
1921 laivdghlql synlgsqpvv lrstvpvntn rwlrvvahre qregslqvgn eapvtgsspl
1981 gatqldtdga lwlgglpelp vgpalpkayg tgfvgclrdv vvgrhplhll edavtkpelr
2041 pcptp
SEQ ID No. 41
Alpha-actinin-1
spt|P12814
      1 mdhydsqqtn dymqpeedwd rdllldpawe kqqrktftaw cnshlrkagt qienieedfr
    61 dglklmllle visgerlakp ergkmrvhki snvnkaldfi askgvklvsi gaeeivdgnv
  121 kmtlgmiwti ilrfaiqdis veetsakegl llwcqrktap yknvniqnfh iswkdglgfc
  181 alihrhrpel idygklrkdd pltnlntafd vaekyldipk mldaedivgt arpdekaimt
  241 yvssfyhafs gaqkaetaan rickvlavnq eneqlmedye klasdllewi rrtipwlenr
  301 vpentmhamq qkledfrdyr rlhkppkvqe kcqleinfnt lqtklrlsnr pafmpsegrm
  361 vsdinnawgc legvekgyee wllneirrle rldhlaekfr qkasiheawt dgkeamlrqk
  421 dyetatlsei kallkkheaf esdlaahqdr vegiaaiage lneldyydsp svnarcqkic
  481 dqwdnlgalt qkrrealert eklletidql yleyakraap fnnwmegame dlqdtfivht
  541 ieeiqgltta heqfkatlpd adkerlailg ihnevskivq tyhvnmagtn pyttitpqei
  601 ngkwdhvrql vprrdqalte eharqqhner lrkqfgaqan vigpwiqtkm eeigrisiem
  661 hgtledqlsh lrqyeksivn ykpkidqleg dhqliqeali fdnkhtnytm ehirvgweql
  721 lttiartine venqiltrda kgisqeqmne frasfnhfdr dhsgtlgpee fkaclislgy
  781 digndpqgea efarimsivd pnrlgvvtfq afidfmsret adtdtadqvm asfkilagdk
  841 nyitmdelrr elppdqaeyc iarmapytgp dsvpgaldym sfstalyges dl
SEQ ID No. 42
Alpha-actinin-4
spt|O43707
      1 mvdyhaanqs yqygpssagn gaggggsmgd ymaqeddwdr dllldpawek qqrktftawc
    61 nshlrkagtq ienidedfrd glklmlllev isgerlpkpe rgkmrvhkin nvnkaldfia
  121 skgvklvsig aeeivdgnak mtlgmiwtii lrfaiqdisv eetsakegll lwcqrktapy
  181 knvnvqnfhi swkdglafna lihrhrpeli eydklrkddp vtnlnnafev aekyldipkm
  241 ldaedivnta rpdekaimty vssfyhafsg aqkaetaanr ickvlavnqe nehlmedyek
  301 lasdllewir rtipwledry pqktiqemqq kledfrdyrr vhkppkvqek cqleinfntl
  361 qtklrlsnrp afmpsegkmv sdinngwqhl egaekgyeew llneirrler ldhlaekfrq
  421 kasiheawtd gkeamlkhrd yetatlsdik alirkheafe sdlaahqdry egiaaiagel
  481 neldyydshn vntrcqkicd qwdalgslth srrealekte kgleaidglh leyakraapf
  541 nnwmesamed lqdmfivhti eeieglisah dqfkstlpda drereailai hkeaqriaes
  601 nhiklsgsnp yttvtpqiin skwekvqqlv pkrdhallee qskqqsnehl rrqfasqanv
  661 vgpwiqtkme eigrisiemn gtledqlshl kgyersivdy kpnldllegq hgligealif
  721 dnkhtnytme hirvgweqll ttiartinev enqiltrdak gisqegmqef rasfnhfdkd
  781 hggalgpeef kaclislgyd vendrqgeae fnrimslvdp nhsglvtfqa fidfmsrett
  841 dtdtadqvia sfkvlagdkn fitaeelrre lppdgaeyci armapycopd avpgaldyks
  901 fstalygesd l
SEQ ID No. 43
Beta-2-microglobulin
spt|P61769
      1 msrsvalavl allslsglea iqrtpkiqvy srhpaengks nflncyvsgf hpsdievdll
    61 kngeriekve hsdlsfskdw sfyllyytef tptekdeyac rvnhvtlsqp kivkwdrdm
SEQ ID No. 44
C4B1 (Complement component C4B)
trm|Q6U2E9
      1 mrllwgliwa ssfftlslqk prlllfspsv vhlgvplsvg vqlqdvprgq vvkgsvflrn
    61 psrnnvpcsp kvdftlsser dfallslqvp lkdakscglh qllrgpevql vahspwlkds
  121 lsrttniggi nllfssrrgh lflqtdqpiy npgqrvryry faldqkmrps tdtitvmven
  181 shglrvrkke vympssifqd dfvipdisep gtwkisarfs dglesnsstq fevkkyvlpn
  241 fevkitpgkp yiltvpghld emqldigary iygkpvcova yvrfgllded gkktffrgle
  301 sqtklvngqs hislskaefq daleklnmgi tdlqglrlyv aaaiiespgg emeeaeltsw
  361 yfvsspfsld lsktkrhlvp gapfllqalv remsgspasg ipvkvsatvs spgsvpevqd
  421 iqqntdgsgq vsipiiipqt iselqlsysa gsphpaiarl tvaappsggp gflsierpds
  481 rpprvgdtln lnlravgsga tfshyyymil srgqivfmnr epkrtltsys vfvdhhlaps
  541 fyfvafyyhg dhpvanslry dvgagacegk lelsvdgakq yrngesvklh letdslalva
  601 lgaldtalya agskshkpin mgkvfeamns ydlgcgpggg dsalqvfgaa glafsdgdqw
  661 tlsrkrlscp kekttrkkrn vnfqkainek lgqyasptak rocqdgvtrl pmmrsceqra
  721 arvqqpdcre pflsccqfae slrkksrdkg qaglgralei lqeedlided dipvrsffpe
  781 nwlwrvetvd rfqiltlwlp dslttweihg lslsktkglc vatpvqlrvf refhlhlrlp
  841 msvrrfeqle lrpvlynyld knitvsvhvs pveglclagg gglaqqvlvp agsarpvafs
  901 vvptaatays lkvvargsfe fpvgdayskv lqiekegaih reelvyelnp ldhrgrtlei
  961 pgnsdpnmip dgdfnsyvry tasdpldtlg segalspggv asllrlprgc geqtmiylap
1021 tlaasryldk teqwstlppe tkdhavdliq kgymriqqfr kadgsyaawl srgsstwlta
1081 fvlkvlslaq eqvggspekl getsnwllsq qqadgsfqdl spvihrsmqg glvgndetva
1141 ltafvtialh hglavfqdeg aeplkqrvea siskassflg ekasagllga haaaitayal
1201 tltkapadlr gvahnnlmam agetgdnlyw gsvtgsgsna vsptpaprnp sdpmpqapal
1261 wiettayall hlllhegkae madqaaawlt rqgsfqggfr stqdtviald alsaywiash
1321 tteerglnvt lsstgrngfk shalqlnnrq irgleeelqf slgskinvkv ggnskgtlkv
1381 lrtynvldmk nttcqdlqie vtvkghveyt meanedyedy eydelpakdd pdaplqpvtp
1441 lqlfegrrnr rrreapkvve eqesrvhytv ciwrngkvgl sgmaiadvtl lsgfhalrad
1501 lekltslsdr yvshfetegp hvllyfdsvp tsrecvgfea vqevpvglvg pasatlydyy
1561 nperrcsvfy gapsksrlla ticsaevcqc aegkcprqrr alerglqded gyrmkfacyy
1621 prveygfqvk vlredsraaf rlfetkitqv lhftkdvkaa anqmrnflvr ascrlrlepg
1681 keylimgldg atydleghpq ylldsnswie empserlcrs trqraacaql ndflgeygtq
1741 gcqv
SEQ ID No. 45
Cadherin 2
spt|P19022
      1 mcriagalrt llpllaallq asveasgeia lcktgfpedv ysavlskdvh eggpllnvkf
    61 sncngkrkvq yessepadfk vdedgmvyav rsfplsseha kfliyaqdke tgekwqvavk
  121 lslkptltee svkesaevee ivfprqfskh sghlgrqkrd wvippinlpe nsrgpfpqel
  181 vrirsdrdkn lslrysvtgp gadqpptgif iinpisgqls vtkpldreqi arfhlrahav
  241 dingnqvenp idivinvidm ndnrpeflhq vwngtvpegs kpgtyvmtvt aidaddpnal
  301 ngmlryrivs qapstpspnm ftinnetgdi itvaagldre kvqqytliiq atdmegnpty
  361 glsntatavi tvtdvndnpp eftamtfyge vpenrvdiiv anitvtdkdq phtpawnavy
  421 risggdptgr faiqtdpnsn dglvtvvkpi dfetnrmfvl tvaaenqvpl akgighppqs
  481 tatvsvtvid vnenpyfapn pkiirgeegl hagtmlttft aqdpdrymqg nirytklsdp
  541 anwlkidpvn gqittiavld respnvknni ynatflasdn gippmsgtgt lqiylldind
  601 napqvlpgea etcetpdpns initaldydi dpnagpfafd lplspvtikr nwtitringd
  661 faqlnlkikf leagiyevpi iitdsgnppk snisilrvkv cqcdsngdct dvdrivgagl
  721 gtgaiiaill ciiillilvl mfvvwmkrrd kerqakqlli dpeddvrdni lkydeeggge
  781 edgdydlsql qqpdtvepda ikpvgirrmd erpihaepqy pvrsaaphpg digdfinegl
  841 kaadndptap pydsllvfdy egsgstagsl sslnssssgg egdydylndw gprfkkladm
  901 ygggdd
SEQ ID No. 46
Calreticulin (CRP55)
trm|Q53G71
      1 lglavaepav yfkeqfldgd gwtsrwiesk hksdfgkfvl ssgkfygdee kdkglqtsqd
    61 arfyalsasf epfsnkgqtl vvqftvkheq nidcgggyvk lfpnsldqtd mhgdseynim
  121 fgpdicgpgt kkvhvifnyk gknvlinkdi rckddefthl ytlivrpdnt yevkidnsqv
  181 esgsleddwd flppkkikdp daskpedwde rakiddptds kpedwdkpeh ipdpdakkpe
  241 dwdeemdgew eppviqnpey kgewkprgid npdykgtwih peidnpeysp dpsiyaydnf
  301 gvlgldlwqv ksgtifdnfl itndeayaee fgnetwgvtk aaekqmkdkq deeqrlkeee
  361 edkkrkeeee aedkgddedk dedeedeedk eedeeedvpg qakdel
SEQ ID No. 47
Cathepsin C
trm|Q8WY99
      1 mgagpsllla alllllsgdg avrcdtpanc tyldllgtwv fqvgssgsqr dvncsvmgpq
    61 ekkvvvylqk ldtayddlgn sghftiiynq gfeivindyk wfaffkykee gskvttycne
  121 tmtgwvhdvl grnwacftgk kvgtasenvy vntahlknsq ekysnrlyky dhnfvkaina
  181 iqkswtatty meyetltlgd mirrsgghsr kiprpkpapl taeiqqkilh lptswdwrnv
  241 hginfvspvr nqascgscys fasmgmlear iriltnnsqt pilspqevvs ysqyaggceg
  301 gfpyliagky aqdfglveea cfpytgtdsp ckmkedcfry ysseyhyvgg fyggcnealm
  361 klelvhhgpm avafevyddf lhykkgiyhh tglrdpfnpf eltnhavllv gygtdsasgm
  421 dywivknswg tgwgengyfr irrgtdecai esiavaatpi pkl
SEQ ID No. 48
Cathepsin Z
trm|Q5U000
      1 marrgpgwrp llllvllaga aqgglyfrrg qtcyrplrgd glaplgrsty prpheylspa
    61 dlpkswdwrn vdgvnyasit rnghipqycg scwahastsa madrinikrk gawpstllsv
  121 qnvidcgnag sceggndlsv wdyahqhgip detcnnyqak dqecdkfnqc gtcnefkech
  181 airnytlwry gdygslsgre kmmaeiyang piscgimate rlanytggiy aeyqdttyin
  241 hvvsvagwgi sdgteywivr nswgepwger gwlrivtsty kdgkgaryni aieehctfgd
  301 piv
SEQ ID No. 49
CDNA FLJ45706 fis, clone FEBRA2028457, highly similar to Nucleolin
trm|Q6ZS99
      1 mvklakagkn qgdpkkmapp pkeveedsed eemsedeedd edddddeedd seeeamettp
    61 akgkkaakvv pvkakgakng knakkedsde eedddseede eddededede deiepaamka
  121 aaaapasede ddeddedded ddddeddedd deddeeeeee eeeepvkeap gkrkkemakq
  181 kaapeakkqk vegtepttaf nlfvgnlnfn ksapelktgi sdvfakndla vvdvrigmtr
  241 kfgyvdfesa edlekalelt glkvfgneik lekpkgkdsk kerdartlla knlpykvtqd
  301 elkevfedaa eirlvskdgn skgiayiefk teadaektfe ekqgteidgr sislyytgek
  361 gqnqdyrggk nstwsgeskt lvlsnlsysa teetlqevfe katfikvpqn qngkskgyaf
  421 iefasfedak ealnscnkre iegrairlel qgprgspnar sqpsktlfvk glsedtteet
  481 lkesfdgsvr arivtdretg sskgfgfvdf nseedakaak eamedgeidg nkvtldwakp
  541 kgeggfggrg ggrggfggrg ggrggrggfg grgrggfggr ggfrggrggg gdhkpqgkkt
  601 kfe
SEQ ID No. 50
Chaperonin 10-related protein
trm|Q9UNM1
      1 agqafrkflp lfdrvlvers aaetvtkggi mlpeksqgkv lqatvvavgs gskgkggeiq
    61 pvsvkvgdkv llpeyggtkv vlddkdyflf rdgdilg
SEQ ID No. 51
Cofilin-129
spt|P23528
      1 masgvaysdg vikvfndmkv rksstpeevk krkkavlfcl sedkkniile egkeilvgdv
    61 gqtvddpyat fvkmlpdkdc ryalydatye tkeskkediv fifwapesap lkskmiyass
  121 kdaikkkltg ikhelqancy eevkdrctla eklggsavis legkpl
SEQ ID No. 52
Collagen alpha-1 (V) chain
spt|P20908
      1 mdvhtrwkar salrpgapll ppllllllwa pppsraaqpa dllkvldfhn lpdgitkttg
    61 fcatrrsskg pdvayrvtkd aqlsaptkql ypasafpedf silttvkakk gsqaflvsiy
  121 neqgiqqigl elgrspvfly edhtgkpgpe dyplfrginl sdgkwhrial svhkknvtli
  181 ldckkkttkf ldrsdhpmid ingiivfgtr ildeevfegd iqqllfvsdh raaydycehy
  241 spdcdtavpd tpqsqdpnpd eyytegdgeg etyyyeypyy edpedlgkep tpskkpveaa
  301 kettevpeel tptpteaapm petsegagke edvgigdydy vpsedyytps pyddltygeg
  361 eenpdqptdp gagaeiptst adtsnssnpa pppgegaddl egefteetir nldenyydpy
  421 ydptsspsei gpgmpanqdt iyegiggprg ekgqkgepai iepgmliegp pgpegpaglp
  481 gppgtmgptg qvgdpgergp pgrpglpgad glpgppgtml mlpfrfgggg dagskgpmvs
  541 aqesqaqail qqarlalrgp agpmgltgrp gpvgppgsgg lkgepgdvgp qgprgvqgpp
  601 gpagkpgrrg ragsdgargm pgqtgpkgdr gfdglaglpg ekghrgdpgp sgppgppgdd
  661 gergddgevg prglpgepgp rgllgpkgpp gppgppgvtg mdgqpgpkgn vgpqgepgpp
  721 gqqgnpgaqg lpgpqgaigp pgekgplgkp glpgmpgadg ppghpgkegp pgekggqgpp
  781 gpqgpigypg prgvkgadgi rglkgtkgek gedgfpgfkg dmgikgdrge igppgprged
  841 gpegpkgrgg pngdpgplgp pgekgklgvp glpgypgrqg pkgsigfpgf pgangekggr
  901 gtpgkpgprg qrgptgprge rgprgitgkp gpkgnsggdg pagppgergp ngpqgptgfp
  961 gpkgppgppg kdglpghpgq rgetgfqgkt gppgppgvvg pqgptgetgp mgerghpgpp
1021 gppgecolpg lagkegtkgd pgpaglpgkd gppglrgfpg drglpgpvga lglkgnegpp
1081 gppgpagspg ergpagaagp igipgrpgpq gppgpagekg apgekgpqgp agrdglqgpv
1141 glpgpagpvg ppgedgdkge igepgqkgsk gdkgeqgppg ptgpqgpigq pgpsgadgep
1201 gprgqqglfg qkgdegprgf pgppgpvglq glpgppgekg etgdvgqmgp pgppgprgps
1261 gapgadgpqg ppggignpga vgekgepgea gepglpgegg ppgpkgerge kgesgpsgaa
1321 gppgpkgppg ddgpkgspgp vgfpgdpgpp gepgpagqdg ppgdkgddge pgqtgspgpt
1381 gepgpsgppg krgppgpagp egrqgekgak geaglegppg ktgpigpqga pgkpgpdglr
1441 gipgpvgeqg lpgspgpdgp pgpmgppglp glkgdsgpkg ekghpgligl igppgeggek
1501 gdrglpgpqg ssgpkgeggi tgpsgpigpp gppglpgppg pkgakgssgp tgpkgeaghp
1561 gppgppgppg eviqplpiqa srtrrnidas qllddgngen yvdyadgmee ifgslnslkl
1621 eieqmkrplg tqqnpartck dlqlchpdfp dgeywvdpnq gcsrdsfkvy cnftaggstc
1681 vfpdkksega ritswpkenp gswfsefkrg kllsyvdaeg npvgvvqmtf lrllsasahq
1741 nvtyhcyqsv awqdaatgsy dkalrflgsn deemsydnnp yiralvdgca tkkgyqktvl
1801 eidtpkveqv pivdimfndf geasqkfgfe vgpacfmg
SEQ ID No. 53
Collagen alpha-1 (VI) chain
spt|P12109
      1 mraarallpl llqacwtaaq depetprava fqdcpvdlff vldtsesval rlkpygalvd
    61 kvksftkrfi dnlrdryyrc drnlvwnaga lhysdeveii qgltrmpggr dalkssvdav
  121 kyfgkgtytd caikkgleql lvggshlken kylivvtdgh plegykepcg gledavneak
  181 hlgvkvfsva itpdhleprl siiatdhtyr rnftaadwgq srdaeeaisq tidtivdmik
  241 nnveqvccsf ecqpargppg lrgdpgfege rgkpglpgek geagdpgrpg dlgpvgyqgm
  301 kgekgsrgek gsrgpkgykg ekgkrgidgv dgvkgemgyp glpgckgspg fdgicoppgp
  361 kgdpgafglk gekgepgadg eagrpgssgp sgdegcmgep gppgekgeag degnpgpdga
  421 pgerggpger gprgtpgtrg prgdpgeagp qgdggregpv gvpgdpgeag pigpkgyrgd
  481 egppgsegar gapgpagppg dpglmgerge dgpagngteg fpgfpgypgn rgapgingtk
  541 gypglkgdeg eagdpgddnn diaprgvkga kgyrgpegpq gppghqgppg pdeceildii
  601 mkmcscceck cgpidllfvl dssesiglqn feiakdfvvk vidrlsrdel vkfepgqsya
  661 gvvqyshsqm gehvslrsps irnvqelkea ikslqwmagg tftgealqyt rdqllppspn
  721 nrialvitdg rsdtqrdttp lnvlcspgiq vvsvgikdvf dfipgsdqln viscqglaps
  781 corpglslvk enyaelleda flknvtaqic idkkcpdytc pitfsspadi tilldgsasv
  841 gshnfdttkr fakrlaerfl tagrtdpand vrvavvqysg tgqqrperas lqflqnytal
  901 asavdamdfi ndatdvndal gyvtrfyrea ssgaakkrll lfsdgnsqga tpaaiekavq
  961 eaqragieif vvvvgrqvne phirvlvtgk taeydvayge shlfrvpsyq allrgvfhqt
1021 vsrkvalg
SEQ ID No. 54
Collagen alpha-1 (XII) chain
spt|Q99715
      1 mrsrlppala algaalllss ieaevdppsd lnfkiident vhmswakpvd pivgyritvd
    61 pttdgptkef tlsasttetl lselvpetey vvtitsydev eesvpvigql tiqtgsstkp
  121 vekkpgktei qkcsysawtd lvflvdgsws vgrnnfkyil dfiaalvsaf digeektrvg
  181 vvqyssdtrt efnlnqyyqr dellaaikki pykggntmtg daidylvknt ftesagarvg
  241 fpkvaiiitd gksqdeveip arelrnvgve vfslgikaad akelkgiast pslnhvfnva
  301 nfdaivdiqn eiisqvcsgv deqlgelvsg eevveppsnl iamevsskyv klnwnpspsp
  361 vtgykviltp mtagsrqhal svgpqtttls vrdlsadtey gisysamkgm tssepisime
  421 ktgpmkvqve csrgvdikad ivflvdgsys igianfvkvr aflevlvksf eispnrvqis
  481 lvqysrdpht eftlkkftkv ediieaintf pyrggstntg kamtyvreki fvpskgsrsn
  541 vpkvmilitd gkssdafrdp aiklrnsdve ifavgvkdav rseleaiasp paethvftve
  601 dfdafqrisf eltqsiclri egelaaikkk ayvppkdlsf sevtsygfkt nwspagenvf
  661 syhitykeaa gddevtvvep asstsvvlss lkpetlylvn vtaeyedgfs iplageette
  721 evkgaprnlk vtdettdsfk itwtqapgry lryriiyrpv aggesrevtt ppnqrrrtle
  781 nlipdtkyev svipeyfsgp gtpltgnaat eevrgnprdl rvsdpttstm klswsgapgk
  841 vkqylvtytp vaggetqevt vrgdttntvl qglkegtqya lsvtalyasg agdalfgegt
  901 tleergspqd lvtkditdts igaywtsapg mvrgyrvswk slyddvdtge knlpedaiht
  961 mienlqpetk yrisvfatys sgegepltgd attelsqdsk tlkvdeeten tmrvtwkpap
1021 gkvvnyrvvy rphgrgkqmv akvpptvtst vlkrlqpqtt yditvlpiyk mgegklrqgs
1081 gttasrfksp rnlktsdptm ssfrvtwepa pgevkgykvt fhptgddrrl gelvvgpydn
1141 tvvleelrag ttykvnvfgm fdggessplv gqemttlsdt tvmpilssgm ecltraeadi
1201 vllvdgswsi granfrtvrs fisrivevfd igpkrvqial aqysgdprte wqlnahrdkk
1261 sllqavanlp ykggntltgm alnfirqqnf rtqagmrpra rkigvlitdg ksqddveap
1321 kklkdegvel faigiknade velkmiatdp ddthaynvad feslsrivdd ltinlcnsvk
1381 gpgdleapsn lviserthrs frvswtppsd svdrykveyy pvsggkrgef yvsrmetstv
1441 lkdlkpetey vvnvysvved eyseplkgte ktlpvpvvsl niydvgpttm hvqwqpvgga
1501 tgyilsykpv kdteptrpke vrlgptvndm qltdlvpnte yavtvgavlh dltsepvtvr
1561 evtlplprpq dlklrdvths tmnvfwepvp gkvrkyivry ktpeedvkev evdrsetsts
1621 lkdlfsqtly tvsysavhde gesppvtaqe ttrpvpaptn lkitevtseg frgtwdhgas
1681 dvslyritwa pfgssdkmet ilngdentiv fenlnpntiy evsitaiypd esesddligs
1741 ertlpilttq apksgprnlq vynatsnslt vkwdpasgry qkyrityqps tgegneqttt
1801 iggrqnsvvl qklkpdtpyt itvsslypdg eggrmtgrgk tkpintvrnl rvydpststl
1861 nvrwdhaegn prqyklfyap aaggpeelvp ipgntnyail rnlqpdtsyt vtvvpvyteg
1921 dggrtsdtgr tlmrglarnv qvynptpnsl dvrwdpapgp vlqyrvvysp vdgtrpsesi
1981 vvpgntrmvh lerlipdtly svnlvalysd gegnpspaqg rtlprsgprn lrvfgettns
2041 lsvawdhadg pvqqyriiys ptvgdpidey ttvpgrrnnv ilqplqpdtp ykitviavye
2101 dgdgghltgn grtvgllppq nihisdewyt rfrvswdpsp spvlgykivy kpvgsnepme
2161 afvgemtsyt lhnlnpstty dvnvyaqyds glsvpltdqg ttlylnvtdl ktyqigwdtf
2221 cvkwsphraa tsyrlklspa dgtrgqeitv rgsetshcft glspdtdygv tvfvqtpnle
2281 gpgvsvkeht tvkpteapte pptpppppti ppardvckga kadivfltda swsigddnfn
2341 kvvkfifntv ggfdeispag iqvsfvqysd evksefklnt yndkalalga lqniryrggn
2401 trtgkaltfi kekvltwesg mrknvpkvlv vvtdgrsqde vkkaalviqq sgfsvfvvgv
2461 advdynelan iaskpserhv fivddfesfe kiednlitfv cetatsscpl iyldgytspg
2521 fkmleaynit eknfasvqgv slesgsfpsy sayriqknaf vnqptadlhp nglppsytii
2581 llfrllpetp sdpfaiwgit drdykpqvgv iadpssktls ffnkdtrgev qtvtfdteev
2641 ktlfygsfhk vhivvtsksv kiyidcyeii ekdikeagni ttdgyeilgk llkgerksaa
2701 fqiqsfdivc spvwtsrdrc cdipsrrdeg kcpafpnsct ctqdsvgppg ppgpaggpga
2761 kgprgergis gaigppgprg digppgpqgp pgpqgpngls ipgeggrqgm kgdagepglp
2821 grtgtpglpg ppgpmgppgd rgftgkdgam gprgppgppg spgspgvtgp sgkpgkpgdh
2881 grpgpsglkg ekgdrgdias qnmmravarq vceqlisgqm nrfnqmlnqi pndyqssrnq
2941 pgppgppgpp gsagargepg pggrpgfpgt pgmqgppger glpgekgerg tgssgprglp
3001 gppgpqgesr tgppgstgsr gppgppgrpg nsgirgppgp pgycdssqca sipyngqgyp
3061 gsg
SEQ ID No. 55
Collagen, type I, alpha 2
trm|Q7Z5S6
      1 mrsrlppala algaalllss ieaevdppsd lnfkiident vhmswakpvd pivgyritvd
    61 pttdgptkef tlsasttetl lselvpetey vvtitsydev eesvpvigql tiqtgsstkp
  121 vekkpgktei qkcsysawtd lvflvdgsws vgrnnfkyil dfiaalvsaf digeektrvg
  181 vvqyssdtrt efnlnqyyqr dellaaikki pykggntmtg daidylvknt ftesagarvg
  241 fpkvaiiitd gksqdeveip arelrnvgve vfslgikaad akelkqiast pslnhvfnva
  301 nfdaivdiqn eiisqvcsgv deqlgelvsg eevveppsnl iamevsskyv klnwnpspsp
  361 vtgykviltp mtagsrqhal svgpqtttls vrdlsadtey qisysamkgm tssepisime
  421 ktqpmkvqve csrgvdikad ivflvdgsys igianfvkvr aflevlvksf eispnrvqis
  481 lvqysrdpht eftlkkftkv ediieaintf pyrggstntg kamtyvreki fvpskgsrsn
  541 vpkvmilitd gkssdafrdp aiklrnsdve ifavgvkdav rseleaiasp paethvftve
  601 dfdafqrisf eltqsiclri eqelaaikkk ayvppkdlsf sevtsygfkt nwspagenvf
  661 syhitykeaa gddevtvvep asstsvvlss lkpetlylvn vtaeyedgfs iplageette
  721 evkgaprnlk vtdettdsfk itwtqapgry lryriiyrpv aggesrevtt ppnqrrrtle
  781 nlipdtkyev svipeyfsgp gtpltgnaat eevrgnprdl rvsdpttstm klswsgapgk
  841 vkqylvtytp vaggetqevt vrgdttntvl qglkegtqya lsvtalyasg agdalfgegt
  901 tleergspqd lvtkditdts igaywtsapg mvrgyrvswk slyddvdtge knlpedaiht
  961 mienlqpetk yrisvfatys sgegepltgd attelsqdsk tlkvdeeten tmrvtwkpap
1021 gkvvnyrvvy rphgrgkqmv akvpptvtst vlkrlqpqtt yditvlpiyk mgegklrqgs
1081 gttasrfksp rnlktsdptm ssfrvtwepa pgevkgykvt fhptgddrrl gelvvgpydn
1141 tvvleelrag ttykvnvfgm fdggessplv gqemttlsdt tvmpilssgm ecltraeadi
1201 vllvdgswsi granfrtvrs fisrivevfd igpkrvqial aqysgdprte wqlnahrdkk
1261 sllqavanlp ykggntltgm alnfirqqnf rtqagmrpra rkigvlitdg ksqddveaps
1321 kklkdegvel faigiknade velkmiatdp ddthaynvad feslsrivdd ltinlcnsvk
1381 gpgdleapsn lviserthrs frvswtppsd svdrykveyy pvsggkrqef yvsrmetstv
1441 lkdlkpetey vvnvysvved eyseplkgte ktlpvpvvsl niydvgpttm hvqwqpvgga
1501 tgyilsykpv kdteptrpke vrlgptvndm qltdlvpnte yavtvgavlh dltsepvtvr
1561 evtlplprpq dlklrdvths tmnvfwepvp gkvrkyivry ktpeedvkev evdrsetsts
1621 lkdlfsqtly tvsysavhde gesppvtaqe ttrpvpaptn lkitevtseg frgtwdhgas
1681 dvslyritwa pfgssdkmet ilngdentiv fenlnpntiy evsitaiypd esesddligs
1741 ertlpilttq apksgprnlq vynatsnslt vkwdpasgry qkyrityqps tgegneqttt
1801 iggrqnsvvl qklkpdtpyt itvsslypdg eggrmtgrgk tkpintvrnl rvydpststl
1861 nvrwdhaegn prqyklfyap aaggpeelvp ipgntnyail rnlqpdtsyt vtvvpvyteg
1921 dggrtsdtgr tlmrglarnv qvynptpnsl dvrwdpapgp vlqyrvvysp vdgtrpsesi
1981 vvpgntrmvh lerlipdtly svnlvalysd gegnpspaqg rtlprsgprn lrvfgettns
2041 lsvawdhadg pvqqyriiys ptvgdpidey ttvpgrrnnv ilqplqpdtp ykitviavye
2101 dgdgghltgn grtvgllppq nihisdewyt rfrvswdpsp spvlgykivy kpvgsnepme
2161 afvgemtsyt lhnlnpstty dvnvyaqyds glsvpltdqg ttlylnvtdl ktyqigwdtf
2221 cvkwsphraa tsyrlklspa dgtrgqeitv rgsetshcft glspdtdygv tvfvqtpnle
2281 gpgvsvkeht tvkpteapte pptpppppti ppardvckga kadivfltda swsigddnfn
2341 kvvkfifntv ggfdeispag iqvsfvqysd evksefklnt yndkalalga lqniryrggn
2401 trtgkaltfi kekvltwesg mrknvpkvlv vvtdgrsqde vkkaalviqq sgfsvfvvgv
2461 advdynelan iaskpserhv fivddfesfe kiednlitfv cetatsscpl iyldgytspg
2521 fkmleaynit eknfasvqgv slesgsfpsy sayriqknaf vnqptadlhp nglppsytii
2581 llfrllpetp sdpfaiwqit drdykpqvgv iadpssktls ffnkdtrgev qtvtfdteev
2641 ktlfygsfhk vhivvtsksv kiyidcyeii ekdikeagni ttdgyeilgk llkgerksaa
2701 fqiqsfdivc spvwtsrdrc cdipsrrdeg kcpafpnsct ctqdsvgppg ppgpaggpga
2761 kgprgergis gaigppgprg digppgpqgp pgpqgpngls ipgeqgrqgm kgdagepglp
2821 grtgtpglpg ppgpmgppgd rgftgkdgam gprgppgppg spgspgvtgp sgkpgkpgdh
2881 grpgpsglkg ekgdrgdias qnmmravarq vceqlisgqm nrfnqmlnqi pndyqssrnq
2941 pgppgppgpp gsagargepg pggrpgfpgt pgmqgppger glpgekgerg tgssgprglp
3001 gppgpqgesr tgppgstgsr gppgppgrpg nsgirgppgp pgycdssqca sipyngqgyp
3061 gsg
SEQ ID No. 56
Colony stimulating factor 1 (Macrophage)
trm|Q5VVF4
      1 mtapgaagrc ppttwlgsll llvcllasrs iteevseycs hmigsghlqs lqrlidsqme
    61 tscqitfefv dqeqlkdpvc ylkkafllvq dimedtmrfr dntpnaiaiv qlqelslrlk
  121 scftkdyeeh dkacvrtfye tplqllekvk nvfnetknll dkdwnifskn cnnsfaecss
  181 qdvvtkpdcn clypkaipss dpasysphqp lapsmapvag ltwedsegte gssllpgeqp
  241 lhtvdpgsak qrpprstcqs feppetpvvk dstiggspqp rpsvgafnpg medildsamg
  301 tnwvpeeasg easeipvpqg telspsrpgg gsmqteparp snflsasspl pasakgqqpa
  361 dvtgtalpry gpvrptgqdw nhtpqktdhp sallrdppep gsprisslrp qglsnpstls
  421 aqpqlsrshs sgsvlplgel egrrstrdrr spaepeggpa segaarplpr fnsvpltdtg
  481 herqsegsfs pqlqesvfhl lvpsvilvll avggllfyrw rrrshqepqr adspleqpeg
  541 spltqddrqv elpv
SEQ ID No. 57
EGF-containing fibulin-like extracellular matrix protein 1
spt|Q12805
      1 mlkalfltml tlalvksqdt eetitytqct dgyewdpvrq qckdidecdi vpdackggmk
    61 cvnhyggylc lpktaqiivn neqpqqetqp aegtsgattg vvaassmats gvlpgggfva
  121 saaavagpem qtgrnnfvir rnpadpqrip snpshriqca agyeqsehnv cqdidectag
  181 thncradqvc inlrgsfacq cppgyqkrge qcvdidecti ppychqrcvn tpgsfycqcs
  241 pgfqlaanny tcvdinecda snqcaqqcyn ilgsficqcn qgyelssdrl ncedidecrt
  301 ssylcqyqcv nepgkfscmc pqgyqvvrsr tcqdinecet tnecredemc wnyhggfrcy
  361 prnpcqdpyi ltpenrcvcp vsnamcrelp qsivykymsi rsdrsvpsdi fqiqattiya
  421 ntintfriks gnengefylr qtspvsamlv lvkslsgpre hivdlemltv ssigtfrtss
  481 vlrltiivgp fsf
SEQ ID No. 58
Filamin A
      1 mlkalfltml tlalvksqdt eetitytqct dgyewdpvrq qckdidecdi vpdackggmk
    61 cvnhyggylc lpktaqiivn neqpqqetqp aegtsgattg vvaassmats gvlpgggfva
  121 saaavagpem qtgrnnfvir rnpadpqrip snpshriqca agyeqsehnv cqdidectag
  181 thncradqvc inlrgsfacq cppgyqkrge qcvdidecti ppychqrcvn tpgsfycqcs
  241 pgfqlaanny tcvdinecda snqcaqqcyn ilgsficqcn qgyelssdrl ncedidecrt
  301 ssylcqyqcv nepgkfscmc pqgyqvvrsr tcqdinecet tnecredemc wnyhggfrcy
  361 prnpcqdpyi ltpenrcvcp vsnamcrelp qsivykymsi rsdrsvpsdi fqiqattiya
  421 ntintfriks gnengefylr qtspvsamlv lvkslsgpre hivdlemltv ssigtfrtss
  481 vlrltiivgp fsf
SEQ ID No. 59
Follistatin-related protein 1
spt|Q12841
      1 mwkrwlalal alvavawvra eeelrskski canvfcgagr ecavtekgep tclcieqckp
    61 hkrpvcgsng ktylnhcelh rdacltgski qvdydghcke kksyspsasp vvcyqsnrde
  121 lrrriiqwle aeiipdgwfs kgsnyseild kyfknfdngd srldsseflk fvegnetain
  181 ittypdqenn kllrglcvda lielsdenad wklsfqeflk clnpsfnppe kkcaledety
  241 adgaetevdc nrcvcacgnw vctamtcdgk nqkgaqtqte eemtryvqel qkhqetaekt
  301 krvstkei
SEQ ID No. 60
Fructose-bisphosphate aldolase
trm|Q6FI10
      1 mpyqypaltp eqkkelsdia hrivapgkgi laadestgsi akrlqsigte nteenrrfyr
    61 qllltaddry npciggvilf hetlyqkadd grpfpqviks kggvvgikvd kgvvplagtn
  121 getttqgldg lsercaqykk dgadfakwrc vlkigehtps alaimenanv laryasicqq
  181 ngivpivepe ilpdgdhdlk rcqyvtekvl aavykalsdh hiylegtllk pnmvtpghac
  241 tqkfsheeia matvtalrrt vppavtgitf lsggqseeea sinlnainkc pllkpwaltf
  301 sygralqasa lkawggkken lkaageeyvk ralanslacq gkytpsgqag aaaseslfvs
  361 nhay
SEQ ID No. 61
Glucose-6-phosphate isomerase
spt|P06744
      1 maaltrdpqf qklqqwyreh rselnlrrlf dankdrfnhf sltlntnhgh ilvdysknlv
    61 tedvmrmlvd laksrgveaa rermfngeki nytegravlh valrnrsntp ilvdgkdvmp
  121 evnkvldkmk sfcqrvrsgd wkgytgktit dvinigiggs dlgplmvtea lkpyssggpr
  181 vwyvsnidgt hiaktlaqln pesslfiias ktfttqetit naetakewfl qaakdpsava
  241 khfvalstnt tkvkefgidp qnmfefwdwv ggryslwsai glsialhvgf dnfeqllsga
  301 hwmdqhfrtt pleknapvll allgiwyinc fgcethamlp ydqylhrfaa yfqqgdmesn
  361 gkyitksgtr vdhqtgpivw gepgtngqha fyqlihqgtk mipcdflipv qtqhpirkgl
  421 hhkillanfl aqtealmrgk steearkelq aagkspedle rllphkvfeg nrptnsivft
  481 kltpfmlgal vamyehkifv qgiiwdinsf dqwgvelgkq lakkiepeld gsaqvtshda
  541 stnglinfik qqrearvq
SEQ ID No. 62
Granulins (proepithelin)
spt|P28799
      1 mwtivswval taglvagtrc pdgqfcpvac cldpggasys ccrplldkwp ttlsrhlggp
    61 cqvdahcsag hsciftvsgt ssccpfpeav acgdghhccp rgfhcsadgr scfqrsgnns
  121 vgaiqcpdsq fecpdfstcc vmvdgswgcc pmpqascced rvhccphgaf cdlvhtrqt
  181 ptgthplakk lpaqrtnrav alsssvmcpd arsrcpdgst ccelpsgkyg ccpmpnatcc
  241 sdhlhccpqd tvcdliqskc lskenattdl ltklpahtvg dvkcdmevsc pdgytccrlq
  301 sgawgccpft qavccedhih ccpagftcdt qkgtceqgph qvpwmekapa hlslpdpqal
  361 krdvpcdnvs scpssdtccq ltsgewgccp ipeavccsdh qhccpqgytc vaegqcqrgs
  421 eivaglekmp arraslshpr digcdqhtsc pvgqtccpsl ggswaccqlp havccedrqh
  481 ccpagytcnv karscekevv saqpatflar sphvgvkdve cgeghfchdn qtccrdnrqg
  541 waccpyrqgv ccadrrhccp agfrcaargt kclrreaprw daplrdpalr qll
SEQ ID No.
Heat shock protein (HSP 90-alpha 2)
trm|Q5CAQ7
      1 mppcsggdgs tppgpslrdr dcpaqsaeyp rdrldprpgs pseassppfl rsrapvnwyq
    61 ekaqvflwhl lvsgsttllc lwkqpfhvsa fpvtaslafr qsqgagqhly kdlqpfillr
  121 llmpeetqtq dqpmeeeeve tfafqaeiaq lmsliintfy snkeiflrel isnssdaldk
  181 iryesltdps kldsgrelhi nlipnkqgrt ltivdtgigm tkadlinnlg tiaksgtkaf
  241 mealqagadi smigqfgvgf ysaylvaekv tvitkhndde qyawessagg sftvrtdtge
  301 pmgrgtkvil hlkedqteyl eerrikeivk khsqfigypi tlfvekerdk evsddeaeek
  361 edkeeekeke ekesedkpei edvgsdeeee kkdgdkkkkk kikekyidqe elnktkpiwt
  421 rnpdditnee ygefyksltn dwedhlavkh fsvegqlefr allfvprrap fdlfenrkkk
  481 nniklyvrry fimdnceeli peylnfirgv vdsedlpini sremlqqski lkvirknlvk
  541 kclelftela edkenykkfy eqfskniklg ihedsqnrkk lsellryyts asgdemvslk
  601 dyctrmkenq khiyyitget kdqvansafv erlrkhglev iymiepidey cvqqlkefeg
  661 ktivsvtkeg lelpedeeek kkqeekktkf enlckimkdi lekkvekvvv snrlvtspcc
  721 ivtstygwta nmerimkaqa lrdnstmgym aakkhleinp dhsiietlrq kaeadkndks
  781 vkdlvillye tallssgfsl edpqthanri yrmiklglgi deddptaddt saavteempp
  841 legdddtsrm eevd
SEQ ID No. 64
Hypothetical protein (belongs to the actin family)
trm|Q8WVW5
      1 scargkagfa gddapravfp sivgrprhqg vmvgmgqkds yvgdeaqskr giltlkypie
    61 hgivtnwddm ekiwhhtfyn elrvapeehp vllteapinp kanrekmtqi mfetfntpam
  121 yvaigavlsl yasgrttgiv mdsgdgvtht vpiyegyalp hailrldlag rdltdylmki
  181 ltergysftt taereivrdi keklcyvald feqemataas sssleksyel pdgqvitign
  241 erfrcpealf qpsflgmesc gihettfnsi mkcdvdirkd lyantvlsgg ttmypgiadr
  301 mqkeitalap stmkikiiap perkysvwig gsilaslstf qqmwiskqey desgpsivhr
  361 kcf
SEQ ID No. 65
Insulin-like growth factor binding protein 6
spt|P24592
      1 mtphrllppl llllalllaa spggalarcp gcgqgvqagc pggcveeedg gspaegcaea
    61 egclrregqe cgvytpncap glqchppkdd eaplralllg rgrclparap avaeenpkes
  121 kpqagtarpq dvnrrdqqrn pgtsttpsqp nsagvqdtem gperrhldsv lqqlqtevyr
  181 gaqtlyvpnc dhrgfyrkrq crssqgqrrg pcwcvdrmgk slpgspdgng ssscptgssg
SEQ ID No. 66
Insulin-like growth factor binding protein 7
spt|Q16270
      1 merpslrall lgaaglllll lplssssssd tcgpcepasc pplpplgcll getrdacgcc
    61 pmcargegep cggggagrgy capgmecvks rkrrkgkaga aaggpgvsgv cvcksrypvc
  121 gsdgttypsg cqlraasqra esrgekaitq vskgtceqgp sivtppkdiw nvtgaqvyls
  181 cevigiptpv liwnkvkrgh ygvqrtellp gdrdnlaiqt rggpekhevt gwvlvsplsk
  241 edageyecha snsqgqasas akitvvdalh eipvkkgega el
SEQ ID No. 67
L-lactate dehydrogenase A chain
sp|P00338
      1 matlkdqliy nllkeeqtpq nkitvvgvga vgmacaisil mkdladelal vdviedklkg
    61 emmdlqhgsl flrtpkivsg kdynvtansk lviitagarq qegesrinlv qrnvnifkfi
  121 ipnvvkyspn ckllivsnpv diltyvawki sgfpknrvig sgcnldsarf rylmgerlgv
  181 hplschgwvl gehgdssvpv wsgmnvagvs lktlhpdlgt dkdkeqwkev hkqvvesaye
  241 viklkgytsw aiglsvadla esimknlrry hpvstmikgl ygikddvfls vpqlgqngi
  301 sdlvkvtlts eeearlkksa dtlwgigkel of
SEQ ID No. 68
Matrix metalloproteinase 1 (MMP-1)
trm|Q5TZP0
      1 mhsfppllll lfwgvvshsf patletqeqd vdlvqkylek yynlkndgmq vekrrnsgpv
    61 veklkqmqef fglkvtgkpd aetlkvmkqp rcgvpdvaqf vltegnprwe qthltyrien
  121 ytpdlpradv dhaiekafql wsnvtpltft kvsegqadim isfvrgdhrd nspfdgpggn
  181 lahafqpgpg iggdahfded erwtnnfrey nlhrvaahel ghslglshst digalmypsy
  241 tfsgdvglaq ddidgiqaiy grsqnpvqpi gpqtpkacds kltfdaitti rgevmffkdr
  301 fymrtnpfyp evelnfisvf wpqlpnglea ayefadrdev rffkgnkywa vqgqnvlhgy
  361 pkdiyssfgf prtvkhidaa lseentgkty ffvankywry deykrsmdpg ypkmiandfp
  421 gighkvdavf mkdgffyffh gtrqykfdpk tkriltlqka nswfncrkn
SEQ ID No. 69
Matrix metalloproteinase 1 preprotein variant
trm|Q53G75
      1 kgmqeffglk vtgkpdaetl kvmkgprogv pdvaqfvlte gnprweqthl tyrienytpd
    61 lpradvdhvi ekafqlwsnv tpltftkvse gqadimisfv rgdhrdnspf dgpggnlaha
  121 fqpgpgiggd ahfdederwt nnfreynlhr vaahelghsl glshstdiga lmypsytfsg
  181 dvglaqndid giqaiygrsq npvqpigpqt pkacdskltf daittirgev mffkdrfymr
  241 tnpfypevel nfisvfwpql pngleaayef adrdevrffk gnkywavqgq nvlhgypkdi
  301 yssfgfprtv khidaalsee ntgktyffva nkywrydeyk rsmdpgypkm iandfpgigh
  361 kvdavfmkdg ffyffhgtrq ykfdpktkri ltlqkanswf ncrkn
SEQ ID No. 70
Nucleophosmin
spt|P06748
      1 medsmdmdms plrpqnylfg celkadkdyh fkvdndeneh qlslrtvslg agakdelhiv
    61 eaeamnyegs pikvtlatlk msvqptvslg gfeitppvvl rlkcgsgpvh isgqhlvave
  121 edaesedeee edvkllsisg krsapgggsk vpqkkvklaa deddddddee dddedddddd
  181 fddeeaeeka pvkksirdtp aknaqksnqn gkdskpsstp rskgqesfkk gektpktpkg
  241 pssvedikak mqasiekggs lpkveakfin yvkncfrmtd qeaiqdlwqw rksl
SEQ ID No. 71
Nucleoside diphosphate kinase (NME1-NME2)
trm|Q32Q12
      1 mvllstlgiv fggegppiss cdtgtmance rtfiaikpdg vqrglvgeii krfeqkgfrl
    61 vglkfmqase dllkehyvdl kdrpffaglv kymhsgpvva mvweglnvvk tgrvmlgetn
  121 padskpgtir gdfciqvgrt manlertfia ikpdgvqrgl vgeiikrfeq kgfrlvamkf
  181 lraseehlkq hyidlkdrpf fpglvkymns gpvvamvweg lnvvktgrvm lgetnpadsk
  241 pgtirgdfci qvgrniihgs dsvksaekei slwfkpeelv dykscandwv ye
SEQ ID No. 72
OAF homolog
trm|Q86UD1
      1 mrlpgvplar palllllpll apllgtgapa elrvrvrlpd gqvteeslqa dsdadsisle
    61 lrkpdgtivs ftadfkkdvk vfralilgel ekggsqfgal cfvtqlqhne iipseamakl
  121 rqknpravrq aeevrglehl hmdvavnfsq gallsphlhn vcaeavdaiy trqedvrfwl
  181 eqgvdssvfe alpkaseqae lprcrqvgdh gkpcvcrygl slawypcmlk ychsrdrptp
  241 ykcgirscqk sysfdfyvpq rqlclwdedp ypg
SEQ ID No. 73
Peptidylproylisomerase A
trm|Q3KQW3
      1 mvnptvffdi avdgeplgry sfelfadkvp ktaenfrals tgekgfgykg scfhriipgf
    61 mcqggdftrh ngtggksiyg ekfedenfil khtgpgilsm anagpntngs qffictakte
  121 wldgkhvvfg kvkegmnive amerfgsrng ktskkitiad cggle
SEQ ID No. 74
Phosphoglycerate kinase
trm|Q5J7W1
      1 mslsnkltld kldvkgkrvv mrvdfnvpmk nnqitnnqri kaavpsikfc ldngaksvvl
    61 mshlgrpdgv pmpdkyslep vavelksllg kdvlflkdcv gpevekacan paagsville
  121 nlrfhveeeg kgkdasgnkv kaepakieaf raslsklgdv yvndafgtah rahssmvgvn
  181 lpqkaggflm kkelnyfaka lesperpfla ilggakvadk iqlinnmldk vnemiigggm
  241 aftflkvinn meigtslfde egakivkdlm skaekngvki tlpvdfvtad kfdenaktgq
  301 atvasgipag wmgldcgpes skkyaeavtr akqivwngpv gvfeweafar gtkalmdevv
  361 katsrgciti igggdtatcc akwntedkvs hvstgggasl ellegkvlpg vdalsni
SEQ ID No. 75
PKM2 protein
trm|Q8WUW7
      1 gadflvteve nggslgskkg vnlpgaavdl paysekdiqd lkfgveqdvd mvfasfirka
    61 sdvhevrkvl gekgknikii skienhegvr rfdeileasd gimvargdlg ieipaekvfl
  121 aqkmmigrcn ragkpvicat qmlesmikkp rptraegsdv anavldgadc imlsgetakg
  181 dypleavrmq hliareaeaa iyhlqlfeel rrlapitsdp teatavgave asfkccsgai
  241 ivltksgrsa hqvaryrpra piiavtrnpq targahlyrg ifpvlckdpv qeawaedvdl
  301 rvnfamnvgk argffkkgdv vivltgwrpg sgftntmrvv pvp
SEQ ID No. 76
Protein CutA
spt|O60888
      1 msggrapavl lggvasllls fvwmpallpv asrllllpry lltmasgspp tqpspasdsg
    61 sgyvpgsysa afvtcpnekv akeiaravve krlaacvnli pqitsiyewk gkieedsevl
  121 mmiktqsslv paltdfvrsv hpyevaevia lpveconfpy lqwvrqvtes vsdsitvlp
SEQ ID No. 77
Protein FAM3C
spt|Q92520
      1 mrvagaaklv vavavflltf yvisqvfeik mdaslgnlfa rsaldtaars tkpprykcgi
    61 skacpekhfa fkmasgaanv vgpkicledn vlmsgvknnv grginvalan gktgevldtk
  121 yfdmwggdva pfieflkaiq dgtivlmgty ddgatklnde arrliadlgs tsitnlgfrd
  181 nwvfcggkgi ktkspfeqhi knnkdtnkye gwpevvemeg qpqkqd
SEQ ID No. −78
ProteinS100-A9
spt|P06702
      1 mtckmsqler nietiintfh qysvklghpd tlnqgefkel vrkdlqnflk kenknekvie
    61 himedldtna dkqlsfeefi mlmarltwas hekmhegdeg pghhhkpglg egtp
SEQ ID No. 79
Ribosomal protein S27a
spt|P62979
      1 mqifvktltg ktitleveps dtienvkaki qdkegippdq qrlifagkql edgrtlsdyn
    61 igkestlhlv lrlrggakkr kkksyttpkk nkhkrkkvkl avlkyykvde ngkisrlrre
  121 cpsdecgagv fmashfdrhy cgkccltycf nkpedk
SEQ ID No. 80
Secretogranin 2
trm|Q53T11
      1 maeakthwlg aalsliplif lisgaeaasf qrnqllqkep dlrlenvqkf pspemirale
    61 yienlrqqah keesspdynp yqgvsvplqq kengdeshlp erdslseedw mriilealrq
  121 aenepqsapk enkpyalnse knfpmdmsdd yetqqwperk lkhmqfppmy eensrdnpfk
  181 rtneiveeqy tpqslatles vfqelgkltg pnnqkrermd eeqklytdde ddiykannia
  241 yedvvggedw npveekiesq tqeevrdske niekneqind emkrsgqlgi qeedlrkesk
  301 dqlsddvskv iaylkrlvna agsgrlqngq ngeratrlfe kpldsqsiyq lieisrnlqi
  361 ppedliemlk tgekpngsve pereldlpvd lddiseadld hpdlfqnrml sksgypktpg
  421 ragtealpdg lsvedilnll gmesaanqkt syfpnpynqe kvlprlpyga grsrsnqlpk
  481 aawiphvenr qmayenlndk dqelgeylar mlvkypeiin snqvkrvpgq gsseddlqee
  541 eqiegaikeh lnqgssqetd klapvskrfp vgppknddtp nrqywdedll mkvleylnqe
  601 kaekgrehia kramenm
SEQ ID No. 81
SPARC (Secreted protein aqdic and rich in cysteine) (Osteonectin)
spt|P09486
      1 mrawiffllc lagralaapq qealpdetev veetvaevte vsvganpvqv evgefddgae
    61 eteeevvaen pcqnhhckhg kvceldennt pmcvcqdpts cpapigefek vcsndnktfd
  121 sschffatkc tlegtkkghk lhldyigpck yippcldsel tefplrmrdw lknvlvtlye
  181 rdednnllte kqklrvkkih enekrleagd hpvellardf eknynmyifp vhwqfgqldq
  241 hpidgylsht elaplrapli pmehcttrff etcdldndky ialdewagcf gikqkdidkd
  301 lvi
SEQ ID No. 82
Stem cell growth Factor
trm|Q5U0B9
      1 mqaawplgal vvpqllgfgh gargaerewe ggwggaqeee rerealmlkh lqealglpag
    61 rgdenpagtv egkedcemee dqgeeeeeea tptpssgpsp sptpedivty ilgrlaglda
  121 glhqlhvrlh aldtrvvelt qglrqlrnaa gdtrdavgal qeaqgraere hgrlegclkg
  181 lrlghkcfll srdfeaqaaa qarctarggs laqpadrqqm ealtrylraa lapynwpvwl
  241 gvhdrraegl ylfengqrvs ffawhrsprp elgamosasp hplspdqpng gtlencvaqa
  301 sddgswwdhd cqrrlyyvce fpf
SEQ ID No. 83
Sulfhydryl oxidase 1 (Quiesqn Q6) (hQSOX)
spt|O00391
      1 mrrcnsgsgp ppsllllllw llavpganaa prsalyspsd pltllqadtv rgavlgsrsa
    61 waveffaswc ghciafaptw kalaedvkaw rpalylaald caeetnsavc rdfnipgfpt
  121 vrffkaftkn gsgavfpvag advqtlrerl idaleshhdt wppacpplep akleeidgff
  181 arnneeylal ifekggsylg revaldlsqh kgvavrrvin teanvvrkfg vtdfpscyll
  241 frngsysrvp vlmesrsfyt aylqrlsglt reaaqttvap ttankiaptv wkladrskiy
  301 madlesalhy ilrievgrfp vlegqrlval kkfvavlaky fpgrplvqnf lhsvnewlkr
  361 qkrnkipysf fktalddrke gavlakkvnw igcqgsephf rgfpcslwvl fhfltvgaar
  421 qnvdhsqeaa kakevlpair gyvhyffgcr dcashfeqma aasmhrvgsp naavlwlwss
  481 hnrvnarlag apsedpqfpk vqwpprelcs achnerldvp vwdveatlnf lkahfspsni
  541 ildfpaagsa arrdvqnvaa apelamgale lesrnstldp gkpemmkspt nttphvpaeg
  601 peasrppklh pglraapgqe ppehmaelqr neqeqplgqw hlskrdtgaa llaesraekn
  661 rlwgplevrr vgrsskqlvd ipegqleara grgrgqwlqv lgggfsyldi slcvglysls
  721 fmgllamyty fqakiralkg haghpaa
SEQ ID No. 84
Thrombospondin 2
trm|Q5RI52
      1 mvwrlvllal wvwpstqagh qdkdttfdlf sisninrkti gakqfrgpdp gvpayrfvrf
    61 dyippvnadd lskitkimrq kegffltaql kqdgksrgtl lalegpglsq rqfeivsngp
  121 adtldltywi dgtrhvvsle dvgladsqwk nvtvqvaget yslhvgcdli dsfaldepfy
  181 ehlqaeksrm yvakgsares hfrgllqnvh lvfensvedi lskkgcqqgq gaeinaisen
  241 tetlrlgphv tteyvgpsse rrpevcersc eelgnmvqel sglhvlvnql senlkrvsnd
  301 nqflweligg ppktrnmsac wqdgrffaen etwvvdsctt ctckkfktic hqitcppatc
  361 aspsfvegec cpsclhsvdg eegwspwaew tqcsvtcgsg tqqrgrscdv tsntclgpsi
  421 qtracslskc dtrirqdggw shwspwsscs vtcgvgnitr irlcnspvpq mggknckgsg
  481 retkacqgap cpidgrwspw spwsactvtc aggirertry cnspepqygg kacvgdvqer
  541 qmcnkrscpv dgclsnpcfp gaqcssfpdg swscgscpvg flgngthced ldecalvpdi
  601 cfstskvprc vntqpgfhcl pcppryrgnq pvgvgleaak tekqvcepen pckdkthnch
  661 khaeciylgh fsdpmykcec qtgyagdgli cgedsdldgw pnlnlvcatn atyhcikdnc
  721 phlpnsgqed fdkdgigdac dddddndgvt dekdncqllf nprqadydkd evgdrcdncp
  781 yvhnpaqidt dnngegdacs vdidgddvfn erdncpyvyn tdqrdtdgdg vgdhcdncpl
  841 vhnpdqtdvd ndlvgdqcdn nedidddghq nnqdncpyis nanqadhdrd gqgdacdpdd
  901 dndgvpddrd ncrlvfnpdq edldgdgrgd ickddfdndn ipdiddvcpe nnaisetdfr
  961 nfqmvpldpk gttgidpnwv irhqgkelvq tansdpgiav gfdefgsvdf sgtfyvntdr
1021 dddyagfvfg yqsssrfyvv mwkqvtqtyw edgptraygy sgvslkvvns ttgtgehlrn
1081 alwhtgntpg qvrtlwhdpr nigwkdytay rwhlthrpkt gyirvlvheg kqvmadsgpi
1141 ydqtyaggrl glfvfsqemv yfsdlkyecr di
SEQ ID No. 85
Tissue-type plasminogen activator (PLAT protein)
trm|Q6PJA5
      1 gvqgwrenlc eeregasref kgrceaimda mkrglccvll lcgavfvsps geiharfrrg
    61 arsyqvicrd ektqmiyqqh gswlrpvlrs nrveycwcns graqchsvpv kscseprcfn
  121 ggtcqqalyf sdfvcqcpeg fagkcceidt ratcyedqgi syrgtwstae sgaectnwns
  181 salaqkpysg rrpdairlgl gnhnycrnpd rdskpwcyvf kagkyssefc stpacsegns
  241 dcyfgngsay rgthsltesg asclpwnsmi ligkvytaqn psaqalglgk hnycrnpdgd
  301 akpwchvlkn rrltweycdv pscstcglrq ysqpqfrikg glfadiashp wqaaifakhr
  361 rspgerflcg gilisscwil saahcfgerf pphhltvilg rtyrvvpgee eqkfevekyi
  421 vhkefdddty dndiallqlk sdssrcages svvrtvclpp adlqlpdwte celsgygkhe
  481 alspfyserl keahvrlyps srctsqhlln rtvtdnmlca gdtrsggpqa nlhdacqgds
  541 ggplvclndg rmtivgiisw glgcgqkdvp gvytkvtnyl dwirdnmrp
SEQ ID No. 86
Transforming growth factor, beta-induced, 68kDa variant
trm|Q53GU8
      1 malfvrllal alalalgpaa tlagpakspy qlvlqhsrlr grqhgpnvca vqkvigtnrk
    61 yftnckqwyq rkicgkstvi syeccpgyek vpgekgcpaa lplsnlyetl gvvgstttql
  121 ytdrteklrp emegpgsfti fapsneawas lpaevldslv snvniellna lryhmvgrry
  181 ltdelkhgmt ltsmyqnsni qihhypngiv tvncarllka dhhatngvvh lidkvistit
  241 nniqqiieie dtfetlraav aasglntmle gngqytllap tneafekips etlnrilgdp
  301 ealrdllnnh ilksamcaea ivaglsvetl egttlevgcs gdmltingka iisnkdilat
  361 ngvihyidel lipdsaktlf elaaesdvst aidlfrgagl gnhlsggerl tllapinsvf
  421 kdgtppidah trnllrnhii kdqlaskyly hgqtletlgg kklrvfvyrn slcienscia
  481 andkrgrygt lftmdrvltp pmgtvmdvlk gdnrfsmlva aiqsagltet lnregvytvf
  541 aptneafral pprersrllg dakelanilk yhigdeilvs ggigalvrlk slqgdklevs
  601 lknnvvsvnk epvaepdima tngvvhvitn vlqppanrpq ergdeladsa leifkqasaf
  661 srasqrsvrl apvyqkller mkh
SEQ ID No. 87
Transketolase (TK)
trm|Q53EM5
      1 mesyhkpdgq klqalkdtan rlrissiqat taagsghpts ccsaaeimav lffhtmryks
    61 gdprnphndr fvlskghaap ilyavwaeag flaeaellnl rkissdldgh pvpkgaftdv
  121 atgslgqglg aacgmaytgk yfdkasyrvy cllgdgelse gsvweamafa siykldnlva
  181 ildinrlgqs dpaplqhqmd iyqkrceafg whaiivdghs veelckafgq akhqptaiia
  241 ktfkgrgitg vedkeswhgk plpknmaegi iqeiysqiqs kkkilatppq edapsvdian
  301 irmpslpsyk vgdkiatrka yggalaklgh asdriialdg dtknstfsei fkkehpdrfi
  361 ecyiaernmv siavgcatrn rtvpfcstfa afftrafdqi rmaaisesni nlogshogvs
  421 igedgpsqma ledlamfrsv ptstvfypsd gvatekavel aantkgicfi rtsrpenaii
  481 ynnnedfqvg qakvvlkskd dqvtvigagv tlhealaaae llkkekinir vldpftikpl
  541 drklildsar atkgriltve dhyyeggige ayssavvgep gitvthlavn rvprsgkpae
  601 llkmfgidrd aiaqavrgli tka
SEQ ID No. 88
Translation elongation factor 1 alpha 1-like 14
trm|Q96RE1
      1 mqsergitid islwkfetsk yyvtiidapg hrdfiknmit gtsqadcavlivaagvgefe
    61 agiskngqtr ehallaytlg vkqlivgvnk mdsteppysq kryeeivkev styikkigyn
  121 pdtvafvpis gwngdnmlep sanmpwfkgw kvtrkdgnas gttllealdc ilpptrptdk
  181 plrlplqdvy kiggigtvpv grvetgvlkp gmvvtfapvn vttevksvem hhealsealp
  241 gdnvgfnvkn vsvkdvrrgn vagdskndpp meaagftaqv iilnhpgqis agyapvldch
  301 tahiackfae lkekidrrsg kkledgpkfl ksgdaaivdm vpgkpmcves fsdypplgrf
  361 avrdmrqtva vgvikavdkk aagagkvtks aqkaqkak
SEQ ID No. 89
Triosephosphate isomerase
trm|Q6FHP9
      1 mapsrkffvg gnwkmngrkq slgeligtln aakvpadtev vcapptayid farqkldpki
    61 avaaqncykv tngaftgeis pgmikdcgat wvvlghserr hvfgesdeli gqkvahalae
  121 glgviacige kldereagit ekvvfeqtkv iadnvkdwsk vvlayepvwa igtgktatpq
  181 qaqevheklr gwlksnvsda vaqstriiyg gsvtgatcke lasqpdvdgf lvggaslkpe
  241 fvdiinakq
SEQ ID No. 90
UV exqsion repair protein RAD23 homolog B
spt|P54727
      1 mqvtlktlqq qtfkididpe etvkalkeki esekgkdafp vagqkliyag kilnddtalk
    61 eykideknfv vvmvtkpkav stpapattqq sapasttavt ssttttvaqa ptpvpalapt
  121 stpasitpas atassepapa saakqekpae kpaetpvats ptatdstsgd ssrsnlfeda
  181 tsalvtgqsy enmvteimsm gyereqviaa lrasfnnpdr aveyllmgip gdresqavvd
  241 ppqaastgap qssavaaaaa tttattttts sgghpleflr nqpqfqqmrq iiqqnpsllp
  301 allqqigren pqllqqisqh qehfiqmlne pvqeaggqgg gggggsggia eagsghmnyi
  361 qvtpqekeai erlkalgfpe glviqayfac eknenlaanf llqqnfded
SEQ ID No. 91
Vacuolar proton pump subunit 51
spt|Q15904
      1 mmaamatary rmgprcaqal wrmpwlpvfl slaaaaaaaa aeqqvplvlw ssdrdlwapa
    61 adtheghits dlqlstyldp alelgprnvl lflqdklsie dftayggvfg nkqdsafsnl
  121 enaldlapss lvlpavdwya vstlttylqe klgasplhvd latlrelkln aslpalllir
  181 lpytassglm aprevltgnd evigqvlstl ksedvpytaa ltavrpsrva rdvavvaggl
  241 grqllqkqpv spvihppvsy ndtaprilfw aqnfsvaykd qwedltpltf gvqelnitgs
  301 fwndsfarls ltyerlfgtt vtfkfilanr lypvsarhwf tmerlevhsn gsvayfnasq
  361 vtgpsiysfh ceyvsslskk gsllvartqp spwqmmlqdf qiqafnvmge qfsyasdcas
  421 ffspgiwmgl ltslfmlfif tyglhmilsl ktmdrfddhk gptisltqiv
SEQ ID NO. 92
Serum thyroglobulin
spt|P01266
      1 malvleiftl lasicwvsan ifeyqvdaqp lrpcelqret aflkqadyvp qcaedgsfqt
    61 vqcqndgrsc wcvgangsev lgsrqpgrpv aclsfcqlqk qqillsgyin stdtsylpqc
  121 qdsgdyapvq cdvqqvqcwc vdaegmevyg trqlgrpkrc prsceirnrr llhgvgdksp
  181 pqcsaegefm pvqckfvntt dmmifdlvhs ynrfpdafvt fssfqrrfpe vsgychcads
  241 qgrelaetgl ellldeiydt ifagldlpst ftettlyril qrrflavqsv isgrfrcptk
  301 ceverftats fghpyvpscr rngdyqavqc qtegpcwcvd aqgkemhgtr qqgeppscae
  361 gqscaserqq alsrlyfgts gyfsqhdlfs spekrwaspr varfatscpp tikelfvdsg
  421 llrpmvegqs qqfsysenll keairaifps rglarlalqf ttnpkrlqqn lfggkflvnv
  481 gqfnlsgalg trgtfnfsqf fqqlglasfl nggrqedlak plsvgldsns stgtpeaakk
  541 dgtmnkptvg sfgfeinlqe nqnalkflas llelpefllf lqhaisvped vardlgdvme
  601 tvlssqtceq tperlfvpsc ttegsyedvq cfsgecwcvn swgkelpgsr vrggqprcpt
  661 dcekqrarmq slmgsqpags tlfvpactse ghflpvqcfn secycvdaeg qaipgtrsai
  721 gkpkkcptpc qlqseqaflr tvqallsnss mlptlsdtyi pqcstdgqwr qvqcngppeq
  781 vfelyqrwea qnkgqdltpa kllvkimsyr eaasgnfslf iqslyeagqq dvfpvlsqyp
  841 slqdvplaal egkrpqpren illepylfwq ilngqlsqyp gsysdfstpl ahfdlrncwc
  901 vdeaggeleg mrsepsklpt cpgsceeakl rvlqfirete eivsasnssr fplgesflva
  961 kgirlrnedl glpplfppre afaeqflrgs dyairlaaqs tlsfyqrrrf spddsagasa
1021 llrsgpympq cdafgswepv qchagtghcw cvdekggfip gsltarslqi pqcpttceks
1081 rtsgllsswk qarsqenpsp kdlfvpacle tgeyarlqas gagtwcvdpa sgeelrpgss
1141 ssaqcpslcn vlksgvlsrr vspgyvpacr aedggfspvq cdqaqgscwc vmdsgeevpg
1201 trvtggqpac esprcplpfn asevvggtil cetisgptgs amqqcqllcr qgswsvfppg
1261 plicslesgr wesqlpqpra cqrpqlwqti qtqghfqlql ppgkmcsady adllqtfqvf
1321 ildeltargf cqiqvktfgt lvsipvcnns svqvgcltre rlgvnvtwks rledipvasl
1381 pdlhdieral vgkdllgrft dliqsgsfql hldsktfpae tirflqgdhf gtsprtwfgc
1441 segfyqvlts easqdglgcv kcpegsysqd eecipcpvgf yqeqagslac vpcpvgrtti
1501 sagafsqthc vtdcqrneag lqcdqngqyr asqkdrgsgk afcvdgegrr lpwweteapl
1561 edsqclmmqk fekvpeskvi fdanapvavr skvpdsefpv mqcltdcted eacsfftvst
1621 tepeiscdfy awtsdnvacm tsdqkrdalg nskatsfgsl rcqvkvrshg qdspavylkk
1681 gqgstttlqk rfeptgfqnm lsglynpivf sasganitda hlfcllacdr dlccdgfvlt
1741 qvqggaiicg llsspsvllc nvkdwmdpse awanatcpgv tydqeshqvi lrlgdgefik
1801 sltplegtqd tftnfqqvyl wkdsdmgsrp esmgcrkdtv prpaspteag lttelfspvd
1861 lnqvivngnq slssqkhwlf khlfsaqqan lwolsrcvqe hsfcglaeit esaslyftct
1921 lypeaqvcdd imesnaggcr lilpqmpkal frkkviledk vknfytrlpf qklmgisirn
1981 kvpmseksis ngffecerrc dadpcctgfg flnvsqlkgg evtcltlnsl giqmcseeng
2041 gawrildcgs pdievhtypf gwyqkpiagn napsfcplvv lpsltekvsl dswqslalss
2101 vvvdpsirhf dvahvstaat snfsavrdlc lsecsgheac littlqtqpg avrcmfyadt
2161 qscthslqgq ncrlllreea thiyrkpgis llsyeasvps vpisthgrll grsgaiqvgt
2221 swkqvdqflg vpyaapplae rrfqapepin wtgswdaskp rascwqpgtr tstspgvsed
2281 clylnvfipq nvapnasvlv ffhntmdree segwpaidgs flaavgnliv vtasyrvgvf
2341 gflssgsgev sgnwglldqv aaltwvqthi rgfggdprry slaadrggad vasihlltar
2401 atnsqlfrra vlmggsalsp aavisheraq qqaialakev scpmsssgev vsclrqkpan
2461 vindaqtkll aysgpfhywg pvidghflre pparalkrsl wvevdlligs sqddglinra
2521 kavkgfeesr grtssktafy galqnslgge dsdarveaaa twyyslehst ddyasfsral
2581 enatrdyfii cpiidmasaw akrargnvfm yhapenyghg slelladvqf alglpfypay
2641 eggfsleeks lslkimgyfs hfirsgnpny pyefsrkvpt fatpwpdfvp raggenykef
2701 sellpnrqgl kkadcsfwsk yisslktsad gakggqsaes eeeeltagsg lredllslge
2761 pgsktysk
SEQ ID No. 93
BRAF mutation
spt|P15056
      1 maalsggggg gaepgqalfn gdmepeagag agaaassaad paipeevwni kqmikltgeh
    61 iealldkfgg ehnppsiyle ayeeytskld alggreqqll eslgngtdfs vsssasmdtv
  121 tsssssslsv lpsslsvfqn ptdvarsnpk spqkpivrvf lpnkgrtvvp arcgvtvrds
  181 lkkalmmrgl ipeccavyri qdgekkpigw dtdiswltge elhvevlenv pltthnfvrk
  241 tfftlafcdf crkllfqgfr cqtcgykfhq rcstevplmc vnydqldllf vskffehhpi
  301 pgeeaslaet altsgsspsa pasdsigpqi ltspspsksi pipqpfrpad edhrnqfgqr
  361 drsssapnvh intiepvnid dlirdqgfrg dggsttglsa tppaslpgsl tnvkalqksp
  421 gpqrerksss ssedrnrmkt lgrrdssddw eipdgqitvg grigsgsfgt vykgkwhgdv
  481 avkmlnvtap tpqqlqafkn evgvlrktrh vnillfmgys tkpglaivtg wcegsslyhh
  541 lhiietkfem iklidiarqt acomdylhak siihrdlksn niflhedltv kigdfglatv
  601 ksrwsgshqf eqlsgsilwm apevirmqdk npysfqsdvy afgivlyelm tgqlpysnin
  661 nrdqiifmvg rgylspdlsk vrsncpkamk rlmaeclkkk rderplfpqi lasiellars
  721 lpkihrsase pslnragfqt edfslyacas pktpigaggy gafpvh
SEQ ID No. 94
Vimentin
trm|Q5JVTO
      1 mstrsyssss yrrmfggpgt asrpsssrsy vttstrtysl gsalrpstsr slyasspggv
    61 yatrssavrl rssvpgvrll qdsvdfslad aintefkntr tnekvelgel ndrfanyidk
  121 vrfleqqnki llaeleglkg qgksrlgdly eeemrelrrq vdqltndkar veverdnlae
  181 dimrlreklq eemlgreeae ntlgsfrqdv dnaslarldl erkveslgee iaflkklhee
  241 eigelgagiq eqhvgidvdv skpdltaalr dvrqqyesva aknlqeaeew ykskfadlse
  301 aanrnndalr qakqesteyr rqvgsltcev dalkgtnesl erqmremeen faveaanyqd
  361 tigrlqdeig nmkeemarhl reyqdllnvk maldieiaty rkllegeesr islplpnfss
  421 lnlrgkhfis l
SEQ ID No. −95
Galectin-1
spt|P09382
      1 macglvasnl nlkpgeclry rgevapdaks fvinlgkdsn nlclhfnprf nahgdantiv
    61 cnskdggawg tegreavfpf qpgsvaevq tfolganitvk lpdgyefkfp nrinleainy
  121 maadgdfkik cvafd
```

What is claimed is:

1. A method for diagnosing thyroid cancer in a subject comprising:
detecting in a sample from the subject an amount of one or more Thyroid Cancer Markers as set out in Table 1, the one or more Thyroid Cancer Markers comprising activated leukocyte cell adhesion molecule (ALCAM);
comparing the detected amount of ALCAM with a predetermined standard, wherein the predetermined standard is:
(a) a first predetermined standard comprising: an amount of ALCAM in benign tissue; an amount of ALCAM in non-cancerous tissue; or an amount of ALCAM in tissue from the subject obtained at an earlier time; or,
(b) a second predetermined standard comprising an amount of ALCAM in cancerous tissue;
and diagnosing the presence of thyroid cancer when the detected amount of ALCAM in the sample is less than the first predetermined standard or greater than the second predetermined standard.

2. The method of claim 1, wherein ALCAM is detected by the following steps:
contacting the sample with an antibody that specifically binds to ALCAM or a part thereof; and
measuring in the sample an amount of ALCAM bound to the antibody.

3. The method as claimed in claim 1, wherein the thyroid cancer is aggressive or metastatic thyroid cancer and the one or more Thyroid Cancer Markers are chosen from activated leukocyte cell adhesion molecule (ALCAM)/CD166, tyrosine-protein kinase receptor UFO (AXL), amyloid precursor protein like protein 2 (APLP2), amyloid precursor protein (APP), biotinidase, cadherin-2, prothymosin-alpha, clusterin, syndecan-4, E-cadherin, gelsolin, hnRNP A2/B1, nucleolin, pyruvate kinase M2, α-enolase, 14-3-3 zeta, α-MCFD2, α-NPC2, calsyntenin and SET protein.

4. The method as claimed in claim 1, wherein the thyroid cancer is papillary thyroid cancer and the one or more Thyroid Cancer Markers are chosen from gamma-glutamyl hydrolase, lysyl oxidase-like 2, biotinidase and nidogen-1, and optionally CYR61 and/or E-cadherin.

5. The method as claimed in claim 1, wherein the thyroid cancer is follicular thyroid cancer or papillary thyroid cancer.

6. The method of claim 1, wherein the sample is a biological fluid.

7. The method of claim 6, wherein the sample is serum.

8. A method for diagnosing thyroid cancer in a subject comprising:
contacting a sample from the subject with antibodies that bind to one or more Thyroid Cancer Markers as set out in Table 1, wherein the antibodies comprise at least one antibody that specifically binds to activated leukocyte cell adhesion molecule (ALCAM) or a part thereof;
detecting in the sample from the subject an amount of ALCAM bound to the at least one antibody;
comparing the detected amount of bound ALCAM with a predetermined standard, wherein the predetermined standard is:
(a) a first predetermined standard comprising: an amount of ALCAM in benign tissue; an amount of ALCAM in non-cancerous tissue; or an amount of ALCAM in tissue from the subject obtained at an earlier time; or,
(b) a second predetermined standard comprising an amount of ALCAM in cancerous tissue;
and diagnosing the presence of thyroid cancer when the detected amount of ALCAM in the sample is less than the first predetermined standard or greater than the second predetermined standard.

9. The method of claim 1, wherein the sample is a thyroid tissue sample.

10. The method of claim 1, wherein the cancer is aggressive or non-aggressive thyroid cancer.

11. The method of claim 8, wherein the sample is a thyroid tissue sample.

12. The method of claim 8, wherein the cancer is aggressive or non-aggressive thyroid cancer.

13. The method of claim 8, wherein the thyroid cancer is follicular thyroid cancer or papillary thyroid cancer.

14. The method of claim 8, wherein the sample is a biological fluid.

15. The method of claim 8, wherein the sample is serum.


SEQUENCE LISTING (95 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRRGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 3 - protein, 105 aa, Homo sapiens
MSDAAVDTSSEITTEDLKEKKEVVEEAENGRDAPAHGNANEENGEPEADNEVDEEEEEGGEEEGDGEEEDGDEDEGAESATGKRAAEDDEDDDVDTQKQKTDEDD

SEQ ID NO: 4 - protein, 151 aa, Homo sapiens
MRFLAATFLLLALSTAAQAEPVQFRDCGSVDGVIKEVNVSPCPTQPCQLSKGQSYSVNVTFTSNVQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 5 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 6 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 7 - protein, 522 aa, Homo sapiens [sequence omitted]

SEQ ID NO: 8 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 9 - protein, 49 aa, Homo sapiens
VFFAEDVGSNKGAIIGLMVGGVVIATVIFITLVMLKKKQYTSIHHGVVE

SEQ ID NO: 10 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 11 - protein, 277 aa, Homo sapiens
MSAQAAKVSKKELNSNHDGADETSEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 12 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKRKGADFLVTEVENGGSLGSKKGVNLPGAAVDLPAVSEKDIQDLKFGVEQDVDMVFASFIRKASDVREVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 13 - protein, 379 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSII

SEQ ID NO: 14 - protein, 971 aa, Homo sapiens
MLRRPAPALAPAARLLLAGLLCGGGVWAARVNKHKPWLEPTYHGIVTENDNTVLLDPPLIALDKDAPLRFAGEICGFKIHGQNVPFDAVVVDKSTGEGVIRSKEKLDCELQKDYSFTIQAYDCGKGPDGTNVKKSHKATVHIQVNDVNEYAPVFKEKSYKATVIEGKQYDSILRVEAVDADCSPQFSQICSYEIITPDVPFTVDKDGYIKNTEKLNYGKEHQYKLTVTAYDCGKKRATEDVLVKISIKPTCTPGWQGWNNRIEYEPGTGALAVFPNIHLETCDEPVASVQATVELETSHIGKGCDRDTYSEKSLHRLCGAAAGTAELLPSPSGSLNWTMGLPTDNGHDSDQVFEFNGTQAVRIPDGVVSVSPKEPFTISVWMRHGPFGRKKETILCSSDKTDMNRHHYSLYVHGCRLIFLFRQDPSEEKKYRPAEFHWKLNQVCDEEWHHYVLNVEFPSVTLYVDGTSHEPFSVTEDYPLHPSKIETQLVVGACWQEFSGVENDNETEPVTVASAGGDLHMTQFFRGNLAGLTLRSGKLADKKVIDCLYTCKEGLDLQVLEDSGRGVQIQAHPSQLVLTLEGEDLGELDKAMQHISYLNSRQFPTPGIRRLKITSTIKCFNEATCISVPPVDGYVMVLQPEEPKISLSGVHHFARAASEFESSEGVFLFPELRIISTITREVEPEGDGAEDPTVQESLVSEEIVHDLDTCEVTVEGEELNHEQESLEVDMARLQQKGIEVSSSELGMTFTGVDTMASYEEVLHLLRYRNWHARSLLDRKFKLICSELNGRYISNEFKVEVNVIHTANPMEHANHMAAQPQFVHPEHRSFVDLSGHNLANPHPFAVVPSTATVVIVVCVSFLVFMIILGVFRIRAAHRRTMRDQDTGKENEMDWDDSALTITVNPMETYEDQHSSEEEEEEEEEEESEDGEEEDDITSAESESSEEEEGEQGDPQNATRQQQLEWDDSTLSY

SEQ ID NO: 15 - protein, 350 aa, Homo sapiens [sequence omitted]

SEQ ID NO: 16 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 17 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 18 - protein, 461 aa, Homo sapiens
MPPSGPRGTLLLLPLLLLLLLRAVLAVPLERGAPNKEETPATESPDTGLYYHRYLQEVIDVLETDGHFREKLQAANAEDIKSGKLSRELDFVSHHVRTRLDELKRQEVSRLRMLLKAKMDAEQDPNVQVDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWETVEMHPAYTEEELRRFEEELAAREAELNAKAQRLSQETEALGRSQGRLEAQKRELQQAVLHMEQRKQQQQQQQGHKAPAAHPEGQLKFHPDTDDVPVPAPAGDQKEVDTSEKKLLERLPEVEVPQHL

SEQ ID NO: 19 - protein, 381 aa, Homo sapiens
MSSRIARALALVVTLLHLTRLALSTCPAACHCPLEAPKCAPGVGLVRDGCGCCKVCAKQLNEDCSKTQPCDHTKGLECNFGASSTALKGICRAQSEGRPCEYNSRIYQNGESFQPSCKHQCTCIDGAVGCIPLCPQELSLPNLGCPNPRLVKVTGQCCEEWVCDEDSIKDPMEDQDGLLGKELGFDASEVELTRNNELIAVGKGSSLKRLPVFGMEPRILYNPLQGQKCIVQTTSWSQCSKTCGTGISTRVTNDNPECRLVKETRICEVRPCGQPVYSSLKKGKKCSKTKKSPEPVRFTYAGCLSVKKYRPKYCGSRVDGRCCTPQLTRTVKMRFRCEDGETFSKNVMMIQSCKCNYNCPHANEAAFPFYRLFNDIHKFRG

SEQ ID NO: 20 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 21 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKERYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 22 - protein, 895 aa, Homo sapiens
MRMSVGLSLLLPLWGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 23 - protein, 1479 aa, Homo sapiens
MAKRSRGPGRRCLLALVLFCAWGTLAVVAQKPGAGCPSRCLCFRTTVRCMHLLLEAVPAVAPQTSILDLRFNRIREIQPGAFRRLRNLNTLLLNNNQIKRIPSGAFEDLENLKYLYLYKNEIQSIDRQAFKGLASLEQLYLHFNQIETLDPDSFQHLPKLERLFLHNNRITHLVPGTFNHLESMKRLRLDSNTLHCDCEILWLADLLKTYAESGNAQAAAICEYPRRIQGRSVATITPEELNCERPRITSEPQDADVTSGNTVYFTCRAEGNPKPEIIWLRNNNELSMKTDSRLNLLDDGTLMIQNTQETDQGIYQCMAKNVAGEVKTQEVTLRYFGSPARPTFVIQPQNTEVLVGESVTLECSATGHPPPRISWTRGDRTPLPVDPRVNITPSGGLYIQNVVQGDSGEYACSATNNIDSVHATAFIIVQALPQFTVTPQDRVVIEGQTVDFQCEAKGNPPPVIAWTKGGSQLSVDRRHLVLSSGTLRISGVALHDQGQYECQAVNIIGSQKVVAHLTVQPRVTPVFASIPSDTTVEVGANVQLPCSSQGEPEPAITWNKDGVQVTESGKFHISPEGFLTINDVGPADAGRYECVARNTIGSASVSMVLSVNVPDVSRNGDPFVATSIVEAIATVDRAINSTRTHLFDSRPRSPNDLLALFRYPRDPYTVEQARAGEIFERTLQLIQEHVQHGLMVDLNGTSYHYNDLVSPQYLNLIANLSGCTAHRRVNNCSDMCFHQKYRTHDGTCNNLQHPMWGASLTAFERLLKSVYENGFNTPRGINPHRLYNGHALPMPRLVSTTLIGTETVTPDEQFTHMLMQWGQFLDHDLDSTVVALSQARFSDGQHCSNVCSNDPPCFSVMIPPNDSRARSGARCMFFVRSSPVCGSGMTSLLMNSVYPREQINQLTSYIDASNVYGSTEHEARSIRDLASHRGLLRQGIVQRSGKPLLPFATGPPTECMRDENESPIPCFLAGDHRANEQLGLTSMHTLWFREHNRIATELLKLNPHWDGDTIYYETRKIVGAEIQHITYQHWLPKILGEVGMRTLGEYHGYDPGINAGIFNAFATAAFRFGHTLVNPLLYRLDENFQPIAQDHLPLHKAFFSPFRIVNEGGIDPLLRGLFGVAGKMRVPSQLLNTELTERLFSMAHTVALDLAAINIQRGRDHGIPPYHDYRVYCNLSAAHTFEDLKNEIKNPEIREKLKRLYGSTLNIDLFPALVVEDLVPGSRLGPTLMCLLSTQFKRLRDGDRLWYENPGVFSPAQLTQIKQTSLARILCDNADNITRVQSDVFRVAEFPHGYGSCDEIPRVDLRVWQDCCEDCRTRGQFNAFSYHFRGRRSLEFSYQEDKPTKKTRPRKIPSVGRQGEHLSNSTSAFSTRSDASGTNDFREFVLEMQKTITDLRTQIKKLESRLSTTECVDAGGESHANNTKWKKDACTICECKDGQVTCFVEACPPATCAVPVNIPGACCPVCLQKRAEEKP

SEQ ID NO: 24 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 25 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 26 - protein, 149 aa, Homo sapiens [sequence omitted]

SEQ ID NO: 27 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 28 - protein, 2444 aa, Homo sapiens [sequence omitted]

SEQ ID NO: 29 - protein, 128 aa, Homo sapiens
MQIFVKTLTGKTITLEVEPSDTIENVKAKIQDKEGIPPDQQRLIFAGKQLEDGRTLSDYNIQKESTLHLVLRLRGGIIEPSLRQLAQKYNCDKMICRKCYARLHPRAVNCRKKKCGHTNNLRPKKKVK

SEQ ID NO: 30 - protein, 4391 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS

SEQ ID NO: 31 - protein, 3410 aa, Homo sapiens
IFPPNCNKPPSKAKMFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVLVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 32 - protein, 318 aa, Homo sapiens
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 33 - protein, 774 aa, Homo sapiens
MERPLCSHLCSCLAMLALLSPLSLAQYDSWPHYPEYFQQPAPEYHQPQAPANVAKIQLRLAGQKRKHSEGRVEVYYDGQWGTVCDDDFSIHAAHVVCRELGYVEAKSWTASSSYGKGEGPIWLDNLHCTGNEATLAACTSNGWGVTDCKHTEDVGVVCSDKRIPGFKFDNSLINQIENLNIQVEDIRIRAILSTYRKRTPVMEGYVEVKEGKTWKQICDKHWTAKNSRVVCGMFGFPGERTYNTKVYKMFASRRKQRYWPFSMDCTGTEAHISSCKLGPQVSLDPMKNVTCENGLPAVVSCVPGQVFSPDGPSRFRKAYKPEQPLVRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACSETAPDLVLNAEMVQQTTYLEDRPMFMLQCAMEENCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITMGCWDMYRHDIDCQWVDITDVPPGDYLFQVVINPNFEVAESDYSNNIMKCRSRYDGHRIWMYNCHIGGSFSEETEKKFEHFSGLLNNQLSPQ

SEQ ID NO: 34 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 35 - protein, 353 aa, Homo sapiens
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 36 - protein, 94 aa, Homo sapiens
MEHLEGVINKPEAEMSPQELQLHYFKMHDYDGNNLLDGLELSTAITHVHKEEGSEQAPLMSEDELINIIDGVLRDDDKNNDGYIDYAEFAKSLQ

SEQ ID NO: 37 - protein, 151 aa, Homo sapiens; identical to SEQ ID NO: 4

SEQ ID NO: 38 - protein, 255 aa, Homo sapiens; identical to SEQ ID NO: 5

SEQ ID NO: 39 - protein, 115 aa, Homo sapiens
MRYVASYLLAALGGNSSPSAKDIKKILDSVGIEADDDRLNKVISELNGKNIEDVIAQGIGKLASVPAGGAVAVSAAPGSAAPAAGSAPAAAEEKKDEKKEESEESDDDMGFGLFD

SEQ ID NO: 40 - protein, 2045 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 41 - protein, 892 aa, Homo sapiens
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRFAIQDISVEETSAKEGLLLWCQRKTAPYKNVNIQNFHISWKDGLGFCALIHRHRPELIDYGKLRKDDPLTNLNTAFDVAEKYLDIPKMLDAEDIVGTARPDEKAIMTYVSSFYHAFSGAQKAETAANRICKVLAVNQENEQLMEDYEKLASDLLEWIRRTIPWLENRVPENTMHAMQQKLEDFRDYRRLHKPPKVQEKCQLEINFNTLQTKLRLSNRPAFMPSEGRMVSDINNAWGCLEQVEKGYEEWLLNEIRRLERLDHLAEKFRQKASIHEAWTDGKEAMLRQKDYETATLSEIKALLKKHEAFESDLAAHQDRVEQIAAIAQELNELDYYDSPSVNARCQKICDQWDNLGALTQKRREALERTEKLLETIDQLYLEYAKRAAPFNNWMEGAMEDLQDTFIVHTIEEIQGLTTAHEQFKATLPDADKERLAILGIHNEVSKIVQTYHVNMAGTNPYTTITPQEINGKWDHVRQLVPRRDQALTEEHARQQHNERLRKQFGAQANVIGPWIQTKMEEIGRISIEMHGTLEDQLSHLRQYEKSIVNYKPKIDQLEGDHQLIQEALIFDNKHTNYTMEHIRVGWEQLLTTIARTINEVENQILTRDAKGISQEQMNEFRASFNHFDRDHSGTLGPEEFKACLISLGYDIGNDPQGEAEFARIMSIVDPNRLGVVTFQAFIDFMSRETADTDTADQVMASFKILAGDKNYITMDELRRELPPDQAEYCIARMAPYTGPDSVPGALDYMSFSTALYGESDL

SEQ ID NO: 42 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 43 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 44 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 45 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 46 - protein, 406 aa, Homo sapiens
LGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKGDDEDKDEDEEDEEDKEEDEEEDVPGQAKDEL

SEQ ID NO: 47 - protein, 463 aa, Homo sapiens [sequence omitted]

SEQ ID NO: 48 - protein, 303 aa, Homo sapiens [sequence omitted]

SEQ ID NO: 49 - protein, 603 aa, Homo sapiens
MVKLAKAGKNQGDPKKMAPPPKEVEEDSEDEEMSEDEEDDEDDDDDEEDDSEEEAMETTPAKGKKAAKVVPVKAKGAKNGKNAKKEDSDEEEDDDSEEDEEDDEDEDEDEDEIEPAAMKAAAAAPASEDEDDEDDEDDEDDDDDEDDEDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGNSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 50 - protein, 97 aa, Homo sapiens
AGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILG

SEQ ID NO: 51 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 52 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 53 - protein, 1028 aa, Homo sapiens
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGSSGPSGDEGQPGEPGPPGEKGEAGDEGNPGPDGAPGERGGPGERGPRGTPGTRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVAYGESHLFRVPSYQALLRGVFHQTVSRKVALG

SEQ ID NO: 54 - protein, 3062 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 55 - protein, 3063 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 56 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 57 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 58 - protein, 493 aa, Homo sapiens; identical to SEQ ID NO: 57

SEQ ID NO: 59 - protein, 308 aa, Homo sapiens
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 60 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 61 - protein, 558 aa, Homo sapiens
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 62 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 63 - protein, 854 aa, Homo sapiens
MPPCSGGDGSTPPGPSLRDRDCPAQSAEYPRDRLDPRPGSPSEASSPPFLRSRAPVNWYQEKAQVFLWHLLVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGRELHINLIPNKQGRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 64 - protein, 363 aa, Homo sapiens
SCARGKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 65 - protein, 240 aa, Homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 66 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 67 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 68 - protein, 469 aa, Homo sapiens [sequence omitted]

SEQ ID NO: 69 - protein, 405 aa, Homo sapiens
KQMQEFFGLKVTGKPDAETLKVMKQPRCGVPDVAQFVLTEGNPRWEQTHLTYRIENYTPDLPRADVDHVIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQNDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 70 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 71 - protein, 292 aa, Homo sapiens
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRTMANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 72 - protein, 273 aa, Homo sapiens
MRLPGVPLARPALLLLLPLLAPLLGTGAPAELRVRVRLPDGQVTEESLQADSDADSISLELRKPDGTLVSFTADFKKDVKVFRALILGELEKGQSQFQALCFVTQLQHNEIIPSEAMAKLRQKNPRAVRQAEEVRGLEHLHMDVAVNFSQGALLSPHLHNVCAEAVDAIYTRQEDVRFWLEQGVDSSVFEALPKASEQAELPRCRQVGDHGKPCVCRYGLSLAWYPCMLKYCHSRDRPTPYKCGIRSCQKSYSFDFYVPQRQLCLWDEDPYPG

SEQ ID NO: 73 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 74 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 75 - protein, 343 aa, Homo sapiens
GADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 76 - protein, 179 aa, Homo sapiens
MSGGRAPAVLLGGVASLLLSFVWMPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 77 - protein, 227 aa, Homo sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 78 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 79 - protein, 156 aa, Homo sapiens
MQIFVKTLTGKTITLEVEPSDTIENVKAKIQDKEGIPPDQQRLIFAGKQLEDGRTLSDYNIQKESTLHLVLRLRGGAKKRKKKSYTTPKKNKHKRKKVKLAVLKYYKVDENGKISRLRRECPSDECGAGVFMASHFDRHYCGKCCLTYCFNKPEDK

SEQ ID NO: 80 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 81 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 82 - protein, 323 aa, Homo sapiens [sequence omitted]

SEQ ID NO: 83 - protein, 747 aa, Homo sapiens
MRRCNSGSGPPPSLLLLLLWLLAVPGANAAPRSALYSPSDPLTLLQADTVRGAVLGSRSAWAVEFFASWCGHCIAFAPTWKALAEDVKAWRPALYLAALDCAEETNSAVCRDFNIPGFPTVRFFKAFTKNGSGAVFPVAGADVQTLRERLIDALESHHDTWPPACPPLEPAKLEEIDGFFARNNEEYLALIFEKGGSYLGREVALDLSQHKGVAVRRVLNTEANVVRKFGVTDFPSCYLLFRNGSVSRVPVLMESRSFYTAYLQRLSGLTREAAQTTVAPTTANKIAPTVWKLADRSKIYMADLESALHYILRIEVGRFPVLEGQRLVALKKFVAVLAKYFPGRPLVQNFLHSVNEWLKRQKRNKIPYSFFKTALDDRKEGAVLAKKVNWIGCQGSEPHFRGFPCSLWVLFHFLTVQAARQNVDHSQEAAKAKEVLPAIRGYVHYFFGCRDCASHFEQMAAASMHRVGSPNAAVLWLWSSHNRVNARLAGAPSEDPQFPKVQWPPRELCSACHNERLDVPVWDVEATLNFLKAHFSPSNIILDFPAAGSAARRDVQNVAAAPELAMGALELESRNSTLDPGKPEMMKSPTNTTPHVPAEGPEASRPPKLHPGLRAAPGQEPPEHMAELQRNEQEQPLGQWHLSKRDTGAALLAESRAEKNRLWGPLEVRRVGRSSKQLVDIPEGQLEARAGRGRGQWLQVLGGGFSYLDISLCVGLYSLSFMGLLAMYTYFQAKIRALKGHAGHPAA

SEQ ID NO: 84 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 85 - protein, 589 aa, Homo sapiens
GVQGWRENLCEEREGASREFKGRCEAIMDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 86 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGGERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 87 - protein, 623 aa, Homo sapiens
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAERNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 88 - protein, 398 aa, Homo sapiens [sequence omitted]

SEQ ID NO: 89 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 90 - protein, 409 aa, Homo sapiens
MQVTLKTLQQQTFKIDIDPEETVKALKEKIESEKGKDAFPVAGQKLIYAGKILNDDTALKEYKIDEKNFVVVMVTKPKAVSTPAPATTQQSAPASTTAVTSSTTTTVAQAPTPVPALAPTSTPASITPASATASSEPAPASAAKQEKPAEKPAETPVATSPTATDSTSGDSSRSNLFEDATSALVTGQSYENMVTEIMSMGYEREQVIAALRASFNNPDRAVEYLLMGIPGDRESQAVVDPPQAASTGAPQSSAVAAAAATTTATTTTTSSGGHPLEFLRNQPQFQQMRQIIQQNPSLLPALLQQIGRENPQLLQQISQHQEHFIQMLNEPVQEAGGQGGGGGGGSGGIAEAGSGHMNYIQVTPQEKEAIERLKALGFPEGLVIQAYFACEKNENLAANFLLQQNFDED

SEQ ID NO: 91 - protein, 470 aa, Homo sapiens
MMAAMATARVRMGPRCAQALWRMPWLPVFLSLAAAAAAAAAEQQVPLVLWSSDRDLWAPAADTHEGHITSDLQLSTYLDPALELGPRNVLLFLQDKLSIEDFTAYGGVFGNKQDSAFSNLENALDLAPSSLVLPAVDWYAVSTLTTYLQEKLGASPLHVDLATLRELKLNASLPALLLIRLPYTASSGLMAPREVLTGNDEVIGQVLSTLKSEDVPYTAALTAVRPSRVARDVAVVAGGLGRQLLQKQPVSPVIHPPVSYNDTAPRILFWAQNFSVAYKDQWEDLTPLTFGVQELNLTGSFWNDSFARLSLTYERLFGTTVTFKFILANRLYPVSARHWFTMERLEVHSNGSVAYFNASQVTGPSIYSFHCEYVSSLSKKGSLLVARTQPSPWQMMLQDFQIQAFNVMGEQFSYASDCASFFSPGIWMGLLTSLFMLFIFTYGLHMILSLKTMDRFDDHKGPTISLTQIV

SEQ ID NO: 92 - protein, 2768 aa, Homo sapiens
MALVLEIFTLLASICWVSANIFEYQVDAQPLRPCELQRETAFLKQADYVPQCAEDGSFQTVQCQNDGRSCWCVGANGSEVLGSRQPGRPVACLSFCQLQKQQILLSGYINSTDTSYLPQCQDSGDYAPVQCDVQQVQCWCVDAEGMEVYGTRQLGRPKRCPRSCEIRNRRLLHGVGDKSPPQCSAEGEFMPVQCKFVNTTDMMIFDLVHSYNRFPDAFVTFSSFQRRFPEVSGYCHCADSQGRELAETGLELLLDEIYDTIFAGLDLPSTFTETTLYRILQRRFLAVQSVISGRFRCPTKCEVERFTATSFGHPYVPSCRRNGDYQAVQCQTEGPCWCVDAQGKEMHGTRQQGEPPSCAEGQSCASERQQALSRLYFGTSGYFSQHDLFSSPEKRWASPRVARFATSCPPTIKELFVDSGLLRPMVEGQSQQFSVSENLLKEAIRAIFPSRGLARLALQFTTNPKRLQQNLFGGKFLVNVGQFNLSGALGTRGTFNFSQFFQQLGLASFLNGGRQEDLAKPLSVGLDSNSSTGTPEAAKKDGTMNKPTVGSFGFEINLQENQNALKFLASLLELPEFLLFLQHAISVPEDVARDLGDVMETVLSSQTCEQTPERLFVPSCTTEGSYEDVQCFSGECWCVNSWGKELPGSRVRGGQPRCPTDCEKQRARMQSLMGSQPAGSTLFVPACTSEGHFLPVQCFNSECYCVDAEGQAIPGTRSAIGKPKKCPTPCQLQSEQAFLRTVQALLSNSSMLPTLSDTYIPQCSTDGQWRQVQCNGPPEQVFELYQRWEAQNKGQDLTPAKLLVKIMSYREAASGNFSLFIQSLYEAGQQDVFPVLSQYPSLQDVPLAALEGKRPQPRENILLEPYLFWQILNGQLSQYPGSYSDFSTPLAHFDLRNCWCVDEAGQELEGMRSEPSKLPTCPGSCEEAKLRVLQFIRETEEIVSASNSSRFPLGESFLVAKGIRLRNEDLGLPPLFPPREAFAEQFLRGSDYAIRLAAQSTLSFYQRRRFSPDDSAGASALLRSGPYMPQCDAFGSWEPVQCHAGTGHCWCVDEKGGFIPGSLTARSLQIPQCPTTCEKSRTSGLLSSWKQARSQENPSPKDLFVPACLETGEYARLQASGAGTWCVDPASGEELRPGSSSSAQCPSLCNVLKSGVLSRRVSPGYVPACRAEDGGFSPVQCDQAQGSCWCVMDSGEEVPGTRVTGGQPACESPRCPLPFNASEVVGGTILCETISGPTGSAMQQCQLLCRQGSWSVFPPGPLICSLESGRWESQLPQPRACQRPQLWQTIQTQGHFQLQLPPGKMCSADYADLLQTFQVFILDELTARGFCQIQVKTFGTLVSIPVCNNSSVQVGCLTRERLGVNVTWKSRLEDIPVASLPDLHDIERALVGKDLLGRFTDLIQSGSFQLHLDSKTFPAETIRFLQGDHFGTSPRTWFGCSEGFYQVLTSEASQDGLGCVKCPEGSYSQDEECIPCPVGFYQEQAGSLACVPCPVGRTTISAGAFSQTHCVTDCQRNEAGLQCDQNGQYRASQKDRGSGKAFCVDGEGRRLPWWETEAPLEDSQCLMMQKFEKVPESKVIFDANAPVAVRSKVPDSEFPVMQCLTDCTEDEACSFFTVSTTEPEISCDFYAWTSDNVACMTSDQKRDALGNSKATSFGSLRCQVKVRSHGQDSPAVYLKKGQGSTTTLQKRFEPTGFQNMLSGLYNPIVFSASGANLTDAHLFCLLACDRDLCCDGFVLTQVQGGAIICGLLSSPSVLLCNVKDWMDPSEAWANATCPGVTYDQESHQVILRLGDQEFIKSLTPLEGTQDTFTNFQQVYLWKDSDMGSRPESMGCRKDTVPRPASPTEAGLTTELFSPVDLNQVIVNGNQSLSSQKHWLFKHLFSAQQANLWCLSRCVQEHSFCQLAEITESASLYFTCTLYPEAQVCDDIMESNAQGCRLILPQMPKALFRKKVILEDKVKNFYTRLPFQKLMGISIRNKVPMSEKSISNGFFECERRCDADPCCTGFGFLNVSQLKGGEVTCLTLNSLGIQMCSEENGGAWRILDCGSPDIEVHTYPFGWYQKPIAQNNAPSFCPLVVLPSLTEKVSLDSWQSLALSSVVVDPSIRHFDVAHVSTAATSNFSAVRDLCLSECSQHEACLITTLQTQPGAVRCMFYADTQSCTHSLQGQNCRLLLREEATHIYRKPGISLLSYEASVPSVPISTHGRLLGRSQAIQVGTSWKQVDQFLGVPYAAPPLAERRFQAPEPLNWTGSWDASKPRASCWQPGTRTSTSPGVSEDCLYLNVFIPQNVAPNASVLVFFHNTMDREESEGWPAIDGSFLAAVGNLIVVTASYRVGVFGFLSSGSGEVSGNWGLLDQVAALTWVQTHIRGFGGDPRRVSLAADRGGADVASIHLLTARATNSQLFRRAVLMGGSALSPAAVISHERAQQQAIALAKEVSCPMSSSQEVVSCLRQKPANVLNDAQTKLLAVSGPFHYWGPVIDGHFLREPPARALKRSLWVEVDLLIGSSQDDGLINRAKAVKQFEESRGRTSSKTAFYQALQNSLGGEDSDARVEAAATWYYSLEHSTDDYASFSRALENATRDYFIICPIIDMASAWAKRARGNVFMYHAPENYGHGSLELLADVQFALGLPFYPAYEGQFSLEEKSLSLKIMQYFSHFIRSGNPNYPYEFSRKVPTFATPWPDFVPRAGGENYKEFSELLPNRQGLKKADCSFWSKYISSLKTSADGAKGGQSAESEEEELTAGSGLREDLLSLQEPGSKTYSK

SEQ ID NO: 93 - protein, 766 aa, Homo sapiens [sequence omitted]

SEQ ID NO: 94 - protein, 431 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRGKHFISL

SEQ ID NO: 95 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD